(19) United States
(12) Patent Application Publication
(10) Pub. No.: US 2003/0104402 A1
(43) Pub. Date: Jun. 5, 2003

(54) Methods of producing or identifying intrabodies in eukaryotic cells

(75) Inventors: Maurice Zauderer, Pittsford, NY (US); Chungwen Wei, Pittsfield, NY (US); Ernest S. Smith, Ontario, NY (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: University of Rochester, Rochester, NY

(21) Appl. No.: 10/052,942

(22) Filed: Jan. 23, 2002

Related U.S. Application Data
(60) Provisional application No. 60/298,095, filed on Jun. 15, 2001.
(60) Provisional application No. 60/271,422, filed on Feb. 27, 2001.
(60) Provisional application No. 60/263,200, filed on Jan. 24, 2001.
(60) Provisional application No. 60/263,225, filed on Jan. 23, 2001.

(51) Int. Cl. C12Q 1/68; G01N 33/53; C12N 15/87

(52) U.S. Cl. 435/6; 435/7.1; 435/455

(57) ABSTRACT

The present invention relates to a high efficiency method of expressing intracellular immunoglobulin molecules in eukaryotic cells. The invention is further drawn to a method of producing intracellular immunoglobulin libraries, particularly using the trimolecular recombination method, for expression in eukaryotic cells. The invention further provides methods of selecting and screening for intracellular immunoglobulin molecules and fragments thereof. The invention also provides kits for producing, screening and selecting intracellular immunoglobulin molecules. Finally, the invention provides intracellular immunoglobulin molecules and fragments thereof, produced by the methods provided herein.

### CROSS REFERENCE TO RELATED APPLICATIONS

The present application claims benefit of the filing dates of the following applications: U.S. Provisional Application No. 60/263,225, filed Jan. 23, 2001, U.S. Provisional Application No.60/263,200, filed Jan. 24, 2001, U.S. Provisional Application No. 60/271,422, filed Feb. 27, 2001 and U.S. Provisional Application No. 60/298,095, filed Jun. 15, 2001; each of which are incorporated herein by reference in their entireties.

### BACKGROUND OF THE INVENTION

1. Field of the Invention

The present invention relates to a high efficiency method of expressing intracellular immunoglobulin molecules and fragments thereof in eukaryotic cells, a method of producing libraries of intracellular immunoglobulin molecules and libraries of intracellular immunoglobulin molecule fragments for expression in eukaryotic cells, methods of isolating intracellular immunoglobulins and fragments thereof which modify a phenotype, methods of isolating intracellular immunoglobulins and fragments thereof which bind specific antigens, and intracellular immunoglobulins and fragments thereof produced by any of these methods.

2. Related Art

The concept of intracellular immunization or intracellular inhibition has in the last decade emerged as an important strategy to counteract functionalities of pathogenic bacteria, viruses and parasites. Intracellular immunization utilizes molecular modulators such as anti-sense RNA, ribozymes, dominant negative mutants and intracellular antibodies (intrabodies) for inhibiting functional gene expression within the cell. Previous studies have shown the efficacy of intrabodies (e.g., sFvs and Fabs) targeting expression in different compartments of the cell, including the nucleus, ER, cytoplasm, golgi, plasma membrane, mitochondria, where they counteract antigens or molecules in a specific pathway. [Marasco, W. A., et al., Proc. Natl. Acad. Sci., USA 90:7889-7893 (1993); Chen, S. Y., et al., Human Gene Therapy 5:595-601 (1994); Chen, S. Y., et al., Proc Natl Acad Sci, USA 91:5932-5936 (1994); Mhashilkar, A. M., et al., Embo J 14:1542-1551 (1995); Marasco, W. A., et al., Gene Therapy 4:11-15 (1997); Richardson, J. H., et al., Proc Natl Acad Sci, USA 92:3137-3141 (1995); Duan, L., et al., Human Gene Therapy 5:1315-1324 (1994)].

Intrabodies. Expression of specific antibody molecules inside cells (intrabodies) has been shown to inhibit the function of specific proteins in a number of model systems and has important therapeutic applications (Chen, S. Y., et al., Proc. Natl. Acad. Sci. USA 91:5932-5936 (1994); Mhashilkar, A. M., et al., The EMBO J. 14:1542-1551 (1995); Richardson, J. H., et al., Proc. Natl. Acad. Sci. USA 92:3137-3141 (1995)). Use of intracellular antibodies (intrabodies) to create a phenotypic knockout of protein function might also serve as a tool for discovering the function of proteins predicted from DNA sequence data.

Usually, candidate antibodies for use as intrabodies are initially identified in the phage display screening method. In phage display methods, functional immunoglobulin domains are displayed on the surface of a phage particle which carries polynucleotide sequences encoding them. (Vaughan, T. J., et al., Nat. Biotechnol. 14:309-314 (1996); Barbas, C. F., III Nat. Med. 1:837-839 (1995); Kay, B. K., et al. (eds.) “Phage Display of Peptides and Proteins” Academic Press (1996)) In typical phage display methods, immunoglobulin fragments, e.g., Fab, Fv or disulfide stabilized Fv immunoglobulin domains are displayed as fusion proteins, i.e., fused to a phage surface protein. Examples of phage display methods that can be used to make antibodies include those disclosed in Brinkman U. et al. (1995) J. Immunol. Methods 182:41-50; Ames, R. S. et al. (1995) J. Immunol. Methods 184:177-186; Kettleborough, C. A. et al. (1994) Eur. J. Immunol. 24:952-958; Persic, L. et al. (1997) Gene 187 9-18; Burton, D. R. et al. (1994) Advances in Immunology 57:191-280; PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Phage display methods normally result in the expression of an antigen-binding fragment of an immunoglobulin molecule, thus, after phage selection, the immunoglobulin coding regions from the phage must be isolated and re-cloned to generate whole antibodies, or antigen binding fragments, and expressed in any desired host cell to test for the ability to function as an intrabody. For example, techniques to recombinantly produce Fab, Fab′ and F(ab′)2 fragments can also be employed using methods known in the art such as those disclosed in WO 92/22324; Mullinax, R. L. et al., BioTechniques 12(6):864-869 (1992); and Sawai, H. et al., AJRI 34:26-34 (1995); and Better, M. et al., Science 240: 1041-1043 (1988).

Recently, a screening method for predicting which single-chain Fv fragments (scFv) will function as intrabodies in mammalian cells was developed (Portner-Taliana et al., J. Immunological Meth. 238:161-172 (2000)). The method is a modification of the yeast two-hybrid system originally developed by Fields and Song, Nature 340:245 (1989). Some antigen-specific single-chain Fv fragments have been shown to function in the cytoplasm of yeast or mammalian cells. However, a limiting factor to the discovery of functional intrabodies is that many antibodies do not fold or function properly when they are assembled in the environment of the cell cytoplasm rather than through the normal assembly pathway of the endoplasmic reticulum (ER). The low expression levels and reduced stability associated with cytoplasmic expression is presumably due to the failure to form stabilizing disulfide bonds in the reducing environment of the cytoplasm and to reduced concentrations of ER chaperones that may be involved in protein folding. Therefore, to identify functional intrabodies extensive screening is required to identify the subset of antibodies that are able to function as intrabodies. Moreover, one large study has shown that many intrabodies that functioned when assembled in yeast did not function when assembled in the cytoplasm of mammalian cells (Visintin, M., et al., Proc. Natl. Acad. Sci. USA 96:11723-11728 (1999)). Therefore, there is a need for a method to efficiently identify or select intrabodies that function in mammalian cells.

The present inventors have developed a method that employs a unique poxvirus expression system to efficiently express a library of human-derived intracellular immunoglobulin molecules, or fragments thereof, such as scFv or Fab in the cytoplasm of higher eukaryotic cells such as mammalian cells. The method further provides a means of selecting from this library those molecules that modify a phenotype such as directly or indirectly promoting the transcriptional activation of a target gene. This method allows efficient selection of intrabodies that modify a phenotype, for example, through interaction with an unknown or unidentified gene product. The selected intrabody may then serve as a tool to characterize the specific gene product that regulates that phenotype. The method also allows the efficient selection of intrabodies specific for known proteins.

Eukaryotic Expression Libraries. A basic tool in the field of molecular biology is the conversion of poly(A)⁺ mRNA to double-stranded (ds) cDNA, which then can be inserted into a cloning vector and expressed in an appropriate host cell. A method common to many cDNA cloning strategies involves the construction of a “cDNA library” which is a collection of cDNA clones derived from the poly(A)⁺ MRNA derived from a cell of the organism of interest. For example, in order to isolate cDNAs which express immunoglobulin genes, a cDNA library might be prepared from pre B cells, B cells, or plasma cells. Methods of constructing cDNA libraries in different expression vectors, including filamentous bacteriophage, bacteriophage lambda, cosmids, and plasmid vectors, are known. Some commonly used methods are described, for example, in Sambrook et al., Molecular Cloning: A Laboratory Manual, 2d Edition, Cold Spring Harbor Laboratory, publisher, Cold Spring Harbor, N.Y. (1990).

Many different methods of isolating target genes from CDNA libraries have been utilized, with varying success. These include, for example, the use of nucleic acid hybridization probes, which are labeled nucleic acid fragments having sequences complementary to the DNA sequence of the target gene. When this method is applied to cDNA clones in transformed bacterial hosts, colonies or plaques hybridizing strongly to the probe are likely to contain the target DNA sequences. Hybridization methods, however, do not require, and do not measure, whether a particular cDNA clone is expressed. Alternative screening methods rely on expression in the bacterial host, for example, colonies or plaques can be screened by immunoassay for binding to antibodies raised against the protein of interest. Assays for expression in bacterial hosts are often impeded, however, because the protein may not be sufficiently expressed in bacterial hosts, it may be expressed in the wrong conformation, and it may not be processed, and/or transported as it would in a eukaryotic system. Many of these problems have been encountered in attempts to produce immunoglobulin molecules in bacterial hosts, as alluded to above.

Accordingly, use of mammalian expression libraries to isolate cDNAs encoding immunoglobulin molecules would offer several advantages over bacterial libraries. For example, immunoglobulin molecules, and subunits thereof, expressed in eukaryotic hosts should be functional and should undergo any normal posttranslational modification. A protein ordinarily transported through the intracellular membrane system to the cell surface should undergo the complete transport process. Further, use of a eukaryotic system would make it possible to isolate polynucleotides based on functional expression of eukaryotic RNA or protein. For example, immunoglobulin molecules could be isolated based on their specificity for a given antigen.

With the exception of some recent lymphokine cDNAs isolated by expression in COS cells (Wong, G. G., et al., Science 228:810-815 (1985); Lee, F. et al., Proc. Natl. Acad. Sci. USA 83:2061-2065 (1986); Yokota, T., et al., Proc. Natl. Acad. Sci. USA 83:5894-5898 (1986); Yang, Y., et al., Cell 47:3-10 (1986)), few cDNAs have been isolated from mammalian expression libraries. There appear to be two principal reasons for this: First, the existing technology (Okayama, H. et al., Mol. Cell. Biol. 2:161-170 (1982)) for construction of large plasmid libraries is difficult to master, and library size rarely approaches that accessible by phage cloning techniques. (Huynh, T. et al., In: DNA Cloning Vol, I, A Practical Approach, Glover, D. M. (ed.), IRL Press, Oxford (1985), pp. 49-78). Second, the existing vectors are, with one exception (Wong, G. G., et al., Science 228:810-815 (1985)), poorly adapted for high level expression. Thus, expression in mammalian hosts previously has been most frequently employed solely as a means of verifying the identity of the protein encoded by a gene isolated by more traditional cloning methods.

Poxvirus Vectors. Poxvirus vectors are used extensively as expression vehicles for protein and antigen expression in eukaryotic cells. The ease of cloning and propagating vaccinia in a variety of host cells has led to the widespread use of poxvirus vectors for expression of foreign protein and as vaccine delivery vehicles (Moss, B., Science 252:1662-7 (1991)).

Large DNA viruses are particularly useful expression vectors for the study of cellular processes as they can express many different proteins in their native form in a variety of cell lines. In addition, gene products expressed in recombinant vaccinia virus have been shown to be efficiently processed and presented in association with MHC class I for stimulation of cytotoxic T cells. The gene of interest is normally cloned in a plasmid under the control of a promoter flanked by sequences homologous to a non-essential region in the virus and the cassette is introduced into the genome via homologous recombination. A panoply of vectors for expression, selection and detection have been devised to accommodate a variety of cloning and expression strategies. However, homologous recombination is an ineffective means of making a recombinant virus in situations requiring the generation of complex libraries or when the insert DNA is large. An alternative strategy for the construction of recombinant genomes relying on direct ligation of viral DNA “arms” to an insert and the subsequent rescue of infectious virus has been explored for the genomes of poxvirus (Merchlinsky, et al., 1992, Virology 190:522-526; Pfleiderer, et al., 1995, J. General Virology 76:2957-2962; Scheiflinger, et al., 1992, Proc. Natl. Acad. Sci. USA 89:9977-9981), herpesvirus (Rixon, et al., 1990, J. General Virology 71:2931-2939) and baculovirus (Ernst, et al., 1994, Nucleic Acids Research 22:2855-2856).

Poxviruses are ubiquitous vectors for studies in eukaryotic cells as they are easily constructed and engineered to express foreign proteins at high levels. The wide host range of the virus allows one to faithfully express proteins in a variety of cell types. Direct cloning strategies have been devised to extend the scope of applications for poxvirus viral chimeras in which the recombinant genomes are constructed in vitro by direct ligation of DNA fragments to vaccinia “arms” and transfection of the DNA mixture into cells infected with a helper virus (Merchlinsky, et al., 1992, Virology 190:522-526; Scheiflinger, et al., 1992, Proc. Natl. Acad. Sci. USA 89:9977-9981). This approach has been used for high level expression of foreign proteins (Pfleiderer, et al., 1995, J. Gen. Virology 76:2957-2962) and to efficiently clone fragments as large as 26 kilobases in length (Merchlinsky, et al., 1992, Virology 190:522-526).

Naked vaccinia virus DNA is not infectious because the virus cannot utilize cellular transcriptional machinery and relies on its own proteins for the synthesis of viral RNA. Previously, temperature sensitive conditional lethal (Merchlinsky, et al., 1992, Virology 190:522-526) or non-homologous poxvirus fowlpox (Scheiflinger, et al., 1992, Proc. Natl. Acad. Sci. USA 89:9977-9981) have been utilized as helper virus for packaging. An ideal helper virus will efficiently generate infectious virus but not replicate in the host cell or recombine with the vaccinia DNA products. Fowlpox virus has the properties of an ideal helper virus as it is used at 37° C., will not revert to a highly replicating strain, and, since it does not recombine with vaccinia DNA or productively infect primate cell lines, can be used at relatively high multiplicity of infection (MOI).

The utility of the vaccinia based direct ligation vector vNotI/tk, has been described by Merchlinsky, et al. (1992, Virology 190:522-526). This genome lacks the NotI site normally present in the HindIII F fragment and contains a unique NotI site at the beginning of the thymidine kinase gene in frame with the coding sequence. This allows the insertion of DNA fragments into the NotI site and the identification of recombinant genomes by drug selection. The vNotI/tk vector will only express foreign proteins at the level of the thymidine kinase gene, a weakly expressed gene only made early during viral infection. Thus, the vNotI/tk vector can be used to efficiently clone large DNA fragments but does not fix the orientation of the DNA insert or lead to high expression of the foreign protein.

Customarily, a foreign protein coding sequence is introduced into the poxvirus genome by homologous recombination with infectious virus. In this traditional method, a previously isolated foreign DNA is cloned in a transfer plasmid behind a vaccinia promoter flanked by sequences homologous to a region in the poxvirus which is non-essential for viral replication. The transfer plasmid is introduced into poxvirus-infected cells to allow the transfer plasmid and poxvirus genome to recombine in vivo via homologous recombination. As a result of the homologous recombination, the foreign DNA is transferred to the viral genome.

Although traditional homologous recombination in poxviruses is useful for expression of previously isolated foreign DNA in a poxvirus, the method is not conducive to the construction of libraries, since the overwhelming majority of viruses recovered have not acquired a foreign DNA insert. Using traditional homologous recombination, the recombination efficiency is in the range of approximately 0.1% or less. Thus, the use of poxvirus vectors has been limited to subcloning of previously isolated DNA molecules for the purposes of protein expression and vaccine development.

Alternative methods using direct ligation vectors have been developed to efficiently construct chimeric genomes in situations not readily amenable for homologous recombination (Merchlinsky, M. et al., 1992, Virology 190:522-526; Scheiflinger, F. et al., 1992, Proc. Natl. Acad. Sci. USA. 89:9977-9981). In such protocols, the DNA from the genome is digested, ligated to insert DNA in vitro, and transfected into cells infected with a helper virus (Merchlinsky, M. et al., 1992, Virology 190:522-526, Scheiflinger, F. et al., 1992, Proc. Natl. Acad. Sci.

USA 89:9977-9981). In one protocol, the genome was digested at a unique NotI site and a DNA insert containing elements for selection or detection of the chimeric genome was ligated to the genomic arms (Scheiflinger, F. et al., 1992, Proc. Natl. Acad. Sci. USA. 89:9977-9981). This direct ligation method was described for the insertion of foreign DNA into the vaccinia virus genome (Pfleiderer et al., 1995, J. General Virology 76:2957-2962). Alternatively, the vaccinia WR genome was modified by removing the NotI site in the HindIII F fragment and reintroducing a NotI site proximal to the thymidine kinase gene such that insertion of a sequence at this locus disrupts the thymidine kinase gene, allowing isolation of chimeric genomes via use of drug selection (Merchlinsky, M. et al., 1992, Virology 190:522-526).

The direct ligation vector vNotI/tk allows one to efficiently clone and propagate previously isolated DNA inserts at least 26 kilobase pairs in length (Merchlinsky, M. et al., 1992, Virology, 190:522-526). Although large DNA fragments are efficiently cloned into the genome, proteins encoded by the DNA insert will only be expressed at the low level corresponding to the thymidine kinase gene, a relatively weakly expressed early class gene in vaccinia. In addition, the DNA will be inserted in both orientations at the NotI site, and therefore might not be expressed at all. Additionally, although the recombination efficiency using direct ligation is higher than that observed with traditional homologous recombination, the resulting titer is relatively low.

Accordingly, poxvirus vectors were previously not used to identify previously unknown genes of interest from a complex population of clones, because a high efficiency, high titer-producing method of cloning did not exist for poxviruses. More recently, however, the present inventor developed a method for generating recombinant poxviruses using tri-molecular recombination. See Zauderer, WO 00/028016, published May 18, 2000, and Zauderer, WO 01/72995, published Oct. 4, 2001, both of which are incorporated herein by reference in their entireties.

Tri-molecular recombination is a novel, high efficiency, high titer-producing method for producing recombinant poxviruses. Using the tri-molecular recombination method in vaccinia virus, the present inventor has achieved recombination efficiencies of at least 90%, and titers at least 2 orders of magnitude higher, than those obtained by direct ligation. According to the tri-molecular recombination method, a poxvirus genome is cleaved to produce two nonhomologous fragments or “arms.” A transfer vectoris produced which carries the heterologous insert DNA flanked by regions of homology with the two poxvirus arms. The arms and the transfer vector are delivered into a recipient host cell, allowing the three DNA molecules to recombine in vivo. As a result of the recombination, a single poxvirus genome molecule is produced which comprises each of the two poxvirus arms and the insert DNA.

### SUMMARY OF THE INVENTION

In accordance with one aspect of the present invention, there is provided a method of selecting or identifying polynucleotides which encode an intracellular immunoglobulin molecule, or fragment thereof, from libraries of polynucleotides expressed in eukaryotic cells.

Also provided is a method of constructing libraries of polynucleotides encoding intracellular immunoglobulin molecules, or fragments thereof in eukaryotic cells using virus vectors, where the libraries are constructed by trimolecular recombination.

Further provided are methods of identifying host cells expressing intracellular immunoglobulin molecules, or fragments thereof, by selecting and/or screening for a modified phenotype.

### BRIEF DESCRIPTION OF THE FIGURES

FIG. 1. Construction of pVHEc.

FIG. 2. Construction of pVKEc, pVLEc, pVKEn, and pVLEn.

FIG. 3. Construction of pVP16AD-VHEn.

FIG. 4. Construction of pGAL4BD-Ag.

FIG. 5. Construction of pG5-R.

FIG. 6. Schematic of the Tri-Molecular Recombination Method.

FIG. 7. Nucleotide Sequence of p7.5/tk and pEL/tk. The nucleotide sequence of the promoter and beginning of the thymidine kinase gene for v7.5/tk and vEL/tk is shown.

FIG. 8. Modifications in the nucleotide sequence of the p7.5/tk (SEQ ID NO:150) vaccinia transfer plasmid. Four new vectors, p7.5/ATGO/tk (SEQ ID NO:151), p7.5/ATG1/tk (SEQ ID NO:152), p7.5/ATG2/tk (SEQ ID NO:153) and p7.5/ATG3/tk (SEQ ID NO:9) have been derived as described in the text from the p7.5/tk vaccinia transfer plasmid.

FIG. 9. Attenuation of poxvirus-mediated cytopathic effects.

FIG. 10. Construction of scFv expression vectors.

### DETAILED DESCRIPTION OF THE PREFERRED EMBODIMENTS

The present invention is broadly directed to methods of identifying and/or producing intracellular immunoglobulin molecules (Ig) or fragments thereof in a eukaryotic system. In addition, the invention is directed to methods of identifying polynucleotides which encode an intracellular Ig or Ig fragment from complex expression libraries of polynucleotides encoding such intracellular immunoglobulin molecules or fragments, where the libraries are constructed and screened in eukaryotic host cells. Further embodiments include an isolated intracellular immunoglobulin molecule or fragment thereof, produced by any of the above methods, and a kit allowing production of such isolated intracellular immunoglobulins.

A particularly preferred aspect of the present invention is the construction of complex intracellular immunoglobulin libraries in eukaryotic host cells using poxvirus vectors constructed by trimolecular recombination. The ability to construct complex cDNA libraries in a pox virus based vector and to select and/or screen for specific recombinants on the basis of a modified phenotype can be the basis for identification of intracellular immunoglobulins, particularly human intracellular immunoglobulins, in eukaryotic cells. It would overcome the limitations of synthesis and assembly in bacteria or yeast.

It is to be noted that the term “phenotype” refers to the total physical and biochemical characteristics displayed by host cells under a particular set of environmental factors, regardless of the actual genotype of the organism. The term “modified phenotype” refers to a change in the form, character, or intensity of a physical or biochemical characteristic displayed by host cells under a particular set of environmental factors. A phenotype might be displayed by a given host cell in response to any number of environmental factors including, but not limited to temperature, exposure to certain molecules, or signalling by another cell. In certain embodiments a given predetermined phenotype, and any modifications of that phenotype may be those which occur naturally in a given host cell. In alternative embodiments, a host cell is engineered such that a more easily detectable phenotype is substituted into a transcriptional pathway of interest, for example, a reporter gene may be inserted in operable association with a promoter in a cellular regulatory pathway of interest. In either case, it is preferred that the phenotype of interest, and any modifications of that phenotype that are contemplated, are “predetermined,” i.e., they are known and well characterized, and are readily detectable in the host cell used to screen and/or select for intracellular immunoglobulins, or fragments thereof of the present invention.

Furthermore, an intracellular immunoglobulin molecule, or fragment thereof of the present invention is selected and/or screened for by its ability to “induce” a in a given host cell. In this context, the term “induce” is used herein to describe the ability of the intracellular immunoglobulin, or fragment thereof, to effect, either directly or indirectly, a change in the form, character, or intensity of a physical or biochemical characteristic displayed by the given host cells under a particular set of environmental factors. Thus, the action of the intracellular immunoglobulin, or fragment thereof, on the given phenotype may be direct, for example, activating or suppressing transcription of the gene product actually responsible for the modified phenotype, or indirect, for example, activating or suppressing expression of a gene in a signal transduction pathway which is far removed from the actual gene product responsible for the modified phenotype.

It is to be noted that the term “a” or “an” entity, refers to one or more of that entity; for example, “an intracellular immunoglobulin molecule,” is understood to represent one or more intracellular immunoglobulin molecules. As such, the terms “a” (or “an”), “one or more,” and “at least one” can be used interchangeably herein.

The term “eukaryote” or “eukaryotic organism” is intended to encompass all organisms in the animal, plant, and protist kingdoms, including protozoa, fungi, yeasts, green algae, single celled plants, multi celled plants, and all animals, both vertebrates and invertebrates. The term does not encompass bacteria or viruses. A “eukaryotic cell” is intended to encompass a singular “eukaryotic cell” as well as plural “eukaryotic cells,” and comprises cells derived from a eukaryote.

The term “vertebrate” is intended to encompass a singular “vertebrate” as well as plural “vertebrates,” and comprises mammals and birds, as well as fish, reptiles, and amphibians.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. Preferably, the mammal is a human subject.

The terms “tissue culture” or “cell culture” or “culture” or “culturing” refer to the maintenance or growth of plant or animal tissue or cells in vitro under conditions that allow preservation of cell architecture, preservation of cell function, further differentiation, or all three. “Primary tissue cells” are those taken directly from tissue, i.e., a population of cells of the same kind performing the same function in an organism. Treating such tissue cells with the proteolytic enzyme trypsin, for example, dissociates them into individual primary tissue cells that grow or maintain cell architecture when seeded onto culture plates. Cell cultures arising from multiplication of primary cells in tissue culture are called “secondary cell cultures.” Most secondary cells divide a finite number of times and then die. A few secondary cells, however, may pass through this “crisis period,” after which they are able to multiply indefinitely to form a continuous “cell line.” The liquid medium in which cells are cultured is referred to herein as “culture medium” or “culture media.”

The term “polynucleotide” refers to any one or more nucleic acid segments, or nucleic acid molecules, e.g., DNA or RNA fragments, present in a nucleic acid or construct. A “polynucleotide encoding an intracellular immunoglobulin subunit polypeptide or intracellular immunoglobulin fragment” refers to a polynucleotide which comprises the coding region for such a polypeptide. In addition, a polynucleotide may encode a regulatory element such as a promoter or a transcription terminator, or may encode a specific element of a polypeptide or protein, such as a secretory signal peptide or a functional domain. As used herein, the term “identify” refers to methods in which desired molecules, e.g., polynucleotides encoding intracellular immunoglobulin molecules, or fragments thereof, are distinguished from a plurality or library of such molecules. Identification methods include “selection” and “screening.” As used herein, “selection” methods are those in which the desired molecules may be directly separated from the library. For example, in one selection method described herein, host cells comprising the desired polynucleotides are directly separated from the host cells comprising the remainder of the library by becoming nonadherent, e.g., undergoing a lytic event, and thereby being released from the substrate to which the remainder of the host cells are attached. For another example, FACS (fluorescence-activated cells sorting) is used to separate cells exhibiting the modified phenotype from the remainder of the host cells which do not exhibit the modified phenotype. As used herein, “screening” methods are those in which pools comprising the host cells are subjected to an assay in which the modified phenotype can be detected. For example, aliquots of the pools containing host cells which exhibit the modified phenotype may then divided into successively smaller pools which are likewise assayed, until a pool which is highly enriched for those host cells is achieved.

Immunoglobulins. As used herein, an “immunoglobulin” or “immunoglobulin molecule” is a complete, bi-molecular immunoglobulin, e.g., generally comprising four “subunit polypeptides,” i.e., two identical heavy chains and two identical light chains. In some instances, e.g., immunoglobulin molecules derived from camelid species or engineered based on camelid immunglobulins, a complete immunoglobulin molecule may consist of heavy chains only, with no light chains. See, e.g., Hamers-Casterman et al., Nature 363:446-448 (1993). Thus, by a “subunit polypeptide,” when referring to an immunoglobulin, is meant a single heavy chain polypeptide or a single light chain polypeptide comprising V and C domains. Immunoglobulin molecules are also referred to as “antibodies” or “Igs” and the terms are used interchangeably herein. An “isolated immunoglobulin” refers to an immunoglobulin molecule, or two or more immunoglobulin molecules, which are substantially removed from the milieu of proteins and other substances, and which bind a specific antigen.

As used herein, an “immunoglobulin fragment” is a portion of an immunoglobulin which includes an antigen-binding domain, e.g., VH or VL. Intracellular immunoglobulin fragments of the present invention preferably lack a signal peptide, membrane spanning domain, and/or intracellular domains necessary for secretion or expression on the cell surface. Immunoglobulin fragments also include smaller fragments such as Fv, Fab, Fab′, F(ab′)₂, disulfide-linked Fvs (sdFv), and Fab minibodies. As is known in the art, Fv comprises a VH domain and a VL domain, Fab comprises VH joined to CH1 and an L chain, a Fab minibody comprises a fusion of CH3 domain to Fab.

Immunoglobulin fragments also include “single-chain fragments,” such as single-chain Fv (scFv or sFv), diabodies, triabodies, tetrabodies, scFv minibodies, and dimeric scFv. As is known in the art, scFv comprises VH joined to VL by a peptide linker, usually 15-20 residues in length, diabodies comprise scFv with a peptide linker about 5 residues in length, triabodies comprise scFv with no peptide linker, tetrabodies comprise scFv with peptide linker 1 residue in length, a scFv minibody comprises a fusion of CH3 domain to scFv, and dimeric scFv comprise a fusion of two scFvs in tandem using another peptide linker (reviewed in Chames and Baty, FEMS Microbiol. Letts. 189:1-8 (2000)). Preferably, an immunoglobulin fragment includes both antigen binding domains, i.e., VH and VL. However, in certain embodiments, immunoglobulin fragments may also comprise a V_{H}H domain derived from a camelid antibody. The V_{H}H may be engineered to include CDRs from other species, for example, from human antibodies. Alternatively, a human-derived heavy chain V_{H} fragment may be engineered to resemble a single-chain camelid CDR, a process referred to as “camelization.” See, e.g., Davies J., and Riechmann, L., FEBS Letters 339:285-290 (1994), and Riechmann, L., and Muyldermans, S., J. Immunol. Meth. 231:25-38 (1999), both of which are incorporated herein by reference in their entireties. Other immunoglobulin fragments are well known in the art and disclosed in well-known reference materials such as those described herein. Immunoglobulin fragments are also referred to as “antibody fragments” or “Ig fragments” and the terms are used interchangeably herein. An “isolated immunoglobulin fragment” refers to an immunoglobulin fragment, or two or more immunoglobulin fragments, which are substantially removed from the milieu of proteins and other substances, and which include an antigen-binding domain.

The heavy chain, which determines the “class” of the immunoglobulin molecule, is the larger of the two subunit polypeptides, and in nature comprises a variable region and a constant region. By “heavy chain” is meant a full-length secreted heavy chain form, i.e., one that is released from the cell, a membrane bound heavy chain form, i.e., comprising a membrane spanning domain and an intracellular domain, or a fragment thereof lacking a membrane spanning domain and an intracellular domain. The membrane spanning and intracellular domains can be the naturally-occurring domains associated with a certain heavy chain, i.e., the domain found on memory B-cells, or it may be a heterologous membrane spanning and intracellular domain, e.g., from a different immunoglobulin class or from a heterologous polypeptide, i.e., a non-immunoglobulin polypeptide. As will become apparent, the present invention is preferably carried out using immunoglobulin fragments lacking the membrane spanning and intracellular domains. Immunoglobulin “classes” refer to the broad groups of immunoglobulins which serve different functions in the host. For example, human immunoglobulins are divided into five classes, i.e., IgG, comprising a γ heavy chain, IgM, comprising a μ heavy chain, IgA, comprising an a heavy chain, IgE, comprising an ε heavy chain, and IgD, comprising a δ heavy chain. Certain classes of immunoglobulins are also further divided into “subclasses.” For example, in humans, there are four different IgG subclasses, IgGI, IgG2, IgG3, and IgG4 comprising γ-1, γ-2, γ-3, and γ-4 heavy chains, respectively, and two different IgA subclasses, IgA-1 and IgA-2, comprising α-1 and α-2 heavy chains, respectively. It is to be noted that the class and subclass designations of immunoglobulins vary between animal species, and certain animal species may comprise additional classes of immunoglobulins. For example, birds also produce IgY, which is found in egg yolk.

By “light chain” is meant the smaller immunoglobulin subunit which associates with the amino terminal region of a heavy chain. In complete immunoglobulins, as with a heavy chain, a light chain comprises a variable region and a constant region. There are two different kinds of light chains, κ and λ, and a pair of these can associate with a pair of any of the various heavy chains to form an immunoglobulin molecule.

In complete immunoglobulins, immunoglobulin subunit polypeptides each comprise a constant region and a variable region. The heavy chain variable region, or V_{H} domain, and the light chain variable region, e.g., a V_{K} or a V_{L} domain, combine to form a “complementarity determining region” or CDR, the portion of an immunoglobulin molecule which specifically recognizes an antigenic epitope. In camelid species, however, the heavy chain variable region, referred to as V_{H}H, forms the entire CDR. The main differences between camelid V_{H}H variable regions and those derived from conventional antibodies (V_{H}) include (a) more hydrophobic amino acids in the light chain contact surface of V_{H} as compared to the corresponding region in V_{H}H, (b) a longer CDR3 in V_{H}H, and (c) the frequent occurrence of a disulfide bond between CDR1 and CDR3 in V_{H}H. Each complete immunoglobulin molecule comprises two identical CDRs. A large repertoire of variable regions associated with heavy and light chain constant regions are produced upon differentiation of antibody-producing cells in an animal through rearrangements of a series of germ line DNA segments which results in the formation of a gene which encodes a given variable region. Further variations of heavy and light chain variable regions take place through somatic mutations in differentiated cells. The structure and in vivo formation of immunoglobulin molecules is well understood by those of ordinary skill in the art of immunology. Concise reviews of the generation of immunoglobulin diversity may be found, e.g., in Harlow and Lane, Antibodies, A Laboratory Manual Cold Spring Harbor Laboratory, Cold Spring Harbor, N.Y. (1988) (hereinafter, “Harlow”); and Roitt, et al., Immunology Gower Medical Publishing, Ltd., London (1985) (hereinafter, “Roitt”). Harlow and Roitt are incorporated herein by reference in their entireties.

Intracellular immunoglobulin molecules, and fragments thereof, of the present invention may be from any animal origin including birds, fish, and mammals. Preferably, the antibodies and fragments are of human, mouse, dog, cat, rabbit, goat, guinea pig, camel, llama, horse, or chicken origin. Most preferably, the antibodies and fragments are of human origin. In a preferred aspect of the present invention, intracellular immunoglobulins are identified which specifically interact with intracellular antigens, e.g., human intracellular immunoglobulins which specifically bind human intracellular antigens.

As used herein, an “intracellular immunoglobulin molecule” is a complete immunoglobulin which is the same as a naturally-occurring secreted immunoglobulin, but which remains inside of the cell following synthesis. An “intracellular immunoglobulin fragment” refers to any fragment, including single-chain fragments of an intracellular immunoglobulin molecule. Thus, an intracellular immunoglobulin molecule or fragment thereof is not secreted or expressed on the outer surface of the cell. Single-chain intracellular immunoglobulin fragments are referred to herein as “single-chain immunoglobulins.” As used herein, the term “intracellular immunoglobulin molecule or fragment thereof” is understood to encompass an “intracellular immunoglobulin,” a “single-chain intracellular immunoglobulin” (or fragment thereof), an “intracellular immunoglobulin fragment,” an “intracellular antibody” (or fragment thereof), and an “intrabody” (or fragment thereof). As such, the terms “intracellular immunoglobulin,” “intracellular Ig,” “intracellular antibody,” and “intrabody” may be used interchangeably herein, and are all encompassed by the generic definition of an “intracellular immunoglobulin molecule, or fragment thereof.” An intracellular immunoglobulin molecule, or fragment thereof of the present invention may, in some embodiments, comprise two or more subunit polypeptides, e.g., a “first intracellular immunoglobulin subunit polypeptide” and a “second intracellular immunoglobulin subunit polypeptide.” However, in other embodiments, an intracellular immunoglobulin may be a “single-chain intracellular immunoglobulin,” i.e., including only a single polypeptide. As used herein, a “single-chain intracellular immunoglobulin” is defined as any unitary fragment that has a desired activity, for example, intracellular binding to an antigen. Thus, single-chain intracellular immunoglobulins encompass those which comprise both heavy and light chain variable regions which act together to bind antigen, as well as single-chain intracellular immunoglobulins which only have a single variable region which binds antigen, for example, a “camelized” heavy chain variable region as described herein. An intracellular immunoglobulin or Ig fragment may be expressed anywhere substantially within the cell, such as in the cytoplasm, on the inner surface of the cell membrane, or in a subcellular compartment (also referred to as cell subcompartment or cell compartment) such as the nucleus, golgi, endoplasmic reticulum, endosome, mitochondria, etc. Additional cell subcompartments include those that are described herein and well known in the art.

In certain embodiments, the present invention is drawn to methods to identify, i.e., select or alternatively screen for, polynucleotides which singly (e.g., single-chain fragments) or collectively encode intracellular immunoglobulin molecules, or fragments thereof. In related embodiments, the present invention is drawn to isolated intracellular immunoglobulin molecules and fragments thereof encoded by the polynucleotides identified by these methods.

Where the intracellular immunoglobulin molecules, or fragments thereof, are composed of two subunit polypeptides (and therefore encoded by two polynucleotides), preferred methods comprise a two-step screening and/or selection process. In the first step, a polynucleotide encoding a first intracellular immunoglobulin subunit, i.e., either a heavy chain or a light chain, is identified from a library of polynucleotides encoding that subunit by introducing the library into a population of eukaryotic host cells, and expressing the intracellular immunoglobulin subunit in combination with one or more species of a second intracellular immunoglobulin subunit, where the second intracellular immunoglobulin subunit is not the same as the first intracellular immunoglobulin subunit, i.e., if the first intracellular immunoglobulin subunit polypeptide is a heavy chain polypeptide, the second intracellular immunoglobulin subunit polypeptide will be a light chain polypeptide.

Once one or more polynucleotides encoding one or more first intracellular immunoglobulin subunits are isolated from the library in the first step, and a second intracellular immunoglobulin subunit is identified in the second step. Isolated polynucleotides encoding the isolated first intracellular immunoglobulin subunit polypeptide(s) are transferred into and expressed in host cells in which a library of polynucleotides encoding the second intracellular immunoglobulin subunit are expressed, thereby allowing identification of a polynucleotide encoding a second intracellular immunoglobulin subunit polypeptide which, when combined with the first intracellular immunoglobulin subunit identified in the first step, forms a functional intracellular immunoglobulin molecule, or fragment thereof, which modifies a predetermined phenotype or which binds a particular antigen. In certain embodiments, intracellular immunoglobulin molecules, or fragments thereof are identified through screening and/or selecting for host cells which exhibit a modified phenotype. Thus, the methods comprise a number of different ways to select and/or screen for cells containing intracellular immunoglobulin molecules, or fragments thereof, as described below.

Where intracellular immunoglobulin fragments are composed of one polypeptide (i.e., a single-chain fragment) (therefore encoded by one polynucleotide), preferred methods comprise a one-step screening and/or selection process. Polynucleotides encoding a single-chain fragment, comprising a heavy chain variable region and a light chain variable region, or alternatively, a camelized heavy chain variable region, are identified from a library by introducing the library into host cells such as eukaryotic cells and recovering polynucleotides of said library from those host cells which exhibit a desired, predetermined modified phenotype.

As used herein, a “library” is a representative genus of polynucleotides, i.e., a group of polynucleotides related through, for example, their origin from a single animal species, tissue type, organ, or cell type, where the library collectively comprises at least two different species within a given genus of polynucleotides. A library of polynucleotides preferably comprises at least 10, 100, 10³, 10⁴, 10⁵, 10⁶, 10⁷, 10⁸, or 10⁹ different species within a given genus of polynucleotides. More specifically, in certain embodiments, a library of the present invention encodes a plurality of a certain intracellular immunoglobulin subunit polypeptides, i.e., either a heavy chain subunit polypeptide or a light chain subunit polypeptide. The heavy chain subunit polypeptide or light chain subunit polypeptide preferably comprises a variable region. In this context, a “library” of the present invention optionally comprises polynucleotides encoding an polypeptide of a certain type and class e.g., a library might encode a human μ, γ-1, γ-2, γ-3, γ-4, α-1, α-2, ε, or δ heavy chain, or a human κ or λ light chain, or a domain thereof. In other embodiments, the library encodes a plurality of intracellular immunoglobulin single-chain fragments which comprise a variable region, such as a light chain variable region or a heavy chain variable region, and preferably comprises both a light chain variable region and a heavy chain variable region. Optionally, such a library comprises polynucleotides encoding an intracellular immunoglobulin subunit polypeptide of a certain type and class, or domains thereof.

Although each member of any one library of the present invention optionally encodes the same heavy or light chain constant region, the library will collectively comprise at least two, preferably at least 10, 100, 10³, 10⁴, 10⁵, 10⁶, 10⁷, 10⁸, or 10⁹ different variable regions i.e., a “plurality” of variable regions optionally associated with the common constant region.

In one aspect, the present invention encompasses methods to produce libraries of polynucleotides encoding intracellular immunoglobulin subunit polypeptides or intracellular immunoglobulin fragments. Furthermore, the present invention encompasses libraries of intracellular immunoglobulin subunit polypeptidces or intracellular immunoglobulin fragments constructed in eukaryotic expression vectors according to the methods described herein. Such libraries are preferably produced in eukaryotic virus vectors, even more preferably in poxvirus vectors. Such methods and libraries are described herein.

By “recipient cell” or “host cell” or “cell” is meant a cell or population of cells into which polynucleotide libraries of the present invention are introduced. A host cell of the present invention is preferably a eukaryotic cell or cell line, preferably a plant, animal, vertebrate, mammalian, rodent, mouse, primate, or human cell or cell line. By “a population of host cells” is meant a group of cultured cells into which a “library” of the present invention can be introduced and expressed. Any host cells which will support expression from a given library constructed in a given vector is intended. Suitable and preferred host cells are disclosed herein. Furthermore, certain host cells which are preferred for use with specific vectors and with specific selection and/or screening schemes are disclosed herein. Although it is preferred that a population of host cells be a monoculture, i.e., where each cell in the population is of the same cell type, mixed cultures of cells are also contemplated. Host cells of the present invention may be adherent, i.e., host cells which grow attached to a solid substrate, or, alternatively, the host cells may be in suspension. Host cells may be cells derived from primary tumors, cells derived from metastatic tumors, primary cells, cells which have lost contact inhibition, transformed primary cells, immortalized primary cells, cells which may undergo apoptosis, and cell lines derived therefrom. Additionally, the methods of the invention may exclude certain cells as host cells. For example, the methods may exclude yeast or other lower eukaryotes, or may exclude nonvertebrates.

As noted above, one method to identify intracellular immunoglobulin molecules or fragments comprises the introduction of a “first” library of polynucleotides into a population of host cells, as well as a “second” library of polynucleotides into the same population of host cells. The first and second libraries are complementary, i.e., if the “first” library encodes intracellular immunoglobulin heavy chains, the “second” library will encode intracellular immunoglobulin light chains, thereby allowing assembly of intracellular immunoglobulin molecules, or fragments thereof, in the population of host cells. Also, as noted above, another method to identify intracellular immunoglobulin molecules, or fragments thereof, comprises introduction of a single library of polynucleotides encoding single-chain fragments into a population of host cells. The description of polynucleotide libraries, the composition of the polynucleotides in the library, and the polypeptides encoded by the polynucleotides therefore encompass the polynucleotides which comprise each of these libraries, and the polypeptides encoded thereby. The libraries may be constructed in any suitable vectors. The first and second libraries may, but need not be, constructed in the same vector. Suitable and preferred vectors for the these libraries are disclosed herein.

Polynucleotides contained in libraries of the present invention encode intracellular immunoglobulin subunit polypeptides or immunoglobulin fragments through “operable association with a transcriptional control region.” One or more nucleic acid molecules in a given polynucleotide are “operably associated” when they are placed into a functional relationship. This relationship can be between a coding region for a polypeptide and a regulatory sequence(s) which are connected in such a way as to permit expression of the coding region when the appropriate molecules (e.g., transcriptional activator proteins, polymerases, etc.) are bound to the regulatory sequences(s). “Transcriptional control regions” include, but are not limited to promoters, enhancers, operators, and transcription termination signals, and are included with the polynucleotide to direct its transcription. For example, a promoter would be operably associated with a nucleic acid molecule if the promoter was capable of effecting transcription of that nucleic acid molecule. Generally, “operably associated” means that the DNA sequences are contiguous or closely connected in a polynucleotide. However, some transcription control regions, e.g., enhancers, do not have to be contiguous.

By “control sequences” or “control regions” is meant DNA sequences necessary for the expression of an operably associated coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhances.

A variety of transcriptional control regions are known to those skilled in the art. Preferred transcriptional control regions include those which function in vertebrate cells, such as, but not limited to, promoter and enhancer sequences from poxviruses, adenoviruses, herpesviruses, e.g., human cytomegalovirus (preferably the intermediate early promoter, preferably in conjunction with intron-A), simian virus 40 (preferably the early promoter), retroviruses (such as Rous sarcoma virus), and picornaviruses (particularly an internal ribosome entry site, or IRES, enhancer region, also referred to herein as a CITE sequence). Other preferred transcriptional control regions include those derived from mammalian genes such as actin, heat shock protein, and bovine growth hormone, as well as other sequences capable of controlling gene expression in eukaryotic cells. Additional suitable transcription control regions include tissue-specific promoters and enhancers as well as inducible promoters (e.g., promoters inducible by tetracycline, and temperature sensitive promoters). As will be discussed in more detail below, especially preferred are promoters capable of functioning in the cytoplasm of poxvirus-infected cells.

In certain preferred embodiments in the context of an intracellular immunoglobulin fragment, each subunit polypeptide, e.g., either a “first intracellular immunoglobulin subunit polypeptide” or a “second intracellular immunoglobulin subunit polypeptide” comprises an immunoglobulin variable region selected from the group consisting of a heavy chain variable region and a light chain variable region, wherein the first and second variable regions are not the same. In one embodiment, each first and second intracellular immunoglobulin subunit polypeptide also comprises a constant region, preferably an intracellular constant region, selected from the group consisting of a heavy chain constant region and a light chain constant region, wherein the first and second constant regions are not the same. Accordingly, through the association of one heavy chain and one light chain, an immunoglobulin molecule or immunoglobulin fragment, preferably an intracellular immunoglobulin molecule or fragment is formed

Also in certain preferred embodiments in the context of an intracellular immunoglobulin fragment, a single-chain fragment comprises an immunoglobulin variable region selected from the group consisting of a heavy chain variable region and a light chain variable region, and preferably comprises both variable regions. If the intracellular immunoglobulin fragment comprises both a heavy chain variable region and a light chain variable region, they may be directly joined (i.e., they have no peptide or other linker), or they may be joined by another means. If they are joined by other means, they may be joined directly or by a disulfide bond formed during expression or by a peptide linker, as discussed below. Accordingly, through the association of the heavy chain variable region and the light chain variable region, a CDR is formed. The heavy chain variable region and light chain variable region of a single-chain fragment may associate with one another or the heavy chain variable region of one single-chain fragment may associate with a light chain variable region of another single-chain fragment, and vise versa, depending on the type of linker. In one embodiment, the single-chain fragment also comprises a constant region selected from the group consisting of a heavy chain constant region, or a domain thereof, and a light chain constant region, or a domain thereof. Two single-chain fragments may associate with one another via their constant regions.

As mentioned above, in certain embodiments, the polynucleotide encoding the light chain variable region and heavy chain variable region of the single-chain fragment encode a linker. The single-chain fragment preferably properly folds even under the reducing conditions sometimes encountered intracellularly. The single-chain fragment may comprise a single polypeptide with the sequence V_{H}-linker-V_{L} or V_{L}-linker-V_{H}. In some embodiments, the linker is chosen to permit the heavy chain and light chain of a single polypeptide to bind together in their proper conformational orientation. See for example, Huston, J. S., et al, Methods in Enzym. 203:46-121 (1991). Thus, in these embodiments, the linker should be able to span the 3.5 nm distance between its points of fusion to the variable domains without distortion of the native Fv conformation. In these embodiments, the amino acid residues constituting the linker are such that it can span this distance and should be 5 amino acids or longer. Single-chain fragments with a linker of 5 amino acids form are found in monomer and predominantly dimer form. Preferably, the linker should be at least about 10 or at least about 15 residues in length. In other embodiments, the linker length is chosen to promote the formation of scFv tetramers (tetrabodies), and is 1 amino acid in length. In some embodiments, the variable regions are directly linked (i.e., the single-chain fragment contains no peptide linker) to promote the formation of scFv trimers (triabodies). These variations are well known in the art. (See, for example, Chames and Baty, FEMS Microbiol. Letts. 189:1-8 (2000). The linker should not be so long it causes steric interference with the combining site. Thus, it preferably should be about 25 residues or less in length.

The amino acids of the peptide linker are preferably selected so that the linker is hydrophilic so it does not get buried into the antibody. The linker (Gly-Gly-Gly-Gly-Ser)₃ (SEQ ID NO:1) is a preferred linker that is widely applicable to many antibodies as it provides sufficient flexibility. Other linkers include Glu Ser Gly Arg Ser Gly Gly Gly Gly Ser Gly Gly Gly Gly Ser (SEQ ID NO:2), Glu Gly Lys Ser Ser Gly Ser Gly Ser Glu Ser Lys Ser Thr (SEQ ID NO:3), Glu Gly Lys Ser Ser Gly Ser Gly Ser Glu Ser Lys Ser Thr Gln (SEQ ID NO:4), Glu Gly Lys Ser Ser Gly Ser Gly Ser Glu Ser Lys Val Asp (SEQ ID NO:5), Gly Ser Thr Ser Gly Ser Gly Lys Ser Ser Glu Gly Lys Gly (SEQ ID NO:6), Lys Glu Ser Gly Ser Val Ser Ser Glu Gln Leu Ala Gln Phe Arg Ser Leu Asp (SEQ ID NO:7), and Glu Ser Gly Ser Val Ser Ser Glu Glu Leu Ala Phe Arg Ser Leu Asp (SEQ ID NO:8). Alternatively, a linker such as the (Gly-Gly-Gly-Gly-Ser)₃ (SEQ ID NO:1) linker, although any sequence can be used, is mutagenized or the amino acids in the linker are randomized, and using phage display vectors or the methods of the invention, antibodies with different linkers are screened or selected for the highest affinity or most modification of a given phenotype. Examples of shorter linkers include fragments of the above linkers, and examples of longer linkers include combinations of the linkers above, combinations of fragments of the linkers above, and combinations of the linkers above with fragments of the linkers above.

Preferably, the polynucleotide does not encode the normal leader sequence for the variable chains. It is preferable that the antibody does not encode a leader sequence. The nucleotides coding for the binding portion of the antibody preferably do not encode the antibody's secretory sequences (i.e. the sequences that cause the antibody to be secreted from the cell).

Also preferred are intracellular immunoglobulin subunit polypeptides which are variants or fragments of the above-described intracellular immunoglobulin subunit polypeptides. Any variants or fragments of an intracellular immunoglobulin or fragment thereof which directly or indirectly induce a predetermined modified phenotype are contemplated. For example, a polynucleotide encoding an intracellular immunoglobulin molecule or fragment thereof isolated by the methods of the invention may be cloned into another antibody-encoding polynucleotide to form an immunoglobulin variant-encoding polynucleotide. Such variants may include sequences which allow them to be attached to the host cell surface, e.g., through association with a naturally-occurring transmembrane domain, through a receptor-ligand interaction, or as a fusion with a heterologous transmembrane domain, or allow them to be secreted into the cell medium, or allow them to be targeted to a different subcellular compartment.

In those embodiments where the intracellular immunoglobulin subunit polypeptide or fragment comprises a heavy chain polypeptide, any immunoglobulin heavy chain or region or domain thereof from any animal species, is intended. Suitable and preferred immunoglobulin heavy chains are described herein. Immunoglobulin heavy chains from vertebrates such as birds, especially chickens, fish, and mammals are included, with mammalian immunoglobulin heavy chains being preferred. Examples of mammalian immunoglobulin heavy chains include human, mouse, dog, cat, horse, goat, rat, sheep, cow, pig, guinea pig, and hamster immunoglobulin heavy chains. Of these, human immunoglobulin heavy chains are particularly preferred. Also contemplated are hybrid immunoglobulin heavy chains comprising portions of heavy chains from one or more species, such as mouse/human hybrid immunoglobulin heavy chains, or “camelized” human immunoglobulin heavy chains. Of the human immunoglobulin heavy chains, preferably, an immunoglobulin heavy chain of the present invention is selected from the group consisting of a μ heavy chain, i.e., the heavy chain of an IgM immunoglobulin, a γ-1 heavy chain, i.e., the heavy chain of an IgG1 immunoglobulin, a γ-2 heavy chain, i.e., the heavy chain of an IgG2 immunoglobulin, a γ-3 heavy chain, i.e., the heavy chain of an IgG3 immunoglobulin, a γ-4 heavy chain, i.e., the heavy chain of an IgG4 immunoglobulin, an α-1 heavy chain, i.e., the heavy chain of an IgA1 immunoglobulin, an α-2 heavy chain, i.e., the heavy chain of an IgA2 immunoglobulin, and ε heavy chain, i.e., the heavy chain of an IgE immunoglobulin, and a δ heavy chain, i.e., the heavy chain of an IgD immunoglobulin. In preferred embodiments, the intracellular immunoglobulin subunit polypeptide or immunoglobulin fragment includes only a portion of an immunoglobulin heavy chain. For example, in preferred embodiments, the immunoglobulin heavy chains lack sequences necessary for secretion or expression on the outer surface of the cell membrane, such as a signal peptide and/or membrane-spanning domain. For example, in preferred embodiments, the intracellular immunoglobulin subunit polypeptide or intracellular immunoglobulin fragment includes only a domain or a combination of domains of an immunoglobulin heavy chain. In a particularly preferred embodiment, the immunoglobulin subunit polypeptide or immunoglobulin fragment both includes only a domain or a combination of domains and lacks sequences necessary for secretion or cell surface expression. In certain embodiments, the immunoglobulin heavy chains include membrane-bound forms of human μ, γ-1, γ-2, γ-3, γ-4, α-1, α-2, ε, and δ heavy chains. In these embodiments, especially preferred is a membrane bound form of the human μ heavy chain.

In those embodiments where the intracellular immunoglobulin subunit polypeptide or immunoglobulin fragment comprises a light chain polypeptide, any immunoglobulin light chain or region or domain thereof, from any animal species, is intended. Suitable and preferred immunoglobulin light chains are described herein. Immunoglobulin light chains from vertebrates such as birds, especially chickens, fish, and mammals are included, with mammalian immunoglobulin light chains being preferred. Examples of mammalian immunoglobulin light chains include human, mouse, dog, cat, horse, goat, rat, sheep, cow, pig, guinea pig, and hamster immunoglobulin light chains. Of these, human immunoglobulin light chains are particularly preferred. Also contemplated are hybrid immunoglobulin light chains comprising portions of light chains from one or more species, such as mouse/human hybrid immunoglobulin light chains. Preferred immunoglobulin light chains include human κ and λ light chains. For immunoglobulins, a pair of either light chain may associate with an identical pair of any of the heavy chains to produce an immunoglobulin molecule, with the characteristic H₂L₂ structure which is well understood by those of ordinary skill in the art. In preferred embodiments, the intracellular immunoglobulin subunit polypeptide or immunoglobulin fragment includes only a portion of an immunoglobulin light chain. For example, in preferred embodiments, the intracellular immunoglobulin light chains lack sequences necessary for secretion or expression on the outer surface of the cell membrane, such as a signal peptide. For example, in preferred embodiments, the intracellular immunoglobulin subunit polypeptide or immunoglobulin fragment includes only a variable domain or a combination of variable and constant domains of an immunoglobulin light chain. In a particularly preferred embodiment, the intracellular immunoglobulin subunit polypeptide or immunoglobulin fragment includes only a variable domain and lacks sequences necessary for secretion or cell surface expression.

According to a preferred aspect of the invention, each member of a first library of polynucleotides or a second library of polynucleotides, comprises (a) a first nucleic acid molecule encoding an immunoglobulin constant region common to all members of the library, and (b) a second nucleic acid molecule encoding an immunoglobulin variable region, where the second nucleic acid molecule is directly upstream of and in-frame with the first nucleic acid molecule.

Accordingly, an intracellular immunoglobulin subunit polypeptide encoded by a member of a library of polynucleotides of the present invention, i.e., an immunoglobulin light chain or an immunoglobulin heavy chain encoded by such a polynucleotide, preferably comprises an immunoglobulin constant region associated with an immunoglobulin variable region.

The constant region of a light chain encoded by the “first nucleic acid molecule,” comprises about half of the subunit polypeptide and is situated C-terminal, i.e., in the latter half of the light chain polypeptide. A light chain constant region, referred to herein as a C_{L} constant region, or, more specifically a Cκ constant region or a Cλ constant region, comprises about 110 amino acids held together in a “loop” by an interchain disulfide bond.

The constant region of a heavy chain encoded by the “first nucleic acid molecule” comprises three quarters or more of the subunit polypeptide, and is situated in the C-terminal, i.e., in the latter portion of the heavy chain polypeptide. The heavy chain constant region, referred herein as a C_{H} constant region, comprises either three or four peptide loops or “domains” of about 110 amino acid each enclosed by interchain disulfide bonds. More specifically, the heavy chain constant regions in human immunoglobulins include a Cμ constant region, a Cδ constant region, a Cγ constant region, a Cα constant region, and a Cε constant region. Cγ, Cα, and Cδ heavy chains each contain three constant region domains, referred to generally as C_{H}1, C_{H}2, and C_{H}3, while Cμ and Cε heavy chains contain four constant region domains, referred to generally as C_{H}1, C_{H}2, C_{H}3, and C_{H}4. Nucleic acid molecules encoding human immunoglobulin constant regions are readily obtained from cDNA libraries derived from, for example, human B cells or their precursors by methods such as PCR, which are well known to those of ordinary skill in the art and further, are disclosed in the Examples, herein.

Intracellular immunoglobulin subunit polypeptides of the present invention encoded by the “first or second nucleic acid molecule” (i.e., encoded by the first and second libraries) and single-chain fragments of the present invention encoded by libraries each comprise an immunoglobulin variable region. The library will contain a plurality, i.e., at least two, preferably at least 10, 100, 10³, 10⁴, 10⁵, 10⁶, 10⁷, 10⁸, 10⁹ 10¹⁰, 10¹¹, or 10¹² different variable regions. If the library contains a constant region, each polynucleotide comprises the same constant region. As is well known by those of ordinary skill in the art, a light chain variable region is encoded by rearranged nucleic acid molecules, each comprising a light chain V_{L} region, specifically a Vκ region or a Vλ region, and a light chain J region, specifically a Jκ region or a Jλ region. Similarly, a heavy chain variable region is encoded by rearranged nucleic acid molecules, each comprising a heavy chain V_{H} region, a D region and J region. These rearrangements take place at the DNA level upon cellular differentiation. Nucleic acid molecules encoding heavy and light chain variable regions may be derived, for example, by PCR from mature B cells and plasma cells which have terminally differentiated to express an antibody with specificity for a particular epitope. Furthermore, if antibodies to a specific antigen are desired, variable regions may be isolated from mature B cells and plasma cells of an animal who has been immunized with that antigen, and has thereby produced an expanded repertoire of antibody variable regions which interact with the antigen. Alternatively, if a more diverse library is desired, variable regions may be isolated from precursor cells, e.g., pre-B cells and immature B cells, which have undergone rearrangement of the immunoglobulin genes, but have not been exposed to antigen, either self or non-self. For example, variable regions might be isolated by PCR from normal human bone marrow pooled from multiple donors. Alternatively, variable regions may be synthetic, for example, made in the laboratory through generation of synthetic oligonucleotides, or may be derived through in vitro manipulations of germ line DNA resulting in rearrangements of the immunoglobulin genes.

Polynucleotides may be introduced into host cells by methods which are well known to those of ordinary skill in the art. Suitable and preferred introduction methods are disclosed herein. As is easily appreciated, introduction methods vary depending on the nature of the vector in which the polynucleotide libraries are constructed. For example, DNA plasmid vectors may be introduced into host cells, for example, by lipofection (such as with anionic liposomes (see, e.g., Felgner et al., 1987 Proc. Natl. Acad Sci. U.S.A. 84:7413 or cationic liposomes (see, e.g., Brigham, K. L. et al. Am. J Med Sci. 298(4): 278-2821(1989); U.S. Pat. No.4,897,355 (Eppstein, et al.)), by electroporation, by calcium phosphate precipitation (see generally, Sambrook et al., Molecular Cloning: A Laboratory Manual, 2nd Ed., Cold Spring Harbor Laboratory, Cold Spring Harbor, N.Y., 1989), by protoplast fusion, by spheroplast fusion, or by the DEAE dextran method (Sussman et al., Cell. Biol. 4:1641-1643 (1984)).

When the selected method is lipofection, the nucleic acid can be complexed with a cationic liposome, such as DOTMA:DOPE, DOTMA, DOPE, DC-cholesterol, DOTAP, Transfectam® (Promega), Tfx® (Promega), LipoTAXI™ (Stratagene), PerFect Lipid™ (Invitrogen), SuperFect™ (Qiagen). When the nucleic acid is transfected via an anionic liposome, the anionic liposome can encapsulate the nucleic acid. Preferably, DNA is introduced by liposome-mediated transfection using the manufacturer's protocol (such as for Lipofectamine; Life Technologies Incorporated).

Where the plasmid is a virus vector, introduction into host cells is most conveniently carried out by standard infection. However, in many cases viral nucleic acids may be introduced into cells by any of the methods described above, and the viral nucleic acid is “infectious,” i.e., introduction of the viral nucleic acid into the cell, without more, is sufficient to allow the cell to produce progeny virus particles. It is noted, however, that certain virus nucleic acids, for example, poxvirus nucleic acids, are not infectious, and therefore must be introduced with additional elements provided, for example, by a virus particle enclosing the viral nucleic acid, by a cell which has been engineered to produce required viral elements, or by a helper virus.

If there are two libraries of polynucleotides, they may be introduced into host cells in any order, or simultaneously. For example, if both the first and second libraries of polynucleotides are constructed in virus vectors, whether infectious or inactivated, the vectors may be introduced by simultaneous infection as a mixture, or may be introduced in consecutive infections. If one library is constructed in a virus vector, and the other is constructed in a plasmid vector, introduction might be carried out most conveniently by introduction of one library before the other. For example, in a preferred embodiment, the cells are first infected with the virus library, and the plasmid library is subsequently transfected into the infected cells.

Following introduction into the host cells of the libraries of polynucleotides, expression of intracellular immunoglobulin molecules, or fragments thereof, is permitted to occur. By “permitting expression” is meant allowing the vectors which have been introduced into the host cells to undergo transcription and translation of the intracellular immunoglobulin subunit polypeptides, preferably allowing the host cells to transport assembled intracellular immunoglobulin molecules, or fragments thereof to the appropriate cellular location. Typically, permitting expression requires incubating the host cells into which the polynucleotides have been introduced under suitable conditions to allow expression. Those conditions, and the time required to allow expression will vary based on the choice of host cell and the choice of vectors, as is well known by those of ordinary skill in the art.

Heterologous sequences. Each first and second polynucleotides encoding intracellular immunoglobulin subunit polypeptides, or fragments thereof, may further comprise a heterologous sequence upstream of or downstream from the sequence encoding the intracellular immunoglobulin molecules, or fragments thereof. Likewise, the polynucleotides encoding single-chain intracellular immunoglobulins may further comprise a heterologous sequence upstream of or downstream from the sequence encoding the single-chain intracellular immunoglobulin. As such, it is noted that the term “heterologous sequence” may include a heterologous polynucleotide sequence, may be located upstream or downstream of the polynucleotide sequence encoding the intracellular immunoglobulin molecule, or fragment thereof, and the heterologous sequence may be in operable association with the polynucleotide sequence encoding the intracellular immunoglobulin molecule, or fragment thereof. Furthermore, the heterologous polynucleotide sequence may encode a heterologous polypeptide, which may be fused, either upstream or downstream or at both ends, to the intracellular immunoglobulin molecule, or fragment thereof. Generally, if a “heterologous polynucleotide” is associated with a library of polynucleotides encoding an intracellular immunoglobulin subunit polypeptide or an intracellular single-chain immunoglobulin, each individual member of the library will comprise the same heterologous polynucleotide.

Some preferred heterologous sequences are disclosed in U.S. Pat. No. 6,153,380, which is incorporated herein by reference in its entirety. For example, in a preferred embodiment, the Ig molecules or Ig fragments comprise a targeting sequence capable of constitutively localizing the intracellular immunoglobulin molecule, or fragment thereof, to a predetermined cellular locale, including subcellular locations such as the golgi, endoplasmic reticulum, nucleus, nucleoli, nuclear membrane, mitochondria, chloroplast, secretory vesicles, lysosome, and cellular membrane.

In a preferred embodiment, the targeting sequence is a nuclear localization signal (NLS). NLSs are generally short, positively charged (basic) domains that serve to direct the entire protein in which they occur to the cell's nucleus. Numerous NLS amino acid sequences have been reported including single basic NLS's such as that of the SV40 (monkey virus) large T Antigen (Pro Lys Lys Lys Arg Lys Val) (SEQ ID NO:10), Kalderon (1984), et al., Cell, 39:499 509; the human retionic acid receptor-β nuclear localization signal (ARRRRP) (SEQ ID NO:11); NFKB p50 (EEVQRKRQKL (SEQ ID NO:12); Ghosh et al., Cell 62:1019 (1990); NFKB p65 (EEKRKRTYE (SEQ ID NO:13); Nolan et al., Cell 64;961 (1991); and others (see for example Boulikas, J. Cell. Biochem. 55(l): 32-58 (1994)) and double basic NLS's exemplified by that of the Xenopus protein, nucleoplasmin (Ala Val LysArg ProAla AlaThr Lys Lys Ala Gly Gln Ala Lys Lys Lys Lys Leu Asp) (SEQ ID NO:14), Dingwall, et al., Cell, 30:449-458, 1982 and Dingwall, et al., J. Cell Biol., 107:641-849; 1988). Numerous localization studies have demonstrated that NLSs incorporated in synthetic peptides or grafted onto reporter proteins not normally targeted to the cell nucleus cause these peptides and reporter proteins to be concentrated in the nucleus. See, for example, Dingwall, and Laskey Ann, Rev. Cell Biol., 2:367-390, 1986; Bonnerof, et al., Proc. Natl. Acad. Sci. USA, 84:6795-6799, 1987; Galileo, et al., Proc. Natl. Acad. Sci. USA, 87:458-462, 1990.

In a preferred embodiment, the targeting sequence is a membrane anchoring signal sequence. This is useful since many parasites and pathogens bind to the membrane, in addition to the fact that many intracellular events originate at the plasma membrane. Thus, membrane bound libraries are useful for both the identification of important elements in these processes as well as for the discovery of effective inhibitors. The invention provides methods for presenting the intracellular immunoglobulin molecule, or fragment thereof, extracellularly or in the cytoplasmic space. For extracellular presentation, a membrane anchoring region is provided at the carboxyl terminus of the Ig or Ig fragment. The Ig or Ig fragment is expressed on the cell surface and presented to the extracellular space, such that it can bind to other surface molecules (affecting their function) or molecules present in the extracellular medium. The binding of such molecules could inhibit or confer function on the cells expressing an Ig or Ig fragment that binds the molecule. The cytoplasmic region could be neutral or could contain a domain that, when the Ig or Ig fragment is bound, confers a function on the cells (activation of a kinase, phosphatase, binding of other cellular components to effect function). Similarly, the Ig or Ig fragment could be contained within a cytoplasmic region, and the transmembrane region and extracellular region remain constant or have a defined function.

Membrane-anchoring sequences are well known in the art and are based on the genetic geometry of mammalian transmembrane molecules. Peptides are introduced into the membrane based on a signal sequence (designated herein as ssTM) and require a hydrophobic transmembrane domain (herein TM). The transmembrane proteins are introduced into the membrane such that the regions encoded 3′ of the transmembrane domain are intracellular and the sequences 5′ become extracellular. In preferred embodiment, the transmembrane domains are placed 5′ of the Ig or Ig fragment they will serve to anchor it as an intracellular domain. ssTMs and TMs are known for a wide variety of membrane bound proteins, and these sequences may be used accordingly, either as pairs from a particular protein or with each component being taken from a different protein, or alternatively, the sequences may be synthetic, and derived entirely from consensus as artificial delivery domains.

As will be appreciated by those in the art, membrane anchoring sequences, including both ssTM and TM, are known for a wide variety of proteins and any of these may be used. Particularly preferred membrane-anchoring sequences include, but are not limited to, those derived from CD8, ICAM-2, IL-8R, CD4 and LFA-1.

Useful sequences include sequences from: 1) class I integral membrane proteins such as IL-2 receptor beta-chain (residues 1-26 are the signal sequence, 241-265 are the transmembrane residues; see Hatakeyama et al, Science 244:551 (1989) and von Heijne et al, Eur. J. Biochem. 174:671 (1988)) and insulin receptor beta-chain (residues 1-27 are the signal, 957-959, are the transmembrane domain and 960-1382 are the cytoplasmic domain; see Hatakeyama supra, and Ebina et al., Cell 40:747 (1985)); 2) class II integral membrane proteins such as neutral endopeptidase (residues 29-51 are the transmembrane domain, 2-28 are the cytoplasmic domain; see Malfroy et al., Biochem. Biophys. Res. Commun. 144:59 (1987)); 3) type III proteins such as human cytochrome P450 NF25 (Hatakeyama, supra); and 4) type IV proteins such as human P-glycoprotein (Hatakeyama, supra). Particularly preferred are CD8 and ICAM-2. For example, the signal sequences from CD8 and ICAM-2 lie at the extreme 5′ end of the transcript. These consist of the amino acids 1-32 in the case of CD8 (MASPLTRFLSLNLLLLGESILGSGEAKPQAP (SEQ ID NO:15); Nakauchi et al., PNAS USA 82:5126 (1985) and 1-21 in the case of ICAM-2 (MSSFGYRTLTVALFTLICCPG (SEQ ID NO:16); Staunton et al., Nature (London) 339:61 (1989)). These leader sequences deliver the construct to the membrane while the hydrophobic transmembrane domains, placed 5′ or 3′ of the Ig or Ig fragment, serve to anchor the construct in the membrane. These transmembrane domains are encompassed by amino acids 145-195 from CD8 (PQRPEDCRPRGSVKGTGLDFACDIYIWAPLAGICVALLLSLIITLICYHSR (SEQ ID NO:18); Nakauchi, supra) and 224-256 from ICAM-2 (MVIIVTVVSVLLSLFVTSVLLCFIFGQHLRQQR (SEQ ID NO:19); Staunton, supra).

Alternatively, membrane anchoring sequences include the GPI anchor, which results in a covalent bond between the molecule and the lipid bilayer via a glycosyl-phosphatidylinositol bond for example in DAF (PNKGSGTTSGTTRLLSGHTCFTLTGLLGTLVTMGLLT (SEQ ID NO:20); see Homans et al., Nature 333(6170):269-72 (1988), and Moran et al., J. Biol. Chem. 266:1250 (1991)). In order to do this, the GPI sequence from Thy-1 can be cassetted 3′ of the Ig or Ig fragment in place of a transmembrane sequence.

Similarly, myristylation sequences can serve as membrane anchoring sequences. It is known that the myristylation of c-src recruits it to the plasma membrane. This is a simple and effective method of membrane localization, given that the first 14 amino acids of the protein are solely responsible for this function: MGSSKSKPKDPSQR (SEQ ID NO:17) (see Cross et al., Mol. Cell. Biol. 4(9) 1834(1984); Spencer et al., Science 262:1019 1024 (1993). This motif has already been shown to be effective in the localization of reporter genes and can be used to anchor the zeta chain of the TCR. This motif is placed 5′ of the Ig or Ig fragment in order to localize the construct to the plasma membrane. Other modifications such as palmitoylation can be used to anchor constructs in the plasma membrane; for example, palmitoylation sequences from the G protein-coupled receptor kinase GRK6 sequence (LLQRLFSRQDCCGNCSDSEEELPTRL (SEQ ID NO:21); Stoffel et al, J. Biol. Chem 269:27791 (1994)); from rhodopsin (KQFRNCMLTSLCCGKNPLGD (SEQ ID NO:22); Barnstable et al., J. Mol. Neurosci. 5(3):207 (1994)); and the p21 II-ras 1 protein (LNPPDESGPGCMSCKCVLS (SEQ ID NO:23); Capon et al., Nature 302:33 (1983)).

In a preferred embodiment, the targeting sequence is a lysozomal targeting sequence, including, for example, a lysosomal degradation sequence such as Lamp-2 (KFERQ (SEQ ID NO:24); Dice, Ann. N.Y. Acad. Sci. 674:58 (1992); or lysosomal membrane sequences from Lamp-I (MLIPIAGFFALAGLVLIVLIAYLIGRKRSHAGYQTI (SEQ ID NO:25), Uthayakumar et al., Cell. Mol. Biol. Res. 41:405 (1995)) or Lamp-2 (LVPIAVGAALAGVLILVLLAYFIGLKHHHAGYEQF (SEQ ID NO:26), Konecki et al., Biochem. Biophys. Res. Comm. 205:1-5 (1994), both of which show the transmembrane domains in italics and the cytoplasmic targeting signal underlined.

Alternatively, the targeting sequence maybe a mitochondrial localization sequence, including mitochondrial matrix sequences (e.g. yeast alcohol dehydrogenase III; MLRTSSLFTRRVQPSLFSRNILRLQST (SEQ ID NO:27); Schatz, Eur. J. Biochem. 165:1-6 (1987)); mitochondrial inner membrane sequences (yeast cytochrome c oxidase subunit IV, MLSLRQSIRFFKPATRTLCSSRYLL (SEQ ID NO:28); Schatz, supra); mitochondrial intermembrame space sequences (yeast cytochrome c1; M F S M L S K R W A Q R - TLSKSFYSTATGAASKSGKLTQKLVTAGVMAGITASTLLYADSLTAEA MTA (SEQ ID NO:29); Schatz, supra) or mitochondrial outer membrane sequences (yeast 70 kD outer membrane protein; MKSFITRNKTAILATVAATGTAIGAYYYYNQLQQQQQRGKK (SEQ ID NO:30); Schatz, supra).

The target sequence may also be an endoplasmic reticulum sequence, including the sequences from calreticulin (KDEL (SEQ ID NO:31); Pelham, Royal Society London Transactions B; 1-10 (1992)) or adenovirus E3/19K protein (LYLSRRSFIDEKKMP (SEQ ID NO:32); Jackson et al., EMBO J. 9:3153 (1990).

Furthermore, targeting sequences also include peroxisome sequences (for example, the peroxisome matrix sequence from Luciferase; SYL; Keller et al., PNAS USA 4:3264 (1987)); farnesylation sequences (for example, P21 H-ras 1; LNPPDESGPGCMSCKCVLS (SEQ ID NO:33), Capon, supra); gera-nylgeranylation sequences (for example, protein rab-5A; LTEPTQPTRNQCCSN (SEQ ID NO:34); Farnsworth, PNAS USA 91:11963 (1994)); or destruction sequences (cyclin B1; RTALGDIGN (SEQ ID NO:35); Klotzbucher et al., EMBO J. 1:3053 (1996)).

In one embodiment, the targeting sequence is a secretory signal sequence capable of effecting the secretion of the Ig or Ig fragment. This approach is particularly suitable for synthesizing intracellular immunoglobulin molecules, or fragments thereof, from polynucleotides isolated by the methods of the invention, when the isolated intracellular immunoglobulin molecule or fragment thereof is to be used in further experiments, for example, to isolate or characterize the target epitope recognized by the intracellular immunoglobulin or fragment thereof, or for therapeutic purposes. There are a large number of known secretory signal sequences which are placed 5′ to the intracellular immunoglobulin or fragment thereof region, and are cleaved to effect secretion into the extracellular space.

Secretory signal sequences and their transferability to unrelated proteins are well known, e.g., Silhavy, et al (1985) Microbiol. Rev. 49, 398-418.

Suitable secretory sequences are known, including signals from IL-2 (MYRMQLLSCIALSLALVTNS (SEQ ID NO:36); Villinger et al., J. Immunol. 1 5 5: 3 9 4 6 ( 1 9 9 5 )), growth hormone (MATGSRTSLLLAFGLLCLPWLQEGSAFPT (SEQ ID NO:37); Roskam et al., Nucleic Acids Res. 7:30 (1979)); preproinsulin (MALWMRLLPLLALLALWGPDPAAA FVN (SEQ ID NO:38); Bell et al., Nature 284:26 (1980)); and influenza HA protein (MKAKLLVLLYAFVAGDQI (SEQ ID NO:39); Sekiwawa et al., PNAS 80:3563)), with cleavage between the non-underlined-underlined junction. A particularly preferred secretory signal sequence is the signal leader sequence from the secreted cytokine IL4, which comprises the first 24 amino acids of IL-4 as follows: MGLTSQLLPPLFFLLACAGNFVHG (SEQ ID NO:40).

In a preferred embodiment, the heterologous polypeptide is a rescue sequence. A rescue sequence is a sequence which may be used to purify or isolate either the intracellular immunoglobulin molecule, or fragment thereof, or the polynucleotide encoding it. Thus, for example, peptide rescue sequences include purification sequences such as the 6-His tag for use with Ni affinity columns and epitope tages for detection, immunoprecipitation, or FACS (fluorescence-activated cell sorting). Suitable epitope tags include myc (for use with commercially available 9E10 antibody), the BSP biotinylation target sequence (a short peptide sequence that binds to bacterial enzyme BirA), influenza tags (for example, those that are derived from nucleoprotein or hemagglutinin proteins of influenza virus), LacZ (β-galactosidase) or active fragments thereof, and GST (glutathione S-transferase) or active fragment thereof. Suitable epitope tags also include any detectable fragments of any known epitope tags.

In a preferred embodiment, combinations of heterologous polypeptides are used. Thus, for example, any number of combinations of targeting sequences, secretory sequences, rescue sequences, and stability sequences may be used, with or without linker sequences. One can cassette in various fusion polynucleotides encoding heterologous polypeptides 5′ and 3 of the intracellular immunoglobulin molecule, or fragment thereof-encoding polynucleotide. Table 1 outlines some of the possible combinations as follows. Using Ig as the intracellular immunoglobulin molecule, or fragment thereof, and representing each targeting sequence by another letter, (e.g. N for nuclear localization sequence) each construct can be named as a string of representative letters reading N-terminal to C-terminal as protein, such as NIg or if cloned downstream of the intracellular immunoglobulin or fragment thereof region, IgN. As implied here, the heterologous sequences are cloned as cassettes into sites on either side of the intracellular immunoglobulin molecule, or fragment thereof. C is for cytoplasmic (e.g. no localization sequence), E is a rescue sequence such as the myc epitope, G is a linker sequence (G10 is a glycine-serine chain of 10 amino acids, and G20 is a glycine-serine chain of 20 amino acids), M is a myristylation sequence, N is a nuclear localization sequence, ssTM is the signal sequence for a transmembrane anchoring sequence, TM is the transmembrane anchoring sequence, GPI is a GPI membrane anchor sequence; S is a secretory signal sequence, etc. As will be appreciated by those in the art, any number of combinations can be made, in addition to those listed below.

TABLE 1

| | |
|---|---|
| cytoplasmic | C Ig |
| | C E Ig |
| | C Ig E |
| secreted | S Ig |
| | S E Ig |
| | S Ig E |
| myristylated | M Ig |
| | M E Ig |
| | M GE20 Ig |
| transmembrane | ssTM Ig |
| (intracellular) | ssTM Ig TM |
| | ssTM Ig E TM |
| | ssTM Ig G20 E TM |
| | ss TM Ig E |
| transmembrane (GPI linked) | ssTM Ig G E TM |
| nuclear localization | M E Ig |
| | N Ig E |

As will be appreciated by those in the art, these modules of sequences can be used in a large number of combinations and variations.

The localization signals can be located anywhere on the antibody so long as the signal is exposed in the antibody and its placement does not disrupt the binding ability of the antibody or the ability of the antibody to interfere with the antigen thus causing or “inducing” a modified phenotype. For example, it can be placed at the carboxy or amino terminus or even on the linker between the heavy and light chain of a single-chain fragment, providing it satisfies the above conditions.

Additional heterologous sequences include the following from WO 94/02610 and WO 99/14353, the disclosures of which are incorporated herein by reference in their entireties: For example, signals such as Lys Asp Glu Leu (SEQ ID NO:41) [Munro, et al., Cell 48:899-907 (1987)] Asp Asp Glu Leu (SEQ ID NO:42), Asp Glu Glu Leu (SEQ ID NO:43), Gln Glu Asp Leu (SEQ ID NO:44) and Arg Asp Glu Leu (SEQ ID NO:45) [Hangejorden, et al., J. Biol. Chem. 266:6015 (1991), for the endoplasmic reticulum; Pro Lys Lys Lys Arg Lys Val (SEQ ID NO:46) [Lanford, et al. Cell 46:575 (1986)] Pro Gln Lys Lys Ile Lys Ser (SEQ ID NO:47) [Stanton, L. W., et al., Proc. Natl. Acad. Sci USA 83:1772 (1986); Gln Pro Lys Lys Pro (SEQ ID NO:48) [Harlow, et al., Mol. Cell Biol. 5:1605 (1985)], Arg Lys Lys Arg (SEQ ID NO:49), for the nucleus; and Arg Lys Lys Arg Arg Gln Arg Arg Arg Ala His Gln (SEQ ID NO:50), [Seomi, et al., J. Virology 64:1803 (1990)], Arg Gln Ala Arg Arg Asn Arg Arg Arg Arg Trp Arg Glu Arg Gln Arg (SEQ ID NO:51) [Kubota, et al., Biochem. and Biophys, Res. Comm. 162:963 (1989)], Met Pro Leu Thr Arg Arg Arg Pro Ala Ala Ser Gln Ala Leu Ala Pro Pro Thr Pro (SEQ ID NO:52) [Siomi, et al., Cell 55:197 (1988)] for the nucleolar region; Met Asp Asp Gln Arg Asp Leu Ile Ser Asn Asn Glu Gln Leu Pro (SEQ ID NO:53), [Bakke, et al., Cell 63:707-716 (1990)] for the endosomal compartment. See, Letoumeur, et al., Cell 69:1183 (1992) for targeting liposomes. Myristolation sequences can be used to direct the antibody to the plasma membrane. In addition, as shown in Table 2 below, myristoylation sequences can be used to direct the antibodies to different subcellular locations such as the nuclear region. Localization sequences may also be used to direct antibodies to organelles, such as the mitochondria and the Golgi apparatus. The sequence Met Leu Phe Asn Leu Arg Xaa Xaa Leu Asn Asn Ala Ala Phe Arg His Gly His Asn Phe Met Val Arg Asn Phe Arg Cys Gly Gln Pro Leu Xaa (SEQ ID NO:54) can be used to direct the antibody to the mitochondrial matrix, (Pugsley, supra). See, Tang, et al., J. Biol. Chem. 207:10122, for localization of proteins to the Golgi apparatus.

TABLE 2

| | | | |
|---|---|---|---|
| AMINO- | | | |
| TERMINAL | SUBCELLULAR | | |
| SEQUENCE | LOCATION** | PROTEIN | REFERENCE |

| | | | |
|---|---|---|---|
| GCVCSSNP | PM | p56^{USTRATCK} | Marchildon, et al, Proc. |
| (SEQ ID | | | Natl. Acad. Sci. USA |
| NO:55) | | | 81:7679-7682 (1984) |
| | | | Voronova, et al. Mol. |
| | | | Cell. Biol. 4:2705-2713 |
| | | | (1984) |
| GQTVTTPL | PM | Mul. V gag | Henderson, et al, Proc. |
| (SEQ ID | | | Natl. Acad. Sci. USA |
| NO:56) | | | 80:339-343 (1987) |
| GQELSQHE | PM | M-PMV gag | Rhee, et al., J Virol. |
| (SEQ ID | | | 61:1045-1053 (1987) |
| NO:57) | | | |
| | | | Schultz, et al., J. Virol. |
| | | | 46:355-361 (1983) |
| GNSPSYNP | PM | BLV gag | Schultz, et al., J. Virol. |
| (SEQ ID | | | 133:431-437 (1984) |
| NO:58) | | | |
| GVSGSKG | PM | MMTV gag | Schultz et al., supra |
| Q (SEQ ID | | | |
| NO:59) | | | |
| GQTITTPL | PM | FCL. V gag | Schultz et al., supra |
| (SEQ ID | | | |
| NO:60) | | | |
| GQTLTTPL | PM | BaEV gag | Schultz et al., supra |
| (SEQ ID | | | |
| NO:61) | | | |
| GQIFSRSA | PM | HTLV-I gag | Ootsuyama, et al., Jpn |
| (SEQ ID | | | J. Cancer Res. 76:1132- |
| NO:62) | | | 1135 (1985) |
| GQIHGLSP | PM | HTLV-II gag | Ootsuyama, et al., |
| (SEQ ID | | | supra |
| NO:63) | | | |
| GARASVLS | PM | HIV (HTLV- | Ratner, et al., Nature |
| (SEQ ID | | III) gag | 313:277-284 (1985) |
| NO:64) | | | |
| GCTLSAEE | PM | bovine brain | Schultz, et al., |
| (SEQ ID | | Gₒ α-subunit | Biochem. Biophys. Res. |
| NO:65) | | | Commun. 146:1234- |
| | | | 1239 (1987) |
| GQNLSTSN | ER | Hepatitis B | Persing, et al., J. Virol. |
| (SEQ ID | | Virus pre-S1 | 61:1672-1677 (1987) |
| NO:66) | | | |
| GAALTILV | N | Polyoma | Streuli, et al., Nature |
| (SEQ ID | | Virus VP2 | 326:619-622 (1987) |
| NO:67) | | | |
| GAALTLLG | N | SV40 Virus | Streuli, et al., supra |
| (SEQ ID | | VP2 | |
| NO:68) | | | |
| GAQVSSQ | S,ER | Poliovirus | Chow, et al., Nature |
| K (SEQ ID | | VP4 | 327:482-486 (1987) |
| NO:69) | | | |
| | | | Paul, et al., Proc. Natl. |
| | | | Acad. Sci. USA |
| | | | 84:7827-7831 (1987) |
| GAQLSRNT | S,ER | Bovine | Paul, et al., supra |
| (SEQ ID | | Enterovirus | |
| NO:70) | | VP4 | |
| GNAAAAK | G,S,N,C | cAMP- | Carr, et al., Proc. Natl. |
| K | | dependent | Acad. Sci. USA |
| (SEQ ID | | kinase | 79:6128-6131 (1982) |
| NO:71) | | | |
| GNEASYPL | S,C | calcincurin B | Aitken, et al. FEBS |
| (SEQ ID | | | Lett. |
| NO:72) | | | 150: 314-318 (1982) |
| GSSKSKPK | PM,C | P60^{SFC} | Schultz, et al., Science |
| (SEQ ID | | | 227:427-429 (1985) |
| NO:73) | | | |

**Abbreviations are PM, plasma membranes, G, Golgi; N, Nuclear; C, Cytoskeleton; S, cytoplasm (soluble); M, membrane.
Additional heterologous sequences may be found in Example 1 and Persic, et al., Gene 187:1-8 (1997).

Antigens. As used herein, an “antigen” is any molecule that can specifically bind to an intracellular immunoglobulin molecule, or fragment thereof, or that an intracellular immunoglobulin or fragment thereof interferes with to induce a predetermined modified phenotype in a eukaryotic host cell. By “specifically bind” is meant that the antigen binds to the CDR of the antibody. The portion of the antigen which specifically interacts with the CDR is an “epitope,” or an “antigenic determinant.” An antigen may comprise a single epitope, but typically, an antigen comprises at least two epitopes, and can include any number of epitopes, depending on the size, conformation, and type of antigen. Almost any kind of biologic molecule can serve as an antigen, for example, intermediate metabolites, sugars, lipids, autacoids, and hormones as well as macromolecules such as complex carbohydrates, phospholipids, nucleic acids such as RNA and DNA, and proteins. The skilled artisan can generate antibodies that will interfere with and/or bind both the small molecules and macromolecules. For example, with small molecules one commonly attaches the small molecule (sometimes referred to as a hapten) to a macromolecule (sometimes referred to as a carrier) before immunization. The hapten-carrier complex acts as an immunogen. Thus antibodies that will interfere with and/or bind to a wide range of targets are known. The preferred target molecules include proteins, RNA, DNA and haptens. More preferably, the targets are proteins, RNA and DNA. Still more preferably, the target is a protein.

Antigens are typically peptides or polypeptides, but can be any molecule or compound. For example, an organic compound, e.g., dinitrophenol or DNP, a nucleic acid, a carbohydrate, or a mixture of any of these compounds either with or without a peptide or polypeptide can be a suitable antigen. The minimum size of a peptide or polypeptide epitope is thought to be about four to five amino acids. Peptide or polypeptide epitopes preferably contain at least seven, more preferably at least nine and most preferably between at least about 15 to about 30 amino acids. Since a CDR can recognize an antigenic peptide or polypeptide in its tertiary form, the amino acids comprising an epitope need not be contiguous, and in some cases, may not even be on the same peptide chain. In the present invention, peptide or polypeptide antigens preferably contain a sequence of at least 4, at least 5, at least 6, at least 7, more preferably at least 8, at least 9, at least 10, at least 15, at least 20, at least 25, and, most preferably, between about 15 to about 30 amino acids. Preferred peptides or polypeptides comprising, or alternatively consisting of, antigenic epitopes are at least 10, 15, 20, 25, 30, 35, 40, 45, 50, 55, 60, 65, 70, 75, 80, 85, 90, 95, or 100 amino acid residues in length. The antigen may be in any form and may be free, for example dissolved in a solution, or may be attached to any substrate. Suitable and preferred substrates are disclosed herein.

It is to be understood that intracellular immunoglobulin molecules specific for any antigen may be produced according to the methods of the present invention. Preferred antigens are “self” antigens, i.e., antigens derived from the same species as the immunoglobulin molecules produced. As an example, it might be desired to produce human antibodies directed to human antigens such as, but not limited to, a p53 antigen, a ATF-2 antigen, a CEA antigen, a GM2 antigen, a Tn antigen, an sTn antigen, a Thompson-Friedenreich antigen (TF), a Globo H antigen, an Le(y) antigen, a MUC1 antigen, a MUC2 antigen, a MUC3 antigen, a MUC4 antigen, a MUC5AC antigen, a MUC5B antigen, a MUC7 antigen, a carcinoembryonic antigen, a beta chain of human chorionic gonadotropin (hCG beta) antigen, a HER2/neu antigen, a PSMA antigen, a EGFRvIII antigen, a KSA antigen, a PSA antigen, a PSCA antigen, a GP100 antigen, a MAGE 1 antigen, a MAGE 2 antigen, a TRP 1 antigen, a TRP 2 antigen, and a tyrosinase antigen. Other desired “self” antigens include, but are not limited to, cytokines, receptors, ligands, glycoproteins, and hormones.

It is also contemplated to produce antibodies directed to antigens encoded by infectious agents. Examples of such antigens include, but are not limited to, bacterial antigens, viral antigens, parasite antigens, and fungal antigens. Examples of viral antigens include, but are not limited to, adenovirus antigens, alphavirus antigens, calicivirus antigens, e.g., a calicivirus capsid antigen, coronavirus antigens, distemper virus antigens, Ebola virus antigens, enterovirus antigens, flavivirus antigens, hepatitis virus (A-E) antigens, e.g., a hepatitis B core or surface antigen, herpesvirus antigens, e.g., a herpes simplex virus or varicella zoster virus glycoprotein antigen, immunodeficiency virus antigens, e.g., a human immunodeficiency virus envelope or protease antigen, infectious peritonitis virus antigens, influenza virus antigens, e.g., an influenza A hemagglutinin or neuraminidase antigen, leukemia virus antigens, Marburg virus antigens, oncogenic virus antigens, orthomyxovirus antigens, papilloma virus antigens, parainfluenza virus antigens, e.g., hemagglutinin/neuraminidase antigens, paramyxovirus antigens, parvovirus antigens, pestivirus antigens, picorna virus antigens, e.g., a poliovirus capsid antigen, rabies virus antigens, e.g., a rabies virus glycoprotein G antigen, reovirus antigens, retrovirus antigens, rotavirus antigens, as well as other cancer-causing or cancer-related virus antigens.

Examples of bacterial antigens include, but are not limited to, Actinomyces, antigens Bacillus antigens, Bacteroides antigens, Bordetella antigens, Bartonella antigens, Borrelia antigens, e.g., a B. bergdorferi OspA antigen, Brucella antigens, Campylobacter antigens, Capnocytophaga antigens, Chlamydia antigens, Clostridium antigens, Corynebacterium antigens, Coxiella antigens, Dermatophilus antigens, Enterococcus antigens, Ehrlichia antigens, Escherichia antigens, Francisella antigens, Fusobacterium antigens, Haemobartonella antigens, Haemophilus antigens, e.g., H. influenzae type b outer membrane protein antigens, Helicobacter antigens, Klebsiella antigens, L-form bacteria antigens, Leptospira antigens, Listeria antigens, Mycobacteria antigens, Mycoplasma antigens, Neisseria antigens, Neorickettsia antigens, Nocardia antigens, Pasteurella antigens, Peptococcus antigens, Peptostreptococcus antigens, Pneumococcus antigens, Proteus antigens, Pseudomonas antigens, Rickettsia antigens, Rochalimaea antigens, Salmonella antigens, Shigella antigens, Staphylococcus antigens, Streptococcus antigens, e.g., S. pyogenes M protein antigens, Treponema antigens, and Yersinia antigens, e.g., YpestisF1 and V antigens.

Examples of fungal antigens include, but are not limited to, Absidia antigens, Acremonium antigens, Alternaria antigens, Aspergillus antigens, Basidiobolus antigens, Bipolaris antigens, Blastomyces antigens, Candida antigens, Coccidioides antigens, Conidiobolus antigens, Cryptococcus antigens, Curvalaria antigens, Epidermophyton antigens, Exophiala antigens, Geotrichum antigens, Histoplasma antigens, Madurella antigens, Malassezia antigens, Microsporum antigens, Moniliella antigens, Mortierella antigens, Mucor antigens, Paecilomyces antigens, Penicillium antigens, Phialemonium antigens, Phialophora antigens, Prototheca antigens, Pseudallescheria antigens, Pseudomicrodochium antigens, Pythium antigens, Rhinosporidium antigens, Rhizopus antigens, Scolecobasidium antigens, Sporothrix antigens, Stemphylium antigens, Trichophyton antigens, Trichosporon antigens, and Xylohypha antigens.

Examples of protozoan parasite antigens include, but are not limited to, Babesia antigens, Balantidium antigens, Besnoitia antigens, Cryptosporidium antigens, Eimeri antigens a antigens, Encephalitozoon antigens, Entamoeba antigens, Giardia antigens, Hammondia antigens, Hepatozoon antigens, Isospora antigens, Leishmania antigens, Microsporidia antigens, Neospora antigens, Nosema antigens, Pentatrichomonas antigens, Plasmodium antigens, e.g., P. falciparum circumsporozoite (PfCSP), sporozoite surface protein 2 (PfSSP2), carboxyl terminus of liver state antigen 1 (PfLSA-1 c-term), and exported protein 1 (PfExp-1) antigens, Pneumocystis antigens, Sarcocystis antigens, Schistosoma antigens, Theileria antigens, Toxoplasma antigens, and Trypanosoma antigens.

Examples of helminth parasite antigens include, but are not limited to, Acanthocheilonema antigens, Aelurostrongylus antigens, Ancylostoma antigens, Angiostrongylus antigens, Ascaris antigens, Brugia antigens, Bunostomum antigens, Capillaria antigens, Chabertia antigens, Cooperia antigens, Crenosoma antigens, Dictyocaulus antigens, Dioctophyme antigens, Dipetalonema antigens, Diphyllobothrium antigens, Diplydium antigens, Dirofilaria antigens, Dracunculus antigens, Enterobius antigens, Filaroides,antigens Haemonchus antigens, Lagochilascaris antigens, Loa antigens, Mansonella antigens, Muellerius antigens, Nanophyetus antigens, Necator antigens, Nematodirus antigens, Oesophagostomum antigens, Onchocerca antigens, Opisthorchis antigens, Ostertagia antigens, Parafilaria antigens, Paragonimus antigens, Parascaris antigens, Physaloptera antigens, Protostrongylus antigens, Setaria antigens, Spirocerca,antigens Spirometra antigens, Stephanofilaria antigens, Strongyloides antigens, Strongylus antigens, Thelazia antigens, Toxascaris antigens, Toxocara antigens, Trichinella antigens, Trichostrongylus antigens, Trichuris antigens. Uncinaria antigens, and Wuchereria antigens.

In certain selection and screening schemes in which immunoglobulin molecules are expressed intracellularly, the host cells are “contacted” with antibody specific for a cell surface antigen by a method which will allow the antibody to bind, thereby allowing the host cells which specifically bind the antibody to be distinguished from those host cells which do not bind the antibody, or vice versa when cells which have reduced expression of the cell surface antigen are desired. Any method which allows host cells expressing a cell surface antigen to interact with the antibody is included. For example, if the host cells are in suspension, and the antibody is attached to a solid substrate, cells which specifically bind to the antibody will be trapped on the solid substrate, allowing those cells which do not bind the antigen to be washed away, and the bound cells to be subsequently recovered, or vice versa when cells which have lost expression of the cell surface antigen are desired. Alternatively, if the host cells are attached to a solid substrate, and by specifically binding antibody cells are caused to be released from the substrate (e.g., by cell death), they can be recovered from the cell supernatant. Preferred methods by which to allow host cells of the invention to contact antibody, especially using libraries constructed in vaccinia virus vectors by trimolecular recombination, are disclosed herein.

Recovery. After host cells which exhibit the desired, predetermined modified phenotype or which express a two-hybrid gene construct have been recovered, polynucleotides of the library are recovered from those host cells. By “recovery” is meant a crude separation of a desired component from those components which are not desired. For example, host cells are “recovered” based on their detachment from a solid substrate, and polynucleotides of the library are recovered from those cells by crude separation from other cellular components. It is to be noted that the term “recovery” does not imply any sort of purification or isolation away from viral and other components. Recovery of polynucleotides may be accomplished by any standard method known to those of ordinary skill in the art. In a preferred aspect, the polynucleotides are recovered by harvesting infectious virus particles, for example, particles of a vaccinia virus vector into which the library has been constructed, which were contained in those host cells exhibit a desired, predetermined modified phenotype or which express a two-hybrid gene construct.

In certain embodiments, after the identification of pools containing cells exhibiting a desired, predetermined modified phenotype, further screening steps are carried out until host cells which produce the desired intracellular immunoglobulin molecules are recovered, and then polynucleotides of the library are recovered from those host cells.

As will be readily appreciated by those of ordinary skill in the art, identification of polynucleotides encoding intracellular immunoglobulin molecules, or fragments thereof, may require two or more rounds of selection as described herein, and will necessarily require two or more rounds of screening as described herein. A single round of selection may not necessarily result in isolation of a pure set of polynucleotides encoding the desired first intracellular immunoglobulin subunit polypeptides; the mixture obtained after a first round may be enriched for the desired polynucleotides but may also be contaminated with non-target insert sequences. Screening assays described herein identify pools containing the positive host cells (e.g., those exhibiting a desired, predetermined modified phenotype), but such pools will also contain non-positive host cells. Therefore, the positive pools are further fractionated and subjected to further rounds of screening. Thus, identification of polynucleotides encoding an intracellular immunoglobulin subunit polypeptide which, in association with a second intracellular immunoglobulin subunit polypeptide, is capable of forming a desired intracellular immunoglobulin molecule, or fragment thereof, may require or benefit from several rounds of selection and/or screening, which thus increases the proportion of cells containing the desired polynucleotides. Accordingly, this embodiment further provides that the polynucleotides recovered after the first round be introduced into a second population of cells and be subjected to a second round of selection.

Accordingly, for intracellular immunoglobulin molecules, or fragments thereof, the first selection step, as described, may, or must be repeated one or more times, thereby enriching for the polynucleotides encoding the desired intracellular immunoglobulin molecules or fragments. In order to repeat the first step of this embodiment, those polynucleotides, or pools of polynucleotides, recovered as described above are introduced into a population of host cells capable of expressing the intracellular immunoglobulin molecules, or fragments thereof, encoded by the polynucleotides in the library. The host cells may be of the same type used in the first round of selection, or may be a different host cell, as long as they are capable of expressing intracellular immunoglobulin molecules, and are capable of exhibiting the desired predetermined modified phenotype. The second library of polynucleotides are also introduced into these host cells, and expression of intracellular immunoglobulin molecules, or fragments thereof, is permitted. The cells are similarly subjected to screening or selection, and polynucleotides of the library are again recovered from those cells or pools of host cells which exhibit a modified phenotype. These steps may be repeated one or more times, resulting in enrichment for polynucleotides derived from the library which encode an intracellular immunoglobulin subunit polypeptide which, as part of an intracellular immunoglobulin molecule, or an intracellular immunoglobulin fragment, directly or indirectly induces a desired, predetermined modified phenotype in a eukaryotic host cell.

Following suitable enrichment for the desired polynucleotides from the library as described above, those polynucleotides which have been recovered are “isolated,” i.e., they are substantially removed from their native environment and are largely separated from polynucleotides in the library which do not encode the intracellullar immunoglobulin molecules or fragments of interest. For example, cloned polynucleotides contained in a vector are considered isolated for the purposes of the present invention. It is understood that two or more different intracellular immunoglobulin molecules, or fragments thereof, which similarly modify the same phenotype or induce expression of a reporter gene can be recovered by the methods described herein. Accordingly, a mixture of such polynucleotides also considered to be “isolated.” Further examples of isolated polynucleotides include those maintained in heterologous host cells or purified (partially or substantially) DNA molecules in solution. However, a polynucleotide contained in a clone that is a member of a mixed library and that has not been isolated from other clones of the library is not “isolated” for the purposes of this invention. For example, a polynucleotide contained in a virus vector is “isolated” after it has been recovered, and plaque purified, and a polynucleotide contained in a plasmid vector is isolated after it has been expanded from a single bacterial colony.

Given that an antigen may comprise two or more epitopes, and several different immunoglobulin molecules may bind to any given epitope, it is contemplated that several suitable polynucleotides, e.g., two, three, four, five, ten, 100 or more polynucleotides, may be recovered from the first step of this embodiment, all of which may encode an intracellular immunoglobulin subunit polypeptide which, when combined with a suitable intracellular immunoglobulin subunit polypeptide encoded by a polynucleotide of the second library, will form an intracellular immunoglobulin molecule or fragment thereof, capable of directly or indirectly inducing a desired, predetermined, modified phenotype. It is contemplated that each different polynucleotide recovered from the first library would be separately isolated. However, these polynucleotides may be isolated as a group of polynucleotides which encode polypeptides with the same antigen specificity, and these polynucleotides may be “isolated” together. Such mixtures of polynucleotides, whether separately isolated or collectively isolated, may be introduced into host cells in the second step, as explained below, either individually, or with two, three, four, five, ten, 100 or more of the polynucleotides pooled together.

Once one or more suitable polynucleotides from the first library are isolated, in the second step, one or more polynucleotides are identified in the second library which encode intracellular immunoglobulin subunit polypeptide(s) capable of associating with the intracellular immunoglobulin subunit polypeptide(s) encoded by the polynucleotides isolated from the first library to form an intracellular immunoglobulin or fragment thereof which directly or indirectly induces a desired, predetermined modified phenotype.

Accordingly, the second step comprises introducing into a population of host cells, capable of expressing an immunoglobulin molecule, the second library of polynucleotides encoding a second intracellular immunoglobulin subunit polypeptide, introducing into the same population of host cells at least one of the polynucleotides isolated from the first library as described above, permitting expression of intracellular immunoglobulin molecules, or fragments thereof, screening or selecting host cells exhibiting a modified phenotype, and recovering polynucleotides of the second library from those host cells which exhibit a modified phenotype. The second step is thus carried out very similarly to the first step, except that the second intracellular immunoglobulin subunit polypeptides encoded by the polynucleotides of the second library are combined in the host cells with just those polynucleotides isolated from the first library. As mentioned above, a single cloned polynucleotide isolated from the first library may be used, or alternatively a pool of several polynucleotides isolated from the first library may be introduced simultaneously.

As with the first step described above, one or more rounds of enrichment are carried out, i.e., either selection or screening of successively smaller pools, thereby enriching for polynucleotides of the second library which encode a second intracellular immunoglobulin subunit polypeptide which, as part of an intracellular immunoglobulin or fragment thereof, induces a desired, predetermined modified phenotype. Also as with the first step, one or more desired polynucleotides from the second library are then isolated. If a pool of isolated polynucleotides is used in the earlier rounds of enrichment during the second step, preferred subsequent enrichment steps may utilize smaller pools of polynucleotides isolated from the first library, or even more preferably individual cloned polynucleotides isolated from the first library. For any individual polynucleotide isolated from the first library which is then used in the selection process for polynucleotides of the second library, it is possible that several, i.e. two, three, four, five, ten, 100, or more polynucleotides may be isolated from the second library which encode a second intracellular immunoglobulin subunit polypeptide capable of associating with a first intracellular immunoglobulin subunit polypeptide encoded by a polynucleotide isolated from the first library to form an intracellular immunoglobulin molecule, or fragment thereof, which directly or indirectly induces a desired, predetermined modified phenotype.

In contrast to bivalent intracellular immunoglobulin molecules, or fragments thereof, the selection/screening methods for libraries encoding intracellular single-chain immunoglobulins require only one library rather than first and second libraries, and only one selection/screening step is necessary. Similar to each of the two-steps for the intracellular immunoglobulin molecules, or fragments thereof, this one-step selection/screening method may also benefit from two or more rounds of enrichment.

Vectors. In constructing antibody libraries in eukaryotic cells, any standard vector which allows expression in eukaryotic cells may be used. For example, the library could be constructed in a virus, plasmid, phage, or phagemid vector as long as the particular vector chosen comprises transcription and translation regulatory regions capable of functioning in eukaryotic cells.

However, antibody libraries as described above are preferably constructed in eukaryotic virus vectors.

Eukaryotic virus vectors may be of any type, e.g., animal virus vectors or plant virus vectors. The naturally-occurring genome of the virus vector may be RNA, either positive strand, negative strand, or double stranded, or DNA, and the naturally-occurring genomes may be either circular or linear. Of the animal virus vectors, those that infect either invertebrates, e.g., insects, protozoans, or helminth parasites; or vertebrates, e.g., mammals, birds, fish, reptiles, and amphibians are included. The choice of virus vector is limited only by the maximum insert size, and the level of protein expression achieved. Suitable virus vectors are those that infect yeast and other fungal cells, insect cells, protozoan cells, plant cells, bird cells, fish cells, reptilian cells, amphibian cells, or mammalian cells, with mammalian virus vectors being particularly preferred. Any standard virus vector could be used in the present invention, including, but not limited to poxvirus vectors (e.g., vaccinia virus), herpesvirus vectors (e.g., herpes simplex virus), adenovirus vectors, baculovirus vectors, retrovirus vectors, picoma virus vectors (e.g., poliovirus), alphavirus vectors (e.g., sindbis virus), and enterovirus vectors (e.g., mengovirus). DNA virus vectors, e.g., poxvirus, herpes virus, baculovirus, and adenovirus are preferred. As described in more detail below, the poxviruses, particularly orthopoxviruses, and especially vaccinia virus, are particularly preferred. In a preferred embodiment, host cells are utilized which are permissive for the production of infectious viral particles of whichever virus vector is chosen. Many standard virus vectors, such as vaccinia virus, have a very broad host range, thereby allowing the use of a large variety of host cells.

As mentioned herein, the first and second libraries of the invention may be constructed in the same vector, or may be constructed in different vectors. However, in preferred embodiments, the first and second libraries are prepared such that polynucleotides of the first library can be conveniently recovered, e.g., separated, from the polynucleotides of the second library in the first step, and the polynucleotides of the second library can be conveniently recovered from the polynucleotides of the first library in the second step. For example, in the first step, if the first library is constructed in a virus vector, and the second library is constructed in a plasmid vector, the polynucleotides of the first library are easily recovered as infectious virus particles, while the polynucleotides of the second library are left behind with cellular debris. Similarly, in the second step, if the second library is constructed in a virus vector, while the polynucleotides of the first library isolated in the first step are introduced in a plasmid vector, infectious virus particles containing polynucleotides of the second library are easily recovered.

When the second library of polynucleotides, or the polynucleotides isolated from the first library are introduced into host cells in a plasmid vector, it is preferred that the intracellular immunoglobulin subunit polypeptides encoded by polynucleotides comprised in such plasmid vectors be operably associated with transcriptional regulatory regions which are driven by proteins encoded by virus vector which contains the other library. For example, if the first library is constructed in a poxvirus vector, and the second library is constructed in a plasmid vector, it is preferred that the polynucleotides encoding the second intracellular immunoglobulin subunit polypeptides constructed in the plasmid library be operably associated with a transcriptional control region, preferably a promoter, which functions in the cytoplasm of poxvirus-infected cells. Similarly in the second step, if it is desired to insert the polynucleotides isolated from the first library into a plasmid vector, and the second library is constructed in a poxvirus vector, it is preferred that polynucleotides isolated from the first library and inserted into plasmids be operably associated with a transcriptional regulatory region, preferably a promoter, which functions in the cytoplasm of poxvirus-infected cells. Suitable and preferred examples of such transcriptional control regions are disclosed herein. In this way, the polynucleotides of the second library are only expressed in those cells which have also been infected by a poxvirus.

However, it is convenient to be able to maintain both the first and second libraries, as well as those polynucleotides isolated from the first library, in just a virus vector rather than having to maintain one or both of the libraries in two different vector systems. Accordingly, the present invention provides that samples of the first or second libraries, maintained in a virus vector, are inactivated such that the virus vector infects cells and the genome of virus vector is transcribed, but the vector is not replicated, i.e., when the virus vector is introduced into cells, gene products carried on the virus genome, e.g., intracellular immunoglobulin subunit polypeptides, are expressed, but infectious virus particles are not produced.

The single-chain fragment library is preferably constructed in a poxvirus vector, preferably vaccinia virus.

The ability to synthesize and assemble intracellular immunoglobulin molecules, or fragments thereof, in eukaryotic cells from one (i.e., single-chain fragments) or two libraries of polynucleotides encoding intracellular immunoglobulin subunit polypeptides provides a significant improvement over the methods of producing single-chain antibodies in bacterial systems, in that the two-step selection process can be the basis for selection or screening of intracellular immunoglobulin molecules, or fragments thereof, with a variety of specificities and/or which induce a variety of phenotypic modifications. Additionally, these methods in eukaryotic cells allow one obtain intracellular immunoglobulin molecules, or fragments thereof, that interfere with a eukaryotic, especially a higher eukaryotic, gene product with efficiency.

Examples of specific embodiments which further illustrate, but do not limit this embodiment, are provided in the Examples below. As described in detail herein, selection of specific intracellular immunoglobulin subunit polypeptides, e.g., immunoglobulin heavy and light chains, is accomplished in two phases. First, a library of diverse heavy chains from immunoglobulin producing cells of either naïve or immunized donors is constructed in a eukaryotic virus vector, for example, a poxvirus vector, and a similarly diverse library of immunoglobulin light chains is constructed either in a plasmid vector, in which expression of the recombinant gene is regulated by a virus promoter, or in a eukaryotic virus vector which has been inactivated, e.g., through psoralen and UV treatment. Host cells capable of expressing intracellular immunoglobulin molecules, or antigen-specific fragments thereof, are infected with virus vector encoding the heavy chain library at a multiplicity of infection of about 1 (MOI=1). “Multiplicity of infection” refers to the average number of virus particles available to infect each host cell. For example, if an MOI of 1, i.e., an infection where, on average, each cell is infected by one virus particle, is desired, the number of infectious virus particles to be used in the infection is adjusted to be equal to the number of cells to be infected.

According to this strategy, host cells are either transfected with the light chain plasmid library, or infected with the inactivated light chain virus library under conditions which allow, on average, 10 or more separate polynucleotides encoding light chain polypeptides to be taken up and expressed in each cell. Under these conditions, a single host cell can express multiple intracellular immunoglobulin molecules, or fragments thereof, with different light chains associated with the same heavy chains in characteristic H₂L₂ structures or fragments thereof in each host cell.

It will be appreciated by those of ordinary skill in the art that controlling the number of plasmids taken up by a cell is difficult, because successful transfection depends on inducing a competent state in cells which may not be uniform and could lead to taking up variable amounts of DNA. Accordingly, in those embodiments where it is desired to carefully control the number of polynucleotides from the second library which are introduced into each infected host cell, the use of an inactivated virus vector is preferred, because the multiplicity of infection of viruses is more easily controlled.

The expression of multiple light chains in a single host cell, associated with a single heavy chain, has the effect of reducing the overall avidity of antigen-immunoglobulin interactions, but may be beneficial for selection of relatively high affinity binding sites. As used herein, the term “affinity” refers to a measure of the strength of the binding of an individual epitope with the CDR of an immunoglobulin molecule. See, e.g., Harlow at pages 27-28. As used herein, the term “avidity” refers to the overall stability of the complex between a population of immunoglobulins and an antigen, that is, the functional combining strength of an immunoglobulin mixture with the antigen. See, e.g., Harlow at pages 29-34. Avidity is related to both the affinity of individual immunoglobulin molecules in the population with specific epitopes, and also the valencies of the immunoglobulins and the antigen. For example, the interaction between a bivalent monoclonal antibody and an antigen with a highly repeating epitope structure, such as a polymer, would be one of high avidity. As will be appreciated by those of ordinary skill in the art, if a host cell expresses immunoglobulin molecules on its surface, each comprising a given heavy chain, but where different immunoglobulin molecules on the surface or intracellularly comprise different light chains, the “avidity” of that host cell for a given antigen will be reduced. However, the possibility of recovering a group of immunoglobulin molecules which are related in that they comprise a common heavy chain, but which, through association with different light chains, react with a particular antigen with a spectrum of affinities, is increased. Accordingly, by adjusting the number of different light chains, or fragments thereof, which are allowed to associate with a certain number of heavy chains, or fragments thereof in a given host cell, the present invention provides a method to select for and enrich for intracellular immunoglobulin molecules, or antigen-specific fragments thereof, with varied affinity levels.

In utilizing this strategy in the first step of the method for selecting intracellular immunoglobulin molecules, or antigen-specific fragments thereof as described above, the first library is preferably constructed in a eukaryotic virus vector, and the host cells are infected with the first library at an MOI ranging from about 1 to about 10, preferably about 1, while the second library is introduced under conditions which allow up to 20 polynucleotides of said second library to be taken up by each infected host cell. For example, if the second library is constructed in an inactivated virus vector, the host cells are infected with the second library at an MOI ranging from about 1 to about 20, although MOIs higher or lower than this range may be desirable depending on the virus vector used and the characteristics of the intracellular immunoglobulin molecules desired. If the second library is constructed in a plasmid vector, transfection conditions are adjusted to allow anywhere from 0 plasmids to about 20 plasmids to enter each host cell. Selection for lower or higher affinity responses to antigen is controlled by increasing or decreasing the average number of polynucleotides of the second library allowed to enter each infected cell.

More preferably, where the first library is constructed in a virus vector, host cells are infected with the first library at an MOI ranging from about 1-9, about 1-8, about 1-7, about 1-6, about 1-5, about 1-4, or about 1-2. In other words, host cells are infected with the first library at an MOI of about 10, about 9, about 8, about 7, about 6, about 5, about 4, about 3, about 2, or about 1. Most preferably, host cells are infected with the first library at an MOI of about 1.

Where the second library is constructed in a plasmid vector, the plasmid vector is more preferably introduced into host cells under conditions which allow up to about 19, about 18, about 17, about 16, about 15, about 14, about 13, about 12, about 10, about 9, about 8, about 7, about 6, about 5, about 4, about 3 about 2, or about 1 polynucleotide(s) of the second library to be taken up by each infected host cell. Most preferably, where the second library is constructed in a plasmid vector, the plasmid vector is introduced into host cells under conditions which allow up to about 10 polynucleotides of the second library to be taken up by each infected host cell.

Similarly, where the second library is constructed in an inactivated virus vector, it is more preferred to introduce the second library into host cells at an MOI ranging from about 1-19, about 2-18, about 3-17, about 4-16, about 5-15, about 6-14, about 7 -13, about 8-12, or about 9-11. In other words, host cells are infected with the second library at an MOI of about 20, about 19, about 18, about 17, about 16, about 15, about 14, about 13, about 12, about 11, about 10, about 9, about 8, about 7, about 6, about 5, about 4, about 3about 2, or about 1. In a most preferred aspect, host cells are infected with the second library at an MOI of about 10. As will be understood by those of ordinary skill in the art, the titer, and thus the “MOI” of an inactivated virus cannot be directly measured, however, the titer may be inferred from the titer of the starting infectious virus stock which was subsequently inactivated.

In a most preferred aspect, the first library is constructed in a virus vector and the second library is constructed in a virus vector which has been inactivated, the host cells are infected with said first library at an MOI of about 1, and the host cells are infected with the second library at an MOI of about 10.

In the present invention, a preferred virus vector is derived from a poxvirus, e.g., vaccinia virus. If the first library encoding the first intracellular immunoglobulin subunit polypeptide is constructed in a poxvirus vector and the expression of second intracellular immunoglobulin subunit polypeptides, encoded by the second library constructed either in a plasmid vector or an inactivated virus vector, are regulated by a poxvirus promoter, high levels of the second intracellular immunoglobulin subunit polypeptide are expressed in the cytoplasm of the poxvirus infected cells without a requirement for nuclear integration.

In the second step of the intracellular immunoglobulin selection as described above, the second library is preferably constructed in an infectious eukaryotic virus vector, and the host cells are infected with the second library at an MOI ranging from about 1 to about 10. More preferably, where the second library is constructed in a virus vector, host cells are infected with the second library at an MOI ranging from about 1-9, about 1-8, about 1-7, about 1-6, about 1-5, about 1-4, or about 1-2. In other words, host cells are infected with the second library at an MOI of about 10, about 9, about 8, about 7, about 6, about 5, about 4, about 3, about 2, or about 1. Most preferably, host cells are infected with the second library at an MOI of about 1.

In the second step of the intracellular immunoglobulin selection, polynucleotides from the first library have been isolated. In certain embodiments, a single first library polynucleotide, i.e., a clone, is introduced into the host cells used to isolate polynucleotides from the second library. In this situation, the polynucleotides isolated from the first library are introduced into host cells under conditions which allow at least about 1 polynucleotide per host cell. However, since all the polynucleotides being introduced from the first library will be the same, i.e., copies of a cloned polynucleotide, the number of polynucleotides introduced into any given host cell is less important. For example, if a cloned polynucleotide isolated from the first library is contained in an inactivated virus vector, that vector would be introduced at an MOI of about 1, but an MOI greater than 1 would be acceptable. Similarly, if a cloned polynucleotide isolated from the first library is introduced in a plasmid vector, the number of plasmids which are introduced into any given host cell is of little importance, rather, transfection conditions should be adjusted to insure that at least one polynucleotide is introduced into each host cell. An alternative embodiment may be utilized if, for example, several different polynucleotides were isolated from the first library. In this embodiment, pools of two or more different polynucleotides isolated from the first library may be advantageously introduced into host cells infected with the second library of polynucleotides. In this situation, if the polynucleotides isolated from the first library are contained in an inactivated virus vector, an MOI of inactivated virus particles of greater than about 1, e.g., about 2, about 3, about 4, about 5, or more may be preferred, of if the polynucleotides isolated from the first library are contained in a plasmid vector, conditions which allow at least about 2, 3, 4, 5, or more polynucleotides to enter each cell, may be preferred.

Poxvirus Vectors. As noted above, a preferred virus vector for use in the present invention is a poxvirus vector. “Poxvirus” includes any member of the family Poxviridae, including the subfamililes Chordopoxviridae (vertebrate poxviruses) and Entomopoxviridae (insect poxviruses). See, for example, B. Moss in: Virology, 2d Edition, B. N. Fields, D. M. Knipe et al., Eds., Raven Press, p. 2080 (1990). The chordopoxviruses comprise, inter alia, the following genera: Orthopoxvirus (e.g., vaccinia, variola virus, raccoon poxvirus); Avipoxvirus (e.g., fowlpox); Capripoxvirus (e.g, sheeppox) Leporipoxvirus (e.g., rabbit (Shope) fibroma, and myxoma); and Suipoxvirus (e.g., swinepox). The entomopoxviruses comprise three genera: A, B and C. In the present invention, orthopoxviruses are preferred. Vaccinia virus is the prototype orthopoxvirus, and has been developed and is well-characterized as a vector for the expression of heterologous proteins. In the present invention, vaccinia virus vectors, particularly those that have been developed to perform trimolecular recombination, are preferred. However, other orthopoxviruses, in particular, raccoon poxvirus have also been developed as vectors and in some applications, have superior qualities.

Poxviruses are distinguished by their large size and complexity, and contain similarly large and complex genomes. Notably, poxviruses replication takes place entirely within the cytoplasm of a host cell. The central portions of poxvirus genomes are similar, while the terminal portions of the virus genomes are characterized by more variability. Accordingly, it is thought that the central portion of poxvirus genomes carry genes responsible for essential functions common to all poxviruses, such as replication. By contrast, the terminal portions of poxvirus genomes appear responsible for characteristics such as pathogenicity and host range, which vary among the different poxviruses, and may be more likely to be non-essential for virus replication in tissue culture. It follows that if a poxvirus genome is to be modified by the rearrangement or removal of DNA fragments or the introduction of exogenous DNA fragments, the portion of the naturally-occurring DNA which is rearranged, removed, or disrupted by the introduction of exogenous DNA is preferably in the more distal regions though to be non-essential for replication of the virus and production if infectious virions in tissue culture.

The naturally-occurring vaccinia virus genome is a cross-linked, double stranded linear DNA molecule, of about 186,000 base pairs (bp), which is characterized by inverted terminal repeats. The genome of vaccinia virus has been completely sequenced, but the functions of most gene products remain unknown.

Goebel, S. J., et al., Virology 179:247-266,517-563 (1990); Johnson, G. P., et al., Virology 196:381-401. A variety of non-essential regions have been identified in the vaccinia virus genome. See, e.g., Perkus, M. E., et al., Virology 152:285-97 (1986); and Kotwal, G. J. and Moss B., Virology 167:524-37.

In those embodiments where poxvirus vectors, in particular vaccinia virus vectors, are used to express immunglobulin subunit polypeptides or single-chain fragments, any suitable poxvirus vector may be used. It is preferred that the libraries of intracellular immunoglobulin subunit polypeptides or single-chain fragments be carried in a region of the vector which is non-essential for growth and replication of the vector so that infectious viruses are produced. Although a variety of non-essential regions of the vaccinia virus genome have been characterized, the most widely used locus for insertion of foreign genes is the thymidine kinase locus, located in the HindIII J fragment in the genome. In certain preferred vaccinia virus vectors, the tk locus has been engineered to contain one or two unique restriction enzyme sites, allowing for convenient use of the trimolecular recombination method of library generation. See herein, and also Zauderer, PCT Publication No. WO 00/028016.

Libraries of polynucleotides encoding intracellular immunoglobulin subunit polypeptides or single-chain fragments are inserted into poxvirus vectors, particularly vaccinia virus vectors, under operable association with a transcriptional control region which functions in the cytoplasm of a poxvirus-infected cell.

Poxvirus transcriptional control regions comprise a promoter and a transcription termination signal. Gene expression in poxviruses is temporally regulated, and promoters for early, intermediate, and late genes possess varying structures. Certain poxvirus genes are expressed constitutively, and promoters for these “early-late” genes bear hybrid structures. Synthetic early-late promoters have also been developed. See Hammond J. M., et al., J. Virol. Methods 66:135-8 (1997); Chakrabarti S., et al., Biotechniques 23:1094-7 (1997). In the present invention, any poxvirus promoter may be used, but use of early, late, or constitutive promoters may be desirable based on the host cell and/or selection scheme chosen. Typically, the use of constitutive promoters is preferred.

Examples of early promoters include the 7.5-kD promoter (also a late promoter), the DNA pol promoter, the tk promoter, the RNA pol promoter, the 19-kD promoter, the 22-kD promoter, the 42-kD promoter, the 37-kD promoter, the 87-kD promoter, the H3′ promoter, the H6 promoter, the D1 promoter, the D4 promoter, the D5 promoter, the D9 promoter, the D12 promoter, the I3 promoter, the M1 promoter, and the N2 promoter. See, e.g., Moss, B., “Poxviridae and their Replication” IN Virology, 2d Edition, B. N. Fields, D. M. Knipe et al., Eds., Raven Press, p.2088 (1990). Early genes transcribed in vaccinia virus and other poxviruses recognize the transcription termination signal TTTTTNT, where N can be any nucleotide. Transcription normally terminates approximately 50 bp upstream of this signal. Accordingly, if heterologous genes are to be expressed from poxvirus early promoters, care must be taken to eliminate occurrences of this signal in the coding regions for those genes. See, e.g., Earl, P. L., et al., J. Virol. 64:2448-51 (1990).

Example of late promoters include the 7.5-kD promoter, the MIL promoter, the 37-kD promoter, the 11-kD promotor, the 11L promoter, the 12L promoter, the 13L promoter, the 15L promoter, the 17L promoter, the 28-kD promoter, the H1L promoter, the H3L promoter, the H5L promoter, the H6L promoter, the H8L promoter, the D11L promoter, the D12L promotor, the D13L promoter, the A1L promoter, the A2L promoter, the A3L promoter, and the P4b promoter. See, e.g., Moss, B., “Poxviridae and their Replication” IN Virology, 2d Edition, B. N. Fields, D. M. Knipe et al., Eds., Raven Press, p. 2090 (1990). The late promoters apparently do not recognize the transcription termination signal recognized by early promoters.

Preferred constitutive promoters for use in the present invention include the synthetic early-late promoters described by Hammond and Chakrabarti, the MH-5 early-late promoter, and the 7.5-kD or “p7.5” promoter. Examples utilizing these promoters are disclosed herein.

Attenuated and Defective Viral Vectors. As will be discussed in more detail below, certain selection and screening methods based on host cell death require that the mechanisms leading to cell death occur prior to any cytopathic effect (CPE) caused by virus infection. The kinetics of the onset of CPE in virus-infected cells is dependent on the virus used, the multiplicity of infection, and the type of host cell. For example, in many tissue culture lines infected with vaccinia virus at an MOI of about 1, CPE is not significant until well after 48 to 72 hours post-infection. This allows a 2 to 3 day time frame for high level expression of intracellular immunoglobulin molecules, and screening or selection independent of CPE caused by the vector. However, this time frame may not be sufficient for certain selection methods, especially where higher MOIs are used, and further, the time before the onset of CPE may be shorter in a desired cell line. There is, therefore, a need for virus vectors, particularly poxvirus vectors such as vaccinia virus, with attenuated cytopathic effects so that, wherever necessary, the time frame of selection can be extended.

For example, certain attenuations are achieved through genetic mutation.

These may be fully defective mutants, i.e., the production of infectious virus particles requires helper virus, or they may be conditional mutants, e.g., temperature sensitive mutants. Conditional mutants are particularly preferred, in that the virus-infected host cells can be maintained in a non-permissive environment, e.g., at a non-permissive temperature, during the period where host gene expression is required, and then shifted to a permissive environment, e.g., a permissive temperature, to allow virus particles to be produced. Alternatively, a fully infectious virus may be “attenuated” by chemical inhibitors which reversibly block virus replication at defined points in the infection cycle. Chemical inhibitors include, but are not limited to hydroxyurea and 5-fluorodeoxyuridine. Virus-infected host cells are maintained in the chemical inhibitor during the period where host gene expression is required, and then the chemical inhibitor is removed to allow virus particles to be produced.

A number of attenuated poxviruses, in particular vaccinia viruses, have been developed. For example, modified vaccinia Ankara (MVA) is a highly attenuated strain of vaccinia virus that was derived during over 570 passages in primary chick embryo fibroblasts (Mayr, A. et al., Infection 3:6-14 (1975)). The recovered virus deleted approximately 15% of the wild type vaccinia DNA which profoundly affects the host range restriction of the virus. MVA cannot replicate or replicates very inefficiently in most mammalian cell lines. A unique feature of the host range restriction is that the block in non-permissive cells occurs at a relatively late stage of the replication cycle. Expression of viral late genes is relatively unimpaired but virion morphogenesis is interrupted (Suter, G. and Moss, B., Proc Natl Acad Sci USA 89:10847-51 (1992); Carroll, M. W. and Moss, B., Virology 238:198-211 (1997)). The high levels of viral protein synthesis even in non-permissive host cells make MVA an especially safe and efficient expression vector. However, because MVA cannot complete the infectious cycle in most mammalian cells, in order to recover infectious virus for multiple cycles of selection it will be necessary to complement the MVA deficiency by coinfection or superinfection with a helper virus that is itself deficient and that can be subsequently separated from infectious MVA recombinants by differential expansion at low MOI in MVA permissive host cells.

As an alternative to MVA, some strains of vaccinia virus that are deficient in an essential early gene have been shown to have greatly reduced inhibitory effects on host cell protein synthesis. Attenuated poxviruses which lack defined essential early genes have also been described. See, e.g., U.S. Pat. No. 5,766,882, by Falkner, et al. Examples of essential early genes which may be rendered defective include, but are not limited to the vaccinia virus 17L, F18R, D13L, D6R, A8L, J1R, E7L, F11L, E4L, I1L, J3R, J4R, H7R, and A6R genes. A preferred essential early gene to render defective is the D4R gene, which encodes a uracil DNA glycosylase enzyme. Vaccinia viruses defective in defined essential genes are easily propagated in complementing cell lines which provides the essential gene product.

As used herein, the term “complementation” refers to a restoration of a lost function in trans by another source, such as a host cell, transgenic animal or helper virus. The loss of function is caused by loss by the defective virus of the gene product responsible for the function. Thus, a defective poxvirus is a non-viable form of a parental poxvirus, and is a form that can become viable in the presence of complementation. The host cell, transgenic animal or helper virus contains the sequence encoding the lost gene product, or “complementation element.” The complementation element should be expressible and stably integrated in the host cell, transgenic animal or helper virus, and preferably would be subject to little or no risk for recombination with the genome of the defective poxvirus.

Viruses produced in the complementing cell line are capable of infecting non-complementing cells, and further are capable of high-level expression of early gene products. However, in the absence of the essential gene product, host shut-off, DNA replication, packaging, and production of infectious virus particles does not take place.

In particularly preferred embodiments described herein, selection of desired target gene products expressed in a complex library constructed in vaccinia virus is accomplished through coupling induction of expression of the complementation element to expression of the desired target gene product. Since the complementation element is only expressed in those host cells expressing the desired gene product, only those host cells will produce infectious virus which is easily recovered.

In a preferred aspect, inactivation of the library constructed in a eukaryotic virus vector is carried out by treating a sample of the library constructed in a virus vector with 4′-aminomethyl-trioxsalen (psoralen) and then exposing the virus vector to ultraviolet (UV) light. Psoralen and UV inactivation of viruses is well known to those of ordinary skill in the art. See, e.g., Tsung, K., et al., J. Virol. 70:165-171 (1996), which is incorporated herein by reference in its entirety.

Psoralen treatment typically comprises incubating a cell-free sample of the virus vector with a concentration of psoralen ranging from about 0.1 μg/ml to about 20 μg/ml, preferably about 1 μg/ml to about 17.5 μg/ml, about 2.5 μg/ml to about 15 μg/ml, about 5 μg/ml to about 12.5 μg/ml, about 7.5 μg/ml to about 12.5 μg/ml, or about 9 μg/ml to about 11 μg/ml. Accordingly, the concentration of psoralen may be about 0.1 μg/ml, 0.5 μg/ml,1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml. Preferably, the concentration of psoralen is about 10 μg/ml. As used herein, the term “about” takes into account that measurements of time, chemical concentration, temperature, pH, and other factors typically measured in a laboratory or production facility are never exact, and may vary by a given amount based on the type of measurement and the instrumentation used to make the measurement.

The incubation with psoralen is typically carried out for a period of time prior to UV exposure. This time period preferably ranges from about one minute to about 20 minutes prior to the UV exposure. Preferably, the time period ranges from about 2 minutes to about 19 minutes, from about 3 minutes to about 18 minutes, from about 4 minutes to about 17 minutes, from about 5 minutes to about 16 minutes, from about 6 minutes to about 15 minutes, from about 7 minutes to about 14 minutes, from about 8 minutes to about 13 minutes, or from about 9 minutes to about 12 minutes. Accordingly, the incubation time may be about 1 minute, about 2 minutes, about three minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. More preferably, the incubation is carried out for 10 minutes prior to the UV exposure.

The psoralen-treated viruses are then exposed to UV light. The UV may be of any wavelength, but is preferably long-wave UV light, e.g., about 365 nm. Exposure to UV is carried out for a time period ranging from about 0.1 minute to about 20 minutes. Preferably, the time period ranges from about 0.2 minute to about 19 minutes, from about 0.3 minute to about 18 minutes, from about 0.4 minute to about 17 minutes, from about 0.5 minute to about 16 minutes, from about 0.6 minute to about 15 minutes, from about 0.7 minute to about 14 minutes, from aboutO.8 minute to about 13 minutes, from about 0.9 minute to about 12 minutes from about 1 minute to about 11 minutes, from about 2 minutes to about 10 minutes, from about 2.5 minutes to about 9 minutes, from about 3 minutes to about 8 minutes, from about 4 minutes to about 7 minutes, or from about 4.5 minutes to about 6 minutes. Accordingly, the incubation time may be about 0.1 minute, about 0.5 minute, about 1 minute, about 2 minutes, about three minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. More preferably, the virus vector is exposed to UV light for a period of about 5 minutes.

The preferred embodiments relating to vaccinia virus may be modified in ways apparent to one of ordinary skill in the art for use with any poxvirus vector. In the direct selection method, vectors other than poxvirus or vaccinia virus may be used.

The Tri-Molecular Recombination Method. Traditionally, poxvirus vectors such as vaccinia virus have not been used to identify previously unknown genes of interest from a complex libraries because a high efficiency, high titer-producing method of constructing and screening libraries did not exist for vaccinia. The standard methods of heterologous protein expression in vaccinia virus involve in vivo homologous recombination and in vitro direct ligation. Using homologous recombination, the efficiency of recombinant virus production is in the range of approximately 0.1% or less. Although efficiency of recombinant virus production using direct ligation is higher, the resulting titer is relatively low. Thus, the use of vaccinia virus vector has been limited to the cloning of previously isolated DNA for the purposes of protein expression and vaccine development.

Tri-molecular recombination, as disclosed in Zauderer, PCT Publication No. WO 00/028016, is a novel, high efficiency, high titer-producing method for cloning in vaccinia virus. Using the tri-molecular recombination method, the present inventor has achieved generation of recombinant viruses at efficiencies of at least 90%, and titers at least at least 2 orders of magnitude higher than those obtained by direct ligation.

Thus, in a preferred embodiment, libraries of polynucleotides capable of expressing intracellular immunoglobulin subunit polypeptides or single-chain fragments are constructed in poxvirus vectors, preferably vaccinia virus vectors, by tri-molecular recombination.

By “tri-molecular recombination” or a “tri-molecular recombination method” is meant a method of producing a virus genome, preferably a poxvirus genome, and even more preferably a vaccinia virus genome comprising a heterologous insert DNA, by introducing two nonhomologous fragments of a virus genome and a transfer vector or transfer DNA containing insert DNA into a recipient cell, and allowing the three DNA molecules to recombine in vivo. As a result of the recombination, a viable virus genome molecule is produced which comprises each of the two genome fragments and the insert DNA.

Thus, the tri-molecular recombination method as applied to the present invention comprises: (a) cleaving an isolated virus genome, preferably a DNA virus genome, more preferably a linear DNA virus genome, and even more preferably a poxvirus or vaccinia virus genome, to produce a first viral fragment and a second viral fragment, where the first viral fragment is nonhomologous with the second viral fragment; (b) providing a population of transfer plasmids comprising polynucleotides which encode intracellular immunoglobulin subunit polypeptides, e.g., immunoglobulin light chains, immunoglobulin heavy chains, fragments of either, or single-chain fragments, through operable association with a transcription control region, flanked by a 5′ flanking region and a 3′ flanking region, wherein the 5′ flanking region is homologous to said the viral fragment described in (a), and the 3′ flanking region is homologous to said second viral fragment described in (a); and where the transfer plasmids are capable of homologous recombination with the first and second viral fragments such that a viable virus genome is formed; (c) introducing the transfer plasmids described in (b) and the first and second viral fragments described in (a) into a host cell under conditions where a transfer plasmid and the two viral fragments undergo in vivo homologous recombination, i.e., trimolecular recombination, thereby producing a viable modified virus genome comprising a polynucleotide which encodes an intracellular immunoglobulin subunit polypeptide; and (d) recovering modified virus genomes produced by this technique. Preferably, the recovered modified virus genome is packaged in an infectious viral particle.

By “recombination efficiency” or “efficiency of recombinant virus production” is meant the ratio of recombinant virus to total virus produced during the generation of virus libraries of the present invention. As shown in Example 5, the efficiency may be calculated by dividing the titer of recombinant virus by the titer of total virus and multiplying by 100%. For example, the titer is determined by plaque assay of crude virus stock on appropriate cells either with selection (e.g., for recombinant virus) or without selection (e.g., for recombinant virus plus wild type virus). Methods of selection, particularly if heterologous polynucleotides are inserted into the viral thymidine kinase (tk) locus, are well-known in the art and include resistance to bromdeoxyuridine (BDUR) or other nucleotide analogs due to disruption of the tk gene. Examples of selection methods are described herein.

By “high efficiency recombination” is meant a recombination efficiency of at least 1%, and more preferably a recombination efficiency of at least about 2%, 2.5%, 3%, 3.5%, 4%, 5%, 10%, 20%, 30%, 40%, 50%, 60%, 70%, 75%, 80%, 85%, 90%, 95%, or 99%.

A number of selection systems may be used, including but not limited to the thymidine kinase such as herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes which can be employed in tk⁻, hgprt⁻ or aprt⁻ cells, respectively. Also, antimetabolite resistance can be used as the basis of selection for the following genes: dhfr, which confers resistance to methotrexate (Wigler, et al., 1980, Natl. Acad. Sci. USA 77:3567; O'Hare,et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147).

Together, the first and second viral fragments or “arms” of the virus genome, as described above, preferably contain all the genes necessary for viral replication and for production of infectious viral particles. Examples of suitable arms and methods for their production using vaccinia virus vectors are disclosed herein. See also Falkner et al., U.S. Pat. No. 5,770,212 for guidance concerning essential regions for vaccinia replication.

However, naked poxvirus genomic DNAs such as vaccinia virus genomes cannot produce infectious progeny without virus-encoded protein protein(s)/function(s) associated with the incoming viral particle. The required virus-encoded functions, include an RNA polymerase that recognizes the transfected vaccinia DNA as a template, initiates transcription and, ultimately, replication of the transfected DNA. See Dorner, et al. U.S. Pat. No. 5,445,953.

Thus, to produce infectious progeny virus by trimolecular recombination using a poxvirus such as vaccinia virus, the recipient cell preferably contains packaging function. The packaging function may be provided by helper virus, i.e., a virus that, together with the transfected naked genomic DNA, provides appropriate proteins and factors necessary for replication and assembly of progeny virus.

The helper virus may be a closely related virus, for instance, a poxvirus of the same poxvirus subfamily as vaccinia, whether from the same or a different genus. In such a case it is advantageous to select a helper virus which provides an RNA polymerase that recognizes the transfected DNA as a template and thereby serves to initiate transcription and, ultimately, replication of the transfected DNA. If a closely related virus is used as a helper virus, it is advantageous that it be attenuated such that formation of infectious virus will be impaired. For example, a temperature sensitive helper virus may be used at the non-permissive temperature. Preferably, a heterologous helper virus is used. Examples include, but are not limited to a avipox virus such as fowlpox virus, or an ectromelia virus (mouse pox) virus. In particular, avipoxviruses are preferred, in that they provide the necessary helper functions, but do not replicate, or produce infectious virions in mammalian cells (Scheiflinger, et al., Proc. Natl. Acad. Sci. USA 89:9977-9981 (1992)). Use of heterologous viruses minimizes recombination events between the helper virus genome and the transfected genome which take place when homologous sequences of closely related viruses are present in one cell. See Fenner & Comben, Virology 5:530 (1958); Fenner, Virology 8:499 (1959).

Alternatively, the necessary helper functions in the recipient cell is supplied by a genetic element other than a helper virus. For example, a host cell can be transformed to produce the helper functions constitutively, or the host cell can be transiently transfected with a plasmid expressing the helper functions, infected with a retrovirus expressing the helper functions, or provided with any other expression vector suitable for expressing the required helper virus function. See Dorner, et al. U.S. Pat. No. 5,445,953.

According to the trimolecular recombination method, the first and second viral genomic fragments are unable to ligate or recombine with each other, i.e., they do not contain compatible cohesive ends or homologous regions, or alternatively, cohesive ends have been treated with a dephosphorylating enzyme. In a preferred embodiment, a virus genome comprises a first recognition site for a first restriction endonuclease and a second recognition site for a second restriction endonuclease, and the first and second viral fragments are produced by digesting the viral genome with the appropriate restriction endonucleases to produce the viral “arms,” and the first and second viral fragments are isolated by standard methods. Ideally, the first and second restriction endonuclease recognition sites are unique in the viral genome, or alternatively, cleavage with the two restriction endonucleases results in viral “arms” which include the genes for all essential functions, i.e., where the first and second recognition sites are physically arranged in the viral genome such that the region extending between the first and second viral fragments is not essential for virus infectivity.

In a preferred embodiment where a vaccinia virus vector is used in the trimolecular recombination method, a vaccinia virus vector comprising a virus genome with two unique restriction sites within the tk gene is used. In certain preferred vaccinia virus genomes, the first restriction enzyme is NotI, having the recognition site GCGGCCGC in the tk gene, and the second restriction enzyme is Apal, having the recognition site GGGCCC in the tk gene. Even more preferred are vaccinia virus vectors comprising a v7.5/tk virus genome or a vEL/tk virus genome.

According to this embodiment, a transfer plasmid with flanking regions capable of homologous recombination with the region of the vaccinia virus genome containing the thymidine kinase gene is used. A fragment of the vaccinia virus genome comprising the HindIII-J fragment, which contains the tk gene, is conveniently used.

Where the virus vector is a poxvirus, the insert polynucleotides are preferably operably associated with poxvirus expression control sequences, more preferably, strong constitutive poxvirus promoters such as p7.5 or a synthetic early/late promoter.

Accordingly, a transfer plasmid of the present invention comprises a polynucleotide encoding an intracellular immunoglobulin subunit polypeptide, e.g., an heavy chain, and immunoglobulin light chain, or an antigen-specific fragment of a heavy chain or a light chain, through operable association with a vaccinia virus p7.5 promoter, or a synthetic early/late promoter.

A preferred transfer plasmid of the present invention which comprises a polynucleotide encoding an immunoglobulin heavy chain polypeptide through operable association with a vaccinia virus p7.5 promoter is pVHE, which comprises the sequence:

GGCCAAAAATTGAAAAACTAGATCTATTTATTGCACGCGGCCGCAAACCA
TGGGATGGAGCTGTATCATCCTCTTCTTGGTAGCAACAGCTACAGGCGCG
CATATGGTCACCGTCTCCTCAGGGAGTGCATCCGCCCCAACCCTTTTCCC
CCTCGTCTCCTGTGAGAATTCCCCGTCGGATACGAGCAGCGTGGCCGTTG
GCTGCCTCGCACAGGACTTCCTTCCCGACTCCATCACTTTCTCCTGGAAA
TACAAGAACAACTCTGACATCAGCAGCACCCGGGGCTTCCCATCAGTCCT
GAGAGGGGGCAAGTACGCAGCCACCTCACAGGTGCTGCTGCCTTCCAAGG
ACGTCATGCAGGGCACAGACGAACACGTGGTGTGCAAAGTCCAGCACCCC
AACGGCAACAAAGAAAAGAACGTGCCTCTTCCAGTGATTGCTGAGCTGCC
TCCCAAAGTGAGCGTCTTCGTCCCACCCCGCGACGGCTTCTTCGGCAACC
CCCGCAGCAAGTCCAAGCTCATCTGCCAGGCCACGGGTTTCAGTCCCCGG
CAGATTCAGGTGTCCTGGCTGCGCGAGGGGAAGCAGGTGGGGTCTGGCGT
CACCACGGACCAGGTGCAGGCTGAGGCCAAAGAGTCTGGGCCCACGACCT
ACAAGGTGACTAGCACACTGACCATCAAAGAGAGCGACTGGCTCAGCCAG
AGCATGTTCACCTGCCGCGTGGATCACAGGGGCCTGACCTTCCAGCAGAA
TGCGTCCTCCATGTGTGTCCCCGATCAAGACACAGCCATCCGGGTCTTCG
CCATCCCCCCATCCTTTGCCAGCATCTTCCTCACCAAGTCCACCAAGTTG
ACCTGCCTGGTCACAGACCTGACCACCTATGACAGCGTGACCATCTCCTG
GACCCGCCAGAATGGCGAAGCTGTGAAAACCCACACCAACATCTCCGAGA
GCCACCCCAATGCCACTTTCAGCGCCGTGGGTGAGGCCAGCATCTGCGAG
GATGACTGGAATTCCGGGGAGAGGTTCACGTGCACCGTGACCCACACAGA
CCTGCCCTCGCCACTGAAGCAGACCATCTCCCGGCCCAAGGGGGTGGCCC
TGCACAGGCCCGATGTCTACTTGCTGCCACCAGCCCGGGAGCAGCTGAAC
CTGCGGGAGTCGGCCACCATCACGTGCCTGGTGACGGGCTTCTCTCCCGC
GGACGTCTTCGTGCAGTGGATGCAGAGGGGGCAGCCCTTGTCCCCGGAGA
AGTATGTGACCAGCGCCCCAATGCCTGAGCCCCAGGCCCCAGGCCGGTAC
TTCGCCCACAGCATCCTGACCGTGTCCGAAGAGGAATGGAACACGGGGGA
GACCTACACCTGCGTGGTGGCCCATGAGGCCCTGCCCAACAGGGTCACTG
AGAGGACCGTGGACAAGTCCACCGAGGGGGAGGTGAGCGCCGACGAGGAG
GGCTTTGAGAACCTGTGGGCCACCGCCTCCACCTTCATCGTCCTCTTCCT
CCTGAGCCTCTTCTACAGTACCACCGTCACCTTGTTCAAGGTGAAATGAG
TCGAC

designated herein as SEQ ID NO:74. PCR-amplified heavy chain variable regions may be inserted in-frame into unique BssHII (at nucleotides 96-100 of SEQ ID NO:74), and BstEII (nucleotides 106-112 of SEQ ID NO:74) sites, which are indicated above in bold.

Furthermore, pVHE may be used in those embodiments where it is desired to transfer polynucleotides isolated from the first library into a plasmid vector for subsequent selection of polynucleotides of the second library as described above.

Another preferred transfer plasmid of the present invention which comprises a polynucleotide encoding an immunoglobulin kappa light chain polypeptide through operable association with a vaccinia virus p7.5 promoter is pVKE, which comprises the sequence:

GGCCAAAAATTGAAAAACTAGATCTATTTATTGCACGCGGCCGCCCATGG
GATGGAGCTGTATCATCCTCTTCTTGGTAGCAACAGCTACAGGCGTGCAC
TTGACTCGAGATCAAACGAACTGTGGCTGCACCATCTGTCTTCATCTTCC
CGCCATCTGATGAGCAGTTGAAATCTGGAACTGCCTCTGTTGTGTGCCTG
CTGAATAACTTCTATCCCAGAGAGGCCAAAGTACAGTGGAAGGTGGATAA
CGCCCTCCAATCGGGTAACTCCCAGGAGAGTGTCACAGAGCAGGACAGCA
AGGACAGCACCTACAGCCTCAGCAGCACCCTGACGCTGAGCAAAGCAGAC
TACGAGAAACACAAAGTCTACGCCTGCGAAGTCACCCATCAGGGCCTGAG
CTCGCCCGTCACAAAGAGCTTCAACAGGGGAGAGTGTTAGGTCGAC

designated herein as SEQ ID NO:75. PCR-amplified kappa light chain variable regions may be inserted in-frame into unique ApaLI (nucleotides 95-100 of SEQ ID NO:75), and XhoI (nucleotides 105-110 of SEQ ID NO:75) sites, which are indicated above in bold.

Furthermore, pVKE may be used in those embodiments where it is desired to have polynucleotides of the second library in a a plasmid vector during the selection of polynucleotides of the first library as described above.

Another preferred transfer plasmid of the present invention which comprises a polynucleotide encoding an immunoglobulin lambda light chain polypeptide through operable association with a vaccinia virus p7.5 promoter is pVLE, which comprises the sequence:

GGCCAAAAATTGAAAAACTAGATCTATTTATTGCACGCGGCCGCCCATGG
GATGGAGCTGTATCATCCTCTTCTTGGTAGCAACAGCTACAGGCGTGCAC
TTGACTCGAGAAGCTTACCGTCCTACGAACTGTGGCTGCACCATCTGTCT
TCATCTTCCCGCCATCTGATGAGCAGTTGAAATCTGGAACTGCCTCTGTT
GTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGC
AGGACAGCAAGGACAGCACCTACAGCCTCAGCAGCACCCTGACGCTGAGC
AAAGCAGACTACGAGAAACACAAAGTCTACGCCTGCGAAGTCACCCATCA
GGGCCTGAGCTCGCCCGTCACAAAGAGCTTCAACAGGGGAGAGTGTTAGG
TCGAC

designated herein as SEQ ID NO:76. PCR-amplified lambda light chain variable regions may be inserted in-frame into unique ApaLI (nucleotides 95-100 of SEQ ID NO:76) and HindIII (nucleotides 111-116 of SEQ ID NO:76) sites, which are indicated above in bold.

Furthermore, pVLE may be used in those embodiments where it is desired to have polynucleotides of the second library in a a plasmid vector during the selection of polynucleotides of the first library as described above.

By “insert DNA” is meant one or more heterologous DNA segments to be expressed in the recombinant virus vector. According to the present invention, “insert DNAs” are polynucleotides which encode intracellular immunoglobulin subunit polypeptides. A DNA segment may be naturally occurring, non naturally occurring, synthetic, or a combination thereof. Methods of producing insert DNAs of the present invention are disclosed herein.

By “transfer plasmid” is meant a plasmid vector containing an insert DNA positioned between a 5′ flanking region and a 3′ flanking region as described above. The 5′ flanking region shares homology with the first viral fragment, and the 3′ flanking region shares homology with the second viral fragment. Preferably, the transfer plasmid contains a suitable promoter, such as a strong, constitutive vaccinia promoter where the virus vector is a poxvirus, upstream of the insert DNA. The term “vector” means a polynucleotide construct containing a heterologous polynucleotide segment, which is capable of effecting transfer of that polynucleotide segment into a suitable host cell. Preferably the polynucleotide contained in the vector is operably linked to a suitable control sequence capable of effecting the expression of the polynucleotide in a suitable host. Such control sequences include a promoter to effect transcription, an optional operator sequence to control such transcription, a sequence encoding suitable mRNA ribosome binding sites, and sequences which control the termination of transcription and translation. As used herein, a vector may be a plasmid, a phage particle, a virus, a messenger RNA, or simply a potential genomic insert. Once transformed into a suitable host, the vector may replicate and function independently of the host genome, or may in some instances, integrate into the genome itself. Typical plasmid expression vectors for mammalian cell culture expression, for example, are based on pRK5 (EP 307,247), pSV16B (WO 91/08291) and pVL1392 (Pharmingen).

However, “a transfer plasmid,” as used herein, is not limited to a specific plasmid or vector. Any DNA segment in circular or linear or other suitable form may act as a vehicle for transferring the DNA insert into a host cell along with the first and second viral “arms” in the tri-molecular recombination method. Other suitable vectors include lambda phage, mnRNA, DNA fragments, etc., as described herein or otherwise known in the art. A plurality of plasmids may be a “primary library” such as those described herein for lambda.

Modifications of Trimolecular Recombination. Trimolecular recombination can be used to construct cDNA libraries in vaccinia virus with titers of the order of about 10⁷ pfu. There are several factors that limit the complexity of these cDNA libraries or other libraries. These include: the size of the primary cDNA library or other library, such as a library of polynucleotides encoding intracellular immunoglobulin subunit polypeptides, that can be constructed in a plasmid vector, and the labor involved in the purification of large quantities (hundreds of micrograms) of virus “arms,” preferably vaccinia virus “arms” or other poxvirus “arms.” Modifications of trimolecular recombination that would allow for vaccinia or other virus DNA recombination with primary cDNA libraries or other libraries, such as polynucleotides encoding intracellular immunoglobulin subunit polypeptides, or single-chain fragments constructed in bacteriophage lambda or DNA or phagemids derived therefrom, or that would allow separate virus DNA arms to be generated in vivo following infection with a modified viral vector could greatly increase the quality and titer of the eukaryotic virus cDNA libraries or other libraries that are constructed using these methods.

Transfer of cDNA inserts from a Bacteriophage Lambda Library to Vaccinia Virus. Lambda phage vectors have several advantages over plasmid vectors for construction of cDNA libraries or other libraries, such as polynucleotides encoding intracellular immunoglobulin subunit polypeptides. Plasmid cDNA (or other DNA insert) libraries or linear DNA libraries are introduced into bacteria cells by chemical/heat shock transformation, or by electroporation. Bacteria cells are preferentially transformed by smaller plasmids, resulting in a potential loss of representation of longer cDNAs or other insert DNA, such as polynucleotides encoding intracellular immunoglobulin subunit polypeptides, in a library. In addition, transformation is a relatively inefficient process for introducing foreign DNA or other DNA into a cell requiring the use of expensive commercially prepared competent bacteria in order to construct a cDNA library or other library, such as polynucleotides encoding intracellular immunoglobulin subunit polypeptides or single-chain fragments. In contrast, lambda phage vectors can tolerate cDNA inserts of 12 kilobases or more without any size bias. Lambda vectors are packaged into virions in vitro using high efficiency commercially available packaging extracts so that the recombinant lambda genomes can be introduced into bacterial cells by infection. This results in primary libraries with higher titers and better representation of large cDNAs or other insert DNA, such as polynucleotides encoding intracellular immunoglobulin subunit polypeptides, than is commonly obtained in plasmid libraries.

To enable transfer of cDNA inserts or other insert DNA, such as polynucleotides encoding intracellular immunoglobulin subunit polypeptides or single-chain fragments, from a library constructed in a lambda vector to a eukaryotic virus vector such as vaccinia virus, the lambda vector must be modified to include vaccinia virus DNA sequences that allow for homologous recombination with the vaccinia virus DNA. The following example uses vaccinia virus homologous sequences, but other viruses may be similarly used. For example, the vaccinia virus HindIll J fragment (comprising the vaccinia tk gene) contained in plasmid p7.5/ATGO/tk (as described in Zauderer, WO 00/028016, published May 18, 2000, which is incorporated herein by reference in its entirety) can be excised using HindIII and SnaBI (3 kb of vaccinia DNA sequence), and subcloned into the HindIII/SnaBI sites of pT7Blue3 (Novagen cat no. 70025-3) creating pT7B3.Vtk. The vaccinia tk gene can be excised from this vector with SacI and SnaBI and inserted into the SacI/SmaI sites of Lambda Zap Express (Stratagene) to create lambda.Vtk. The lambda.Vtk vector will contain unique NotI, BamHI, SmaI, and SalI sites for insertion of cDNA downstream of the vaccinia 7.5k promoter. cDNA libraries can be constructed in lambda.Vtk employing methods that are well known in the art. DNA from a cDNA library or other library, such as polynucleotides encoding intracellular immunoglobulin subunit polypeptides, constructed in lambda.Vtk, or any similar bacteriophage that includes cDNA inserts or other insert DNA with flanking vaccinia DNA sequences to promote homologous recombination, can be employed to generate cDNA or other insert DNA recombinant vaccinia virus. Methods are well known in the art for excising a plasmid from the lambda genome by coinfection with a helper phage (ExAssist phage, Stratagene cat no. 211203). Mass excision from a lambda based library creates an equivalent cDNA library or other library in a plasmid vector. Plasmids excised from, for example, the lambda.Vtk cDNA library will contain the vaccinia tk sequences flanking the cDNA inserts or other insert DNAs, such as polynucleotides encoding intracellular immunoglobulin subunit polypeptides. This plasmid DNA can then be used to construct vaccinia recombinants by trimolecular recombination. Another embodiment of this method is to purify the lambda DNA directly from the initial lambda.Vtk library, and to transfect this recombinant viral (lambda) DNA or fragments thereof together with the two large vaccinia virus DNA fragments for trimolecular recombination.

Generation of vaccinia arms in vivo. Purification and transfection of vaccinia DNA or other virus DNA “arms” or fragments is a limiting factor in the construction of polynucleotide libraries by trimolecular recombination. Modifications to the method to allow for the requisite generation of virus arms, in particular vaccinia virus arms, in vivo would allow for more efficient construction of libraries in eukaryotic viruses.

Host cells can be modified to express a restriction endonuclease that recognizes a unique site introduced into a virus vector genome. For example, when a vaccinia virus infects these host cells, the restriction endonuclease will digest the vaccinia DNA, generating “arms” that can only be repaired, i.e., rejoined, by trimolecular recombination. Examples of restriction endonucleases include the bacterial enzymes NotI and ApaI, the Yeast endonuclease VDE (R. Hirata, Y. Ohsumi, A. Nakano, H. Kawasaki, K. Suzuki, Y. Anraku. 1990 J. Biological Chemistry 265: 6726-6733), the Chlamydomonas eugametos endonuclease I-CeuI and others well-known in the art. For example, a vaccinia strain containing unique NotI and ApaI sites in the tk gene has already been constructed, and a strain containing unique VDE and/or I-CeuI sites in the tk gene could be readily constructed by methods known in the art.

Constitutive expression of a restriction endonuclease would be lethal to a cell, due to the fragmentation of the chromosomal DNA by that enzyme. To avoid this complication, in one embodiment host cells are modified to express the gene(s) for the restriction endonuclease(s) under the control of an inducible promoter.

A preferred method for inducible expression utilizes the Tet-On Gene Expression System (Clontech). In this system expression of the gene encoding the endonuclease is silent in the absence of an inducer (tetracycline). This makes it possible to isolate a stably transfected cell line that can be induced to express a toxic gene, i.e., the endonuclease (Gossen, M. et al., Science 268: 1766-1769 (1995)). The addition of the tetracycline derivative doxycycline induces expression of the endonuclease. In a preferred embodiment, BSC1 host cells will be stably transfected with the Tet-On vector controlling expression of the NotI gene. Confluent monolayers of these cells will be induced with doxycycline and then infected with v7.5/tk (unique NotI site in tk gene), and transfected with cDNA or insert DNA recombinant transfer plasmids or transfer DNA or lambda phage or phagemid DNA. Digestion of exposed vaccinia DNA at the unique NotI site, for example, in the tk gene or other sequence by the NotI endonuclease encoded in the host cells produces two large vaccinia DNA fragments which can give rise to full-length viral DNA only by undergoing trimolecular recombination with the transfer plasmid or phage DNA. Digestion of host cell chromosomal DNA by NotI is not expected to prevent production of modified infectious viruses because the host cells are not required to proliferate during viral replication and virion assembly.

In another embodiment of this method to generate virus arms such as vaccinia arms in vivo, a modified vaccinia strain is constructed that contains a unique endonuclease site in the tk gene or other non-essential gene, and also contains a heterologous polynucleotide encoding the endonuclease under the control of the T7 bacteriophage promoter at another non-essential site in the vaccinia genome. Infection of cells that express the T7 RNA polymerase would result in expression of the endonuclease, and subsequent digestion of the vaccinia DNA by this enzyme. In a preferred embodiment, the v7.5/tk strain of vaccinia is modified by insertion of a cassette containing the cDNA encoding NotI with expression controlled by the T7 promoter into the HindIII C or F region (Coupar, E. H. B. et al., Gene 68: 1-10 (1988); Flexner, C. et al., Nature 330: 259-262 (1987)), generating v7.5/tk/T7NotI. A cell line is stably transfected with the cDNA encoding the T7 RNA polymerase under the control of a mammalian promoter as described (0. Elroy-Stein, B. Moss. 1990 Proc. Natl. Acad. Sci. USA 87: 6743-6747). Infection of this packaging cell line with v7.5/tk/T7NotI will result in T7 RNA polymerase dependent expression of NotI, and subsequent digestion of the vaccinia DNA into arms. Infectious full-length viral DNA can only be reconstituted and packaged from the digested vaccinia DNA arms following trimolecular recombination with a transfer plasmid or phage DNA. In yet another embodiment of this method, the T7 RNA polymerase can be provided by co-infection with a T7 RNA polymerase recombinant helper virus, such as fowlpox virus (P. Britton, P. Green, S. Kottier, K. L. Mawditt, Z. Penzes, D. Cavanagh, M. A. Skinner. 1996 J. General Virology 77: 963-967).

A unique feature of trimolecular recombination employing these various strategies for generation of large virus DNA fragments, preferably vaccinia DNA fragments in vivo is that digestion of the vaccinia DNA may, but does not need to precede recombination. It suffices that only recombinant virus escapes destruction by digestion. This contrasts with trimolecular recombination employing transfection of vaccinia DNA digested in vitro where, of necessity, vaccinia DNA fragments are created prior to recombination. It is possible that the opportunity for bimolecular recombination prior to digestion will yield a greater frequency of recombinants than can be obtained through trimolecular recombination following digestion.

Selection and Screening Strategies for Isolation of Recombinant Intracellular Immunoglobulin Molecules Using Virus Vectors, Especially Poxviruses. In certain embodiments of the present invention, the trimolecular recombination method is used in the production of libraries of polynucleotides expressing intracellular immunoglobulin subunit polypeptides or single-chain fragments. In this embodiment, libraries comprising full-length intracellular immunoglobulin subunit polypeptides, or preferably fragments thereof or single-chain fragments, are prepared by first inserting cassettes encoding immunoglobulin constant regions and signal peptides into a transfer plasmid which contains 5′ and 3′ regions homologous to vaccinia virus. Rearranged immunoglobulin variable regions are isolated by PCR from pre-B cells from unimunized animals of from B cells or plasma cells from immunized animals.

These PCR fragments may be cloned between, and in frame with the immunoglobulin signal peptide and constant region, to produce a coding region for an intracellular immunoglobulin subunit polypeptide. Preferrably, the PCR fragments are cloned into an immunoglobulin sequence which lack the signal sequence, and which may additionally lack a constant region. These transfer plasmids are introduced into host cells with poxvirus “arms,” and the tri-molecular recombination method is used to produce the libraries.

The present invention provides a variety of methods for identifying, i.e., selecting or screening for intracellular immunoglobulin molecules, or fragments thereof, can directly or indirectly induce a desired, predetermined modified phenotype in eukaryotic cells.

The selection and screening techniques of the present invention eliminate the bias imposed by selection of antibodies in rodents or the limitations of synthesis and assembly in bacteria.

Many of the identification methods described herein depend on expression of host cell genes or host cell transcriptional regulatory regions, which are directly or indirectly modified by intracellular immunoglobulin molecules, or fragments thereof. It is important to note that most preferred embodiments of the present invention require that host cells be infected with a eukaryotic virus vector, preferably a poxvirus vector, and even more preferably a vaccinia virus vector. It is well understood by those of ordinary skill in the art that some host cell protein synthesis is rapidly shut down upon poxvirus infection in some cell lines, even in the absence of viral gene expression. This problem is not intractable, however, because in certain cell lines, inhibition of host protein synthesis remains incomplete until after viral DNA replication. See Moss, B., “Poxviridae and their Replication” IN Virology, 2d Edition, B. N. Fields, D. M. Knipe et al., Eds., Raven Press, p. 2096 (1990). There is a need, however, to rapidly screen a variety of host cells for their ability to express gene products which are upregulated by an intracellular immunoglobulin molecule, or fragment thereof, upon infection by a eukaryotic virus vector, preferably a poxvirus vector, and even more preferably a vaccinia virus vector; and to screen desired host cells for differential expression of cellular genes upon virus infection with various mutant and attenuated viruses.

Accordingly, a method is provided for screening a variety of host cells for the expression of host cell genes and/or the operability of host cell transcriptional regulatory regions effecting a particular phenotype, upon infection by a virus vector, through expression profiling of particular host cells in microarrays of ordered cDNA libraries. Expression profiling in microarrays is described in Duggan, D. J., et al., Nature Genet. 21(1 Suppl):10-14 (1999), which is incorporated herein by reference in its entirety.

According to this method, expression profiling is used to compare host cell gene expression patterns in uninfected host cells and host cells infected with a eukaryotic virus expression vector, preferably a poxvirus vector, even more preferably a vaccinia virus vector, where the particular eukaryotic virus vector is the vector used to construct said first and said second libraries or said single-chain fragment library of polynucleotides of the present invention. In this way, suitable host cells which continue to undergo expression of the necessary inducible proteins upon infection with a given virus, can be identified.

Expression profiling is also used to compare host cell gene expression patterns in a given host cell, for example, comparing expression patterns when the host cell is infected with a fully infectious virus vector, and when the host cell is infected with a corresponding attenuated virus vector. Expression profiling in microarrays allows large-scale screening of host cells infected with a variety of attenuated viruses, where the attenuation is achieved in a variety of different ways known in the art or described herein.

Using this method, expression profiling in microarrays may be used to identify suitable host cells, suitable transcription regulatory regions, and/or suitable attenuated viruses in any of the selection/screening methods described herein.

Phenotypes. In certain embodiments, host cells expressing intracellular immunoglobulin molecules, or fragments thereof encoded by a library are identified by screening or selecting for a modified phenotype. Polynucleotides from those host cells exhibiting the modified phenotype are recovered. In certain embodiments, the intracellular immunoglobulin molecules, or fragments thereof, interfere with and/or bind an unknown antigen(s) (e.g., gene products) involved in producing a modified phenotype of interest and the intracellular immunoglobulin molecules, or fragments thereof, may be used to isolate and/or characterize the antigens, as described herein. Alternatively, intracellular immunoglobulin molecules, or fragments thereof, are identified which interfere with and/or bind a particular antigen of interest, using the two-hybrid system, as described herein.

By intracellular binding to target antigens it is possible to disrupt the normal functioning of antigens such as gene products (e.g., proteins, DNA, RNA) and therefore modify a phenotype. Examples of phenotypes that may be modified by intracellular immunoglobulin molecules, or fragments thereof, include the following.

For example, by binding to a protein that has to be further processed such as a receptor protein, a viral envelope protein, e.g. HIV gp160, can significantly reduce the cleavage of the protein into its active components. As another example, the capsid protein, e.g. the HIV capsid protein, is modified co-translationally by addition of the fatty acid, myristic acid. It appears that myristic acid is involved in the attachment of the capsid precursor protein to the inner surface of cells. In HIV proviruses which have been altered so that they are not capable of adding this myristic acid, the provirus is not infectious. Studies of the process of myristylation reveal a requirement for glycine at position two from the amino terminus and also at amino acid residues within six to ten amino acids from the site of myristylation. Thus, antibody binding to the protein at and near these sites can disrupt myristylation, and consequently modify a phenotype such as HIV infectivity.

Similarly, binding to a protein that has a significant external domain can hinder the effect of the protein.

In another embodiment, by binding to a dysfunctional receptor protein, one can block the undesired interactions that can result in cellular dysfunction such as malignant transformation.

For example, many proteins, such as surface receptors, transmembrane proteins, etc. are processed through the endoplasmic reticulum (sometimes referred to as ER-Golgi apparatus). Examples of such proteins include neu, envelope glycoproteins such as those of the primate lentiviruses, e.g., HIV or HIV-2. By using antibodies that can be delivered to such a region of the cell and be specific for a particular protein, one can disrupt the function of such protein without disrupting other cellular functions. For example, the PDGF-/2 and FGF-like factors produced by sis and int-2 pass through the ER. These factors are involved in many cancers. Thus, in addition to targeting the receptor, one can target the growth factors by using antibodies to them.

Growth factors are also expressed by many other malignant cells such as from carcinoid syndrome tumors and these would be another target.

One can also use this method to disrupt a function that is undesirable at a particular time. For example, the MHC class I and class II molecules are important in the immune system's recognition of antigens. [Teyton, L., et al., The New Biologist 4:441-447 (1992); Cox, J. H., et al., Science 247:715-718 (1990); Peters, P. J., et al., Nature 349:669-676 (1991); Hackett, Nature 349:655-656 (1991)]. However, such immune recognition, particularly from MHC class II molecules can cause problems such as in organ transplants. [Schreiner, G. F., et al., Science 240:10321033 (1988)]. Thus, by targeting class II molecules with organ transplants you can down regulate the host immune response. These molecules can preferably be targeted at different points in their processing pathway. Preferably, one would use an inducible promoter for the antibody gene.

Many variations of this method will be apparent to the skilled artisan.

For instance, the HIV-1 envelope gene directs the synthesis of a precursor polyglycoprotein termed gp160. This protein is modifiedby addition of multiple N-linked sugars as it enters the endoplasmic reticulum [Allan, J. S., et al., Science 228:1091-1094 (1985); Robey, W. G., Science 228:593-595 (1985); DiMarzo-Veronese, F., et al., Science 229:1402-1405 (1985); Willey, R. L., Cell Biol. 85:9580-9584 (1988)]. The glycosylated envelope protein precursor is then cleaved within the Golgi apparatus to yield a mature envelope protein comprised of an exterior glycoprotein, gp120, and a transmembrane protein, gp4l [Willey, Cell Biol, surpra; Stein, B. S., et al., J. Biol. Chem. 265:2640-2649 (1990); Earl, P. L., et al., J. Virol. 65:2047-2055 (1991)]. The envelope glycoprotein complex is anchored to the virion envelope and infects cell membranes by gp4l through non-covalent interactions [DiMarzo Veronese, Science, supra; Gelderblom, H. R., et al., Lancet ii:1016-1017 (1985)]. Following binding of the gp120 exterior glycoprotein to the CD4 receptor, the fusion of viral and host cell membranes allows virus entry [Stein, B. S., Cell 49:659-668 (1987)]. The fusogenic domain of the gp120/gp4l complex is thought to reside at the amino terminus of gp4l because this region exhibits sequence homology with a fusogenic domain of other viral proteins [Gallaher, W. R., Cell 50:327-328 (1987)]; Gonzalez-Scarano, F., AIDS Res. Hum. Retrovir. 3:245-252 (1987)) and because mutations in this region inactivate the virus and prevent viral fusion [Kowalski, M., et al., Science 237:1351-1355 (1987); Kowalski, M., et al., J. ViroL 65:281-291 (1991);

McCune, J. M., et al., Cell 53:55-67 (1988)).

While the processed gp120 and gp41 are transported to the cell surface and secreted as part of the virion, sometimes referred to as viral particles, the uncleaved gp160 is delivered to lysosomes for degradation. The cleavage process normally is relatively inefficient. Thus, the method of using intracellular antibodies to bind to the newly synthesized gp160 in the lumen of the endoplasmic reticulum and inhibit its transport to the Golgi apparatus greatly reduces the amount of protein available for cleavage to gp120 and gp41. Accordingly, the viral particles produced have greatly diminished amounts of gp120 and gp41 on their surface. Such particles are not considered as infectious.

This discussion of the HIV-1 gp160/120/41 proteins is exemplary of other envelope proteins and processed proteins. The same techniques used herein can be adapted by known techniques based upon the present disclosure.

Additionally, the envelope protein of the immunodeficiency viruses has been implicated in the other aspects of the disease such as membrane fusion, cell lysis, cytopathic effects, and syncytium formation [DeRossi, A., et al., Proc. Natl. Acad. Sci. 83:4297-4301 (1986)]. Intracellular expression of an antibody against HIV proteins such as the envelope protein reduced HIV infectivity, etc. (WO 94/02610).

Numerous regions of an antigen can be targeted by an intrabody, for example, targeting the cytoplasmic side of a membrane receptor. It is through the cytoplasmic tail that signal transduction occurs. See Luttrell, L. M. et al, Science 259:1453-1457 (1993); Epstein, R-J., et al., Proc. Natl. Acad. Sci USA 89:10435-10439 (1992). As an example, the neu/erbB-2 receptor or G protein receptor loop or cytoplasmic tail can be targeted, thereby preventing such signal transduction. Intracellular immunoglobulin molecules, or fragments thereof, may interfere with activated receptors such as phosphorylated amino acids on those activated receptors. Thus, the pool of target receptors can be reduced and a reduction in signal transduction is screened for or selected, by means that are well-known in the art.

Intracellular immunoglobulin molecules, or fragments thereof, may specifically bind to the antigen, e.g. a protein, and thus effectively compete with other molecules that would have normally formed complexes with the antigen.

The method is broadly applicable to a wide range of antigens including proteins, RNA, DNA, haptens, phospholipids, carbohydrates, etc. as will be discussed below.

Phenotypes which may be screened or selected for include adherence/nonadherence, growth suppression, growth stimulation, proliferation, apoptosis, cell lysis, cell integrity, cell viability, sensitization to an agent (e.g., small molecules, chemicals, biologicals, physical treatments, drugs, infective agent, a DNA-damaging agent, a therapeutic agent, etc.,) cytoskeletal function, ATP production, cell-disruption, expression of an antigen, cell differentiation, transformation., cell size, the expression of any number of moieties (including receptors, particularly cell surface receptors, adhesion molecules, antigens, e.g., cell-surface antigens, and cytokines), protein-protein interactions, transcriptional activation of particular promoters, etc. These and other cell phenotypes that may be screened for or selected are not mutually exclusive with one another and many may overlap. Thus, any modification of a phenotype such as enhancement or reduction in that phenotype compared to control cells, e.g., host cells, host cells containing the vector alone, and/or host cells containing the vector with an unrelated library as insert, are contemplated. Examples of screening and selection methods for these phenotypes are disclosed herein and may be found in U.S. application no. 60/203,343.

These modified phenotypes may be screened for or selected by many means. For example, enhanced expression of antigens may be screened for or selected by the following: antibody binding, and immune system-mediated disruption such as by CTLs, antibody-dependent cellular cytotoxicity (ADCC), and complement-dependent cytotoxicity (CDC). Reduced expression of antigens may be screened for or selected by a failure to bind antibody, failure to be disrupted by immune system-mediated mechanisms, etc.

Intrabodies may directly or indirectly induce a phenotypic modification by interfering with the gene products of intracellular infectious agents such as viruses. Thus, the intrabodies may lessen infectivity or lessen cytopathic or other effects of the infectious agent on cells and/or tissues. For example, intrabodies may interfere with viral gene products in HIV infected cells, such as the structural proteins envelope glycoprotein and gag protein, and/or tat, rev, nef, vpu and/or vpx regulatory proteins, and/or the nucleic acid binding site TAR. Intrabodies that recognize particular HIV proteins have been shown to interfere with HIV infectivity and effects of HIV infection (WO 94/02610).

Intrabodies may also directly or indirectly induce a phenotypic modification by interfering with a cell receptor for an infectious agent. Thus, the intrabodies may down-regulate cell surface expression of a receptor or may interfere with entry of the infectious agent via the receptor. For example, it has been shown that an intrabody specific for the HIV co-receptor CCR5 reduces HIV infectivity and effects of HIV infection. (Steinberger, et al. PNAS USA 97:805-810 (2000).

The method can be used to select intracellular immunoglobulin molecules, or fragments thereof, which sensitize host cells to killing by an agent. In this embodiment, the host cells are exposed to a compound which induces death in a cell expressing an intracellular immunoglobulin molecule, or fragment thereof. Following cell death, intracellular debris and nonviable cells containing the library polynucleotide may be removed from the cell culture, thereby recovering the polynucleotide.

Additionally, cells may be screened or selected, for example, by expression of a reporter gene. The reporter gene may be under the control of a non-constitutive promoter, and preferably is under the control of an inducible promoter. Examples of non-constitutive or inducible promoters include a differentiation-induced promoter, a cell type-restricted promoter, a tissue-restricted promoter, a temporally-regulated promoter, a spatially-regulated promoter, a proliferation-induced promoter, a cell-cycle specific promoter. For example, the promoter may be a promoter induced during differentiation of musculoskeletal cells, as described in the Examples. Reporter genes and suicide genes are disclosed herein and disclosed in U.S. application no. 60/203,343.

Additionally, cells may be screened or selected, for example, by fluorescence-activated cell sorting (FACS). Fluorescence activated cell sorting (FACS), also called flow cytometry, is used to sort individual cells on the basis of optical properties, including fluorescence.

Intracellular immunoglobulin molecules, or fragments thereof may interfere with cell proliferation regulators which, when aberrantly expressed or regulated, may induce or otherwise be involved in the development of cell proliferative disorders. Such cell proliferative disorders include, but are not limited to cancers, arteriosclerosis, psoriasis, viral disease, as well as inflammatory conditions such as arthritis or sepsis. Cell proliferation genes include dominant transforming genes, such as oncogenes and other genes encoding products involved in the induction of cell growth and recessive cell proliferation genes, such as genes encoding tumor suppressors, genes involved in the induction of apoptosis or genes involved in viral growth.

Intracellular immunoglobulin molecules, or fragments thereof may modulate cell cycle regulation, by, for example, suppressing or activating a cell cycle checkpoint pathway, or ameliorating or inducing checkpoint defects. Thus, in a preferred embodiment, host cells are sorted in a FACS machine by assaying cell parameters, including, but not limited to, cell viability, cell proliferation, and cell phase. In this embodiment, preferred cellular parameters or assays are cell viability assays, assays to determine whether cells are arrested at a particular cell cycle stage (“cell proliferation assays”), and assays to determine at which cell stage the cells have arrested (“cell phase assays”). By assaying or measuring one or more of these parameters, it is possible to detect not only alterations in cell cycle regulation, but alterations of different steps of the cell cycle regulation pathway. In this manner, rapid, accurate screening of intracellular immunoglobulin molecules, or fragments thereof, may be performed to identify those that modulate cell cycle regulation, viability, growth, proliferation, etc. It may be possible to alter the activities of certain enzymes, for example kinases, phosphatases, proteases or ubiquitination enzymes, that contribute to initiating cell phase and/or other changes.

In certain embodiments, the methods are used to evaluate cell cycle regulation. Cells cycle through various stages of growth, starting with the M phase, where mitosis and cytoplasmic division (cytokinesis) occurs. The M phase is followed by the G1 phase, in which the cells resume a high rate of biosynthesis and growth. The S phase begins with DNA synthesis, and ends when the DNA content of the nucleus has doubled. The cell then enters G2 phase, which ends when mitosis starts, signaled by the appearance of condensed chromosomes. Terminally differentiated cells are arrested in the G1 phase, and no longer undergo cell division. In this embodiment, preferredcellularparameters or assays are cell viability assays, assays to determine whether cells are arrested at a particular cell cycle stage (“cell proliferation assays”), and assays to determine at which cell stage the cells have arrested (“cell phase assays”). By separating or screening cells based on one or more of these parameters, it is possible to detect not only alterations in cell cycle regulation, but alterations of different steps of the cell cycle regulation pathway, and to isolate polynucleotides encoding intracellular immunoglobulin molecules, or fragments thereof, which confer such alteration.

In a preferred embodiment, the methods outlined herein are performed on cells that are not arrested in the G1 phase; that is, they are rapidly or uncontrollably growing and replicating, such as tumor cells. In this manner, intracellular immunoglobulin molecules, or fragments thereof are evaluated to target polynucleotides that alter cell cycle regulation, i.e. cause cells to arrest at cell cycle checkpoints, such as G1, although arresting in other phases such as S, G2 or M are also desirable. Alternatively, intracellular immunoglobulin molecules, or fragments thereof are evaluated to find those that cause proliferation of a population of cells, i.e. that allow cells that are generally arrested in G1 to start proliferating again; for example, peripheral blood cells, terminally differentiated cells, stem cells in culture, etc.

A host cell containing a polynucleotide encoding an intracellular immunoglobulin subunit polypeptide may become “nonadherent” or “nonviable” by any mechanism, which may include lysis, inability to adhere, loss of viability, loss of membrane integrity, loss of structural stability, disruption of cytoskeletal elements, inability to maintain membrane potential, arrest of cell cycle, inability to generate energy, etc. Thus, host cells containing target polynucleotides may be recovered, i.e., separated from remaining cells, by any physical means such as aspiration, washing, filtration, centrifugation, cell sorting, fluorescence activated cell sorting (FACS), etc.

For example, host cells containing polynucleotides encoding intracellular immunoglobulin subunit polypeptides may lyse and thereby release recombinant virus particles, preferably poxvirus particles even more preferably vaccinia virus particles into the culture media or may become nonadherent and therefore lift away from the solid support. Thus, in a preferred embodiment, released recombinant viruses and/or nonadherent cells are separated from adherent cells by aspiration or washing.

In certain embodiments, intracellular immunoglobulin molecules, or fragments thereof which bind a particular antigen are screened for or selected for. Particularly preferred are the methods described in the Examples and elsewhere herein, comprising two-hybrid systems. In such embodiments, intrabody-antigen-induced cell death is effected directly or indirectly by employing a host cell transfected with a construct in which a foreign polynucleotide, the expression of which indirectly results in cell death, is operably associated with a transcriptional regulatory region which is induced upon activation of two-hybrid transcriptional regulator.

By a “transcriptional regulatory region induced upon activation of two-hybrid transcriptional regulator” is meant a region, for example, a host cell or other promoter, which is activated by a transcriptional activator domain which is part of a two-hybrid system, such as those as described in the Examples and known in the art (Portner-Taliana, et al., J. Immun. Meth. 238:161-171 (2000); Visintin, et al., PNAS USA 96:11723 (1999); Clontech Matchmaker™ System, Palo Alto, Calif.; Invitrogen, Carlsbad, Calif.).

Expression of a transcriptional regulatory region may be screened or selected in any appropriate means described herein or otherwise known in the art. For example, a reporter gene (e.g., CAT, luciferase, etc.), suicide gene, polynucleotide encoding an endogenous antigen (e.g., cell surface antigen), or polynucleotide encoding an endogenous CTL antigen, may be operably associated with (e.g. under the control of) the transcriptional regulatory region. Thus, activation of the transcriptional regulatory region which is part of a two-hybrid system induces expression of a reporter gene, suicide gene, etc, which expression may be screened for or selected.

The two hybrid system is based on the fact that many eukaryotic transcriptional activators are comprised of two physically and functionally separable domains, a DNA-binding domain (DNA-BP) and an activation domain (AD). The two domains are normally part of the same protein. However, the two domains can be separated and expressed as distinct proteins. Two additional proteins (X and Y) are expressed as fusions to the DNA-BP and AD peptides. If X and Y interact, the AD is co-localized to the DNA-BP bound to the transcriptional regulatory region, resulting in transcription from that region. If X is an intrabody specific for an antigen, for example, a member of an intrabody library, and Y is that antigen, then transcription will occur. If the transcriptional regulatory region is in operably association with, for example, a reporter gene, suicide gene, or polynucleotide encoding an antigen, etc., then cells expressing intrabodies which recognize Y can be screened for or selected.

In the present invention, intrabodies which recognize (e.g., interfere with or bind with) an antigen of choice are selected or screened for using a two-hybrid system. These embodiments involve three types of constructs. (A) The first or second library, or the single-chain fragment library, comprises a polynucleotide encoding an AD, in-frame with the reading frame of the intracellular immunoglobulin molecule, or fragment thereof (known in the art as the “prey” construct). (B) A polynucleotide encoding the antigen of choice is fused in-frame with a polynucleotide encoding a DNA-BP (known in the art as the “bait” construct). (C) A reporter gene (or suicide gene, in the present invention) in operable association with a transcriptional regulatory region (e.g, a DNA binding domain recognized by the DNA-BP and a minimal promoter) (known in the art as the “reporter” construct).

The AD may be from, for example, VP16 or LexA. The DNA-BP may be from, for example, LexA or GAL4. The transcriptional regulatory region corresponds to the DNA-BP. Construct (A) or (B) may include one or more nuclear localization sequences, and preferably construct (A) (i.e., the intrabody library-AD construct) comprises one or more nuclear localization sequences. Any of the constructs, particularly (A) and/or (B) may also contain a linker joining the immunoglobulin and other polypeptides/domains which are fused, or joining the localization sequences which are fused to the immunoglobulin and/or other polypeptide/domains.

The (A), (B) and (C) constructs may be made in any appropriate vectors and introduced into host cells by any appropriate method. In preferred embodiments, contruct (A) is in a poxvirus vector, preferably vaccinia virus, and constructs (B) and (C) are in plasmid vectors. In other preferred embodiments, (A), (B), and (C) are made in poxvirus vectors, preferably vaccinia virus.

In one embodiment, a method is provided to induce cell death upon expression of a foreign polynucleotide encoding a cytotoxic T cell (CTL) epitope. The foreign polynucleotide encoding the CTL epitope is placed in operable association with a transcriptional regulatory region which is induced upon expression of an intracellular immunoglobulin molecule or fragment thereof. The polynucleotide encoding the CTL epitope may be under the control of a cell- or tissue- or other non-constitutive endogenous promoter, or may be under the control of a transcription regulatory sequence as part of a two-hybrid system, as described in the Examples. Upon expression of a desired intracellular immunoglobulin molecule or fragment thereof, the CTL epitope is expressed on the surface of the host cell in the context of a defined MHC molecule which is also expressed on the surface of the host cell. The cells are contacted with epitope-specific CTLs which recognize the CTL epitope in the context of the defined MHC molecule, and the cells expressing the CTL epitope rapidly undergo a lytic event. Methods of selecting and recovering host cells expressing specific CTL epitopes are further disclosed in Zauderer, PCT Publication No. WO 00/028016.

Selection of the host cells is accomplished through recovering those cells, or the contents thereof, which have succumbed to cell death and/or have undergone a lytic event. For example, if host cells are chosen which grow attached to a solid support, those host cells which succumb to cell death and/or undergo a lytic event will be released from the support and can be recovered in the cell supernatant. Alternatively virus particles released from host cells which have succumbed to cell death and/or undergone a lytic event may be recovered from the cell supernatant.

According to this embodiment, the MHC molecule expressed on the surface of the host cells may be either a class I MHC molecule or a class II MHC molecule. In a particularly preferred embodiment, the MHC molecule expressed on the host cells is an H-2K^{d} molecule, and the CTL epitope which is expressed is the peptide GYKAGMIHI, designated herein as SEQ ID NO:77.

In another preferred embodiment, a method is provided wherein cell death is induced indirectly by employing a host cell transfected with a construct in which the a heterologous polynucleotide comprising a “suicide” gene is operably associated with a transcriptional regulatory region which is directly or indirectly induced upon expression of an intracellular immunoglobulin molecule, or fragment thereof. The suicide gene may be under the control of a cell- or tissue- or other non-constitutive endogenous promoter, or may be under the control of a transcription regulatory sequence as part of a two-hybrid system, as described in the Examples. By “suicide gene” is meant a nucleic acid molecule which causes cell death when expressed. Polynucleotides useful as suicide genes include many cell death-inducing sequences which are known in the art. Preferred suicide genes are those which encode toxins such as Pseudomonas exotoxin A chain, diphtheria A chain, ricin A chain, abrin A chain, modeccin A chain, and alpha-sarcin. A preferred suicide gene encodes the diphtheria A toxin subunit. Upon expression of an intracellular immunoglobulin molecule, or fragment thereof the promoter of the suicide gene is induced, thereby allowing expression of the suicide gene, and thereby promoting cell death.

In another embodiment, a screening method is provided to recover polynucleotides encoding intracellular imrnmunoglobulin molecules, or fragments thereof based on expression of a reporter gene. The reporter gene may be under the control of a cell- or tissue- or other non-constitutive endogenous promoter, or may be under the control of a transcription regulatory sequence as part of a two-hybrid system. According to this method, host cells are transfected with an easily detected reporter construct, for example luciferase, operably associated with a promoter transcriptional regulatory region which is directly or indirectly upregulated or downregulated as a result of expression of an intracellular immunoglobulin molecule, or fragment thereof. Pools of host cells expressing intracellular immunoglobulin molecules, or fragments thereof, are screened or selected for the signal or lack thereof detected in that pool.

Any suitable reporter molecule may be used in these methods, the choice depending upon the host cells used, the detection instruments available, and the ease of detection desired. Suitable reporter molecules include, but are not limited to luciferase, green fluorescent protein, and beta-galactosidase.

Similar to the cell death methods described above, kinetic considerations dictate that expression of the reporter construct take place prior to the induction of CPE. Nonetheless, it is preferred that expression of a detectable reporter molecule occurs within a period between about 1 hour to about 4 days after, for example, introduction of the library, or contacting host cells with immune system-mediated effector or an agent, so as to precede induction of CPE. More preferably, reporter molecule expression occurs within about 1 hour about 2 hours, about 3 hours about 4 hours, about 5 hours, about 6 hours, about 7 hours, about 8 hours, about 9 hours, about 10 hours, about 11 hours, about 12 hours, about 14 hours, about 16 hours, about 18 hours, about 20 hours, about 22 hours, about 24 hours, about 28 hours, about 32 hours, about 36 hours, about 40 hours, about 44 hours, or about 48 hours after contacting the host cells with antigen. Even more preferably reporter molecule expression occurs within about 12 hours of, for example, introducing the library, etc.

As used herein, a “solid support” or a “solid substrate” is any support capable of binding a cell or antigen, which may be in any of various forms, as is known in the art. Well-known supports include tissue culture plastic, glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration as long as the coupled molecule is capable of binding to a cell. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. The support configuration may include a tube, bead, microbead, well, plate, tissue culture plate, petri plate, microplate, microtiter plate, flask, stick, strip, vial, paddle, etc., etc. A solid support may be magnetic or non-magnetic. Those skilled in the art will know many other suitable carriers for binding cells or antigens, or will be able to readily ascertain the same.

In a preferred embodiment, the present methods are useful in cardiovascular applications. In a preferred embodiment, cardiomyocytes may be screened for the prevention of cell damage or death in the presence of normally injurious conditions, including, but not limited to, the presence of toxic drugs (particularly chemotherapeutic drugs), for example, to prevent heart failure following treatment with adriamycin; anoxia, for example in the setting of coronary artery occlusion; and autoimmune cellular damage by attack from activated lymphoid cells (for example as seen in post viral myocarditis and lupus). Libraries of polynucleotides encoding intracellular immunoglobulin molecules, or fragments thereof, are introduced into cardiomyocytes, the cells are subjected to the insult, and intracellular immunoglobulin molecules, or fragments thereof are selected that prevent any or all of: apoptosis; membrane depolarization (e.g. decrease arrythmogenic potential of insult); swelling; or leakage of specific intracellular ions, second messengers and activating molecules (for example, arachidonic acid and/or lysophosphatidic acid).

In a preferred embodiment, the present methods are used to screen for diminished arrhythmia potential in cardimyocytes. The screens comprise the introduction of libraries of the present invention, followed by the application of arrythmogenic insults, with screening for intracellular immunoglobulin molecules, or fragments thereof that block specific depolarization of cell membrane. This may be detected using patch clamps, or via fluorescence techniques). Similarly channel activity (for example, potassium and chloride channels) in cardiomyocytes could be regulated using the present methods in order to enhance contractility and prevent or diminish arrhythmia.

In a preferred embodiment, the present methods are used to screen for enhanced contractile properties of cardiomyocytes and diminish heart failure potential. The introduction of the libraries of the invention followed by measuring the rate of change of myosin polymerization/depolymerization using fluorescent techniques can be done. Intracellular immunoglobulin molecules, or fragments thereof which increase the rate of change of this phenomenon can result in a greater contractile response of the entire myocardium, similar to the effect seen with digitalis.

In a preferred embodiment, the present methods are useful to identify intracellular immunoglobulin molecules, or fragments thereof that will regulate the intracellular and sarcolemmal calcium cycling in cardiomyocytes in order to prevent arrhythmias. Intracellular immunoglobulin molecules, or fragments thereof are selected that regulate sodium-calcium exchange, sodium proton pump function, and regulation of calcium-ATPase activity.

In a preferred embodiment, the present methods are useful to identify intracellular immunoglobulin molecules, or fragments thereof that diminish embolic phenomena in arteries and arterioles leading to strokes (and other occlusive events leading to kidney failure and limb ischemia) and angina precipitating a myocardial infarct are selected. For example, intracellular immunoglobulin molecules, or fragments thereof which will diminish the adhesion of platelets and leukocytes, and thus diminish the occlusion events. Adhesion in this setting can be inhibited by the libraries of the invention being introduced into endothelial cells (quiescent cells, or activated by cytokines, e.g. IL-1, and growth factors, e.g. PDGF/EGF) and then screening for intracellular immunoglobulin molecules, or fragments thereof that 1) down regulate adhesion molecule expression on the surface of the endothelial cells (binding assay); 2) block adhesion molecule activation on the surface of these cells (signaling assay); or 3) release in an autocrine manner peptides that block receptor binding to the cognate receptor on the adhering cell.

Embolic phenomena can also be addressed by activating proteolytic enzymes on the cell surfaces of endothelial cells, and thus releasing active enzyme which can digest blood clots. Thus, delivery of the libraries of the invention to endothelial cells is done, followed by standard fluorogenic assays, which will allow monitoring of proteolytic activity on the cell surface towards a known substrate. Intracellular immunoglobulin molecules, or fragments thereof can then be selected which activate specific enzymes towards specific substrates.

In a preferred embodiment, arterial inflammation in the setting of vasculitis and post-infarction can be regulated by decreasing the chemotactic responses of leukocytes and mononuclear leukocytes. This can be accomplished by blocking chemotactic receptors and their responding pathways on these cells. Libraries can be introduced into these cells, and the chemotactic response to diverse chemkines (for example, to the IL-8 family of chemokines, RANTES) is inhibited in cell migration assays.

In a preferred embodiment, arterial restenosis following coronary angioplasty can be controlled by regulating the proliferation of vascular intimal cells and capillary and/or arterial endothelial cells. Candidate intracellular immunoglobulin libraries can be introduced into these cell types and their proliferation in response to specific stimuli monitored. One application may be intracellular peptides which block the expression or function of c-myc and other oncogenes in smooth muscle cells to stop their proliferation. A second application may involve the expression of libraries in vascular smooth muscle cells to selectively induce their apoptosis. Application of therapeutics derived from these intracellular immunoglobulin molecules, or fragments thereof may require targeted drug delivery; this is available with stents, hydrogel coatings, and infusion-based catheter systems. Intracellular immunoglobulin molecules, or fragments thereof which down regulate endothelin-1A receptors or which block the release of the potent vasoconstrictor and vascular smooth muscle cell mitogen endothelin-1 may also be candidates for therapeutics. Intracellular immunoglobulin molecules, or fragments thereof can be isolated from these libraries which inhibit growth of these cells, or which prevent the adhesion of other cells in the circulation known to release autocrine growth factors, such as platelets (PDGF) and mononuclear leukocytes.

The control of capillary and blood vessel growth is an important goal in order to promote increased blood flow to ischemic areas (growth), or to cut-off the blood supply (angiogenesis inhibition) of tumors. Candidate intracellular immunoglobulin libraries can be introduced into capillary endothelial cells and cell growth monitored. Stimuli such as low oxygen tension and varying degrees of angiogenic factors can regulate the responses, and intracellular immunoglobulin molecules, or fragments thereof isolated that produce the appropriate phenotype. Screening for antagonism of vascular endothelial cell growth factor, important in angiogenesis, would also be useful.

In a preferred embodiment, the present methods are useful in screening for decreases in atherosclerosis-producing mechanisms to find intracellular immunoglobulin molecules, or fragments thereof that regulate LDL and HDL metabolism. Libraries can be introduced into the appropriate cells (including hepatocytes, mononuclear leukocytes, endothelial cells) and peptides selected which lead to a decreased release of IDL or diminished synthesis of LDL, or conversely to an increased release of HDL or enhanced synthesis of HDL. Intracellular immunoglobulin molecules, or fragments thereof can also be isolated from libraries which decrease the production of oxidized LDL, which has been implicated in atherosclerosis and isolated from atherosclerotic lesions. This could occur by decreasing its expression, activating reducing systems or enzymes, or blocking the activity or production of enzymes implicated in production of oxidized LDL, such as 15-lipoxygenase in macrophages.

In a preferred embodiment, the present methods are used in screens to regulate obesity via the control of food intake mechanisms or diminishing the responses of receptor signaling pathways that regulate metabolism. Intracellular immunoglobulin molecules, or fragments thereof that regulate or inhibit the responses of neuropeptide Y (NPY), cholecystokinin and galanin receptors, are particularly desirable. Libraries can be introduced into cells that have these receptors cloned into them, and intracellular immunoglobulin molecules, or fragments thereof, selected that block the signaling responses to galanin and NPY. In a similar manner, intracellular immunoglobulin molecules, or fragments thereof, can be found that regulate the leptin receptor.

In a preferred embodiment, the present methods are useful in neurobiology applications. Libraries may be used for screening for anti-apoptotics for preservation of neuronal function and prevention of neuronal death. Initial screens would be done in cell culture. One application would include prevention of neuronal death, by apoptosis, in cerebral ischemia resulting from stroke. Apoptosis is known to be blocked by neuronal apoptosis inhibitory protein (NAIP); screens for its upregulation, or effecting any coupled step could yield intracellular immunoglobulin molecules, or fragments thereof which selectively block neuronal apoptosis. Other applications include neurodegenerative diseases such as Alzheimer's disease and Huntington's disease.

In a preferred embodiment, the present methods are useful in bone biology applications. Osteoclasts are known to play a key role in bone remodeling by breaking clown “old” bone so that osteoblasts can lay down “new” bone. In osteoporosis one has an imbalance of this process. Osteoclast overactivity can be regulated by inserting libraries into these cells, and then looking for intracellular immunoglobulin molecules, or fragments thereof that produce: 1) a diminished processing of collagen by these cells; 2) decreased pit formation on bone chips; and 3) decreased release of calcium from bone fragments.

The present methods may also be used to screen for agonists of bone morphogenic proteins, hormone mimetics to stimulate, regulate, or enhance new bone formation (in a manner similar to parathyroid hormone and calcitonin, for example). These have use in osteoporosis, for poorly healing fractures, and to accelerate the rate of healing of new fractures. Furthermore, cell lines of connective tissue origin can be treated with libraries and screened for their growth, proliferation, collagen stimulating activity, and/or proline incorporating ability or change in production of collagen or bone.

In a preferred embodiment, the present methods are useful in skin biology applications. Keratinocyte responses to a variety of stimuli may result in psoriasis, a proliferative change in these cells. Libraries can be introduced into cells removed from active psoriatic plaques, and intracellular immunoglobulin molecules, or fragments thereof isolated which decrease the rate of growth of these cells.

In a preferred embodiment, the present methods are useful in the regulation or inhibition of keloid formation (e.g. excessive scarring). Libraries introduced into skin connective tissue cells isolated front individuals with this condition, and intracellular immunoglobulin molecules, or fragments thereof isolated that decrease proliferation, collagen formation, or proline incorporation.

Results from this work can be extended to treat the excessive scarring that also occurs in burn patients. Intracellular immunoglobulin molecules, or fragments thereof that inhibit one or more of these activities can be used widely in a topical manner to diminish scarring post burn.

Similarly, wound healing for diabetic ulcers and other chronic “failure to heal” conditions in the skin and extremities can be regulated by providing additional growth signals to cells which populate the skin and dermal layers. Growth factor mimetic may in fact be very useful for this condition. Libraries can be introduced into skin connective tissue cells, and intracellular immunoglobulin molecules, or fragments thereof isolated which promote the growth of these cells under “harsh” conditions, such as low oxygen tension, low pH, and the presence of inflammatory mediators.

Cosmeceutical applications of the present invention include the control of melanin production in skin melanocytes. A naturally occurring peptide, arbutin, is a tyrosine hydroxylase inhibitor, a key enzyme in the synthesis of melanin. Libraries can be introduced into melanocytes and known stimuli that increase the synthesis of melanin applied to the cells. Intracellular immunoglobulin molecules, or fragments thereof can be isolated that inhibit the synthesis of melanin under these conditions.

In a preferred embodiment the present methods are useful in endocrinology applications. The intracellular immunoglobulin library technology can be applied broadly to any endocrine, growth factor, cytokine or chemokine network which involves a signaling peptide or protein that acts in either an endocrine paracrine or autocrine manner that binds or dimerizes a receptor and activates a signaling cascade that results in a known phenotypic or functional outcome. The methods are applied so as to isolate a peptide which inhibits the hormone (e.g., insulin, leptin, calcitonin, PDGF, EGF, EPO, GMCSF, IL1-17, mimetics) by either blocking the release of the hormone, blocking its specific receptor or carrier protein (for example, CRF binding protein), or inhibiting the intracellular responses of the specific target cells to that hormone. This could have broad applications to conditions of hormonal deficiency.

In a preferred embodiment, the present methods are useful in infectious disease applications. Viral latency (herpes viruses such as CMV, EBV, HBV, and other viruses such as HIV) and their reactivation are a significant problem, particularly in immunosuppressed patients (e.g., patients with AIDS and transplant patients). The ability to block the reactivation and spread of these viruses is an important goal. Cell lines known to harbor or be susceptible to latent viral infection can be infected with the specific virus, and then stimuli applied to these cells which have been shown to lead to reactivation and viral replication. This can be followed by measuring viral liters in the medium and scoring cells for phenotypic changes. Libraries can then be introduced into these cells under the above conditions, and intracellular immunoglobulin molecules, or fragments thereof isolated which block or diminish the growth and/or release of the virus. As with chemotherapeutics, these experiments can also be done with drugs which are only partially effective towards this outcome, and intracellular immunoglobulin molecules, or fragments thereof isolated which enhance the virucidal effect of these drugs.

One example of many is the ability to block HIV-1 infection. HIV-1 requires CD4 and a co-receptor which can be one of several seven transmembrane G-protein coupled receptors. In the case of the infection of macrophages, CCR-5 is the required co-receptor, and a block on CCR-5 will result in resistance to HIV-1 infection. One introduces a cell line that expresses CCR-5 with a library of the invention. Using an antibody to CCR-5 one can use FACS to sort desired cells based on the binding of this antibody to the receptor. All cells which do not bind the antibody will be assumed contain inhibitors of this antibody binding site. These inhibitors, in the library can be further assayed for their ability to inhibit HIV-1 entry.

Viruses are known to enter cells using specific receptors to bind to cells (for example, HIV uses CD4, coronavirus uses CD 13, murine leukemia virus uses transport protein, and measles virus uses CD44) and to fuse with cells (HIV uses chemokine receptor). Libraries can be introduced into target cells known to be permissive to these viruses, and intracellular immunoglobulin molecules, or fragments thereof isolated which block the ability of these viruses to bind and fuse with specific target cells.

In a preferred embodiment, the present invention finds use with infectious organisms. Intracellular organisms such as mycobacteria, listeria, salmonella, pneumocystis, yersinia, leishmania, and T. cruzi, can persist and replicate within cells and become active in immunosuppressed patients. There are currently drugs on the market and in development which are either only partially effective or ineffective against these organisms. Libraries can be introduced into specific cells infected with these organisms (pre- or post-infection), and immunoglobulin molecules, or fragments thereof selected which promote the intracellular destruction of these organisms in a manner analogous to intracellular “antibiotic peptides” similar to magainins. In addition, intracellular immunoglobulin molecules, or fragments thereof can be selected which enhance the cidal properties of drugs already under investigation which have insufficient potency by themselves, but when combined with a specific intracellular immunoglobulin molecule or fragment thereof from a candidate library, are dramatically more potent through a synergistic mechanism. Finally, intracellular immunoglobulin molecules, or fragments thereof can be isolated which alter the metabolism of these intracellular organisms, in such a way as to terminate their intracellular life cycle by inhibiting a key organismal event.

Antibiotic drugs that are widely used have certain dose dependent, tissue specific toxicities. For example renal toxicity is seen with the use of gentamicin, tobramycin, and amphotericin; hepatotoxicity is seen with the use of INH and rifampin; bone marrow toxicity is seen with chloramphenicol; and platelet toxicity is seen with ticarcillin, etc. These toxicities limit their use. Libraries can be introduced into the specific cell types where specific changes leading to cellular damage or apoptosis by the antibiotics are produced, and intracellular immunoglobulin molecules, or fragments thereof can be isolated that confer protection when these cells are treated with these specific antibiotics.

Furthermore, the present invention finds use in screening for intracellular immunoglobulin molecules, or fragments thereof that block antibiotic transport mechanisms. The rapid secretion from the blood stream of certain antibiotics limits their usefulness. For example penicillins are rapidly secreted by certain transport mechanisms in the kidney and choroid plexus in the brain. Probenecid is known to block this transport and increase serum and tissue levels. Candidate intracellular immunoglobulin molecules, or fragments thereof can be introduced into specific cells derived from kidney cells and cells of the choroid plexus known to have active transport mechanisms for antibiotics. intracellular immunoglobulin molecules, or fragments thereof can then be isolated which block the active transport of specific antibiotics and thus extend the serum halflife of these drugs.

In a preferred embodiment, the present methods are useful in drug toxicities and drug resistance applications. Drug toxicity is a significant clinical problem. This may manifest itself as specific tissue or cell damage with the result that the drug's effectiveness is limited. Examples include myeloablation in cancer chemotherapy, damage to epithelial cells lining the airway and gut, and hair loss. Specific examples include adriamycin induced cardiomyocyte death, cisplatinin-induced kidney toxicity, vincristine-induced gut motility disorders, and cyclosporin induced kidney damage. Libraries can be introduced into specific cell types with characteristic drug-induced phenotypic or functional responses, in the presence of the drugs, and intracellular immunoglobulin molecules, or fragments thereof isolated which reverse or protect the specific cell type against the toxic changes when exposed to the drug. These effects may manifest as blocking the drug induced apoptosis of the cell of interest, thus initial screens will be for survival of the cells in the presence of high levels of drugs or combinations of drugs used in combination chemotherapy.

Drug toxicity may be due to a specific metabolite produced in the liver or kidney which is toxic to specific cells, or due to drug interactions in the liver which block or enhance the metabolism of an administered drug. Libraries can be introduced into liver or kidney cells following the exposure of these cells to the drug known to produce the toxic metabolite. The active intracellular immunoglobulin molecules, or fragments thereof can be isolated which alter how the liver or kidney cells metabolize the drug, and specific intracellular immunoglobulin molecules, or fragments thereof identified which prevent the generation of a specific toxic metabolite. The generation of the metabolite can be followed by mass spectrometry and phenotypic changes can be assessed by microscopy. Such a screen can also be done in cultured hepatocytes, cocultured with readout cells which are specifically sensitive to the toxic metabolite. Applications include reversible (to limit toxicity) inhibtors of enzymes involved in drug metabolism.

Multiple drug resistance, and hence tumor cell selection, outgrowth, and relapse, leads to morbidity and mortality in cancer patients. Libraries can be introduced into tumor cell lines (primary and cultured) that have demonstrated specific or multiple drug resistance. Intracellular immunoglobulin molecules, or fragments thereof can then be identified which confer drug sensitivity when the cells are exposed to the drug of interest, or to drugs used in combination chemotherapy. The readout can be the onset of apoptosis in these cells, membrane permeability changes, the release of intracellular ions and fluorescent markers. The cells in which multidrug resistance involves membrane transporters can be preloaded with fluorescent transporter substrates, and selection carried out for peptides which block the normal efflux of fluorescent drug from these cells. Libraries are particularly suited to screening for intracellular immunoglobulin molecule or fragment thereof which reverse poorly characterized or recently discovered intracellular mechanisms of resistance or mechanisms for which few or no chemosensitizers currently exist, such as mechanisms involving LRP (lung resistance protein). This protein has been implicated in multidrug resistance in ovarian carcinoma, metastatic malignant melanoma, and acute myeloid leukemia. Particularly interesting examples include screening for intracellular immunoglobulin molecules, or fragments thereof which reverse more than one important resistance mechanism in a single cell, which occurs in a subset of the most drug resistant cells, which are also important targets. Applications would include screening for peptide inhibitors of both MRP (multidrug resistance related protein) and LRP for treatment of resistant cells in metastatic melanoma, for inhibitors of both p-glycoproicin and LRP in acute myeloid leukemia, and for inhibition (by any mechanism) of all three proteins for treating pan-resistant cells.

In a preferred embodiment, the present methods are useful in improving the performance of existing or developmental drugs. First pass metabolism of orally administered drugs limits their oral bioavailability, and can result in diminished efficacy as well as the need to administer more drug for a desired effect. Reversible inhibitors of enzymes involved in first pass metabolism may thus be a useful adjunct enhancing the efficacy of these drugs. First pass metabolism occurs in the liver, thus inhibitors of the corresponding catabolic enzymes may enhance the effect of the cognate drugs. Reversible inhibitors would be delivered at the same time as, or slightly before, the drug of interest. Screening of libraries in hepatocytes for inhibitors (by any mechanism, such as protein downregulation as well as a direct inhibition of activity) of particularly problematical isozymes would be of interest. These include the CYP3A4 isozymes of cytochrome P450, which are involved in the first pass metabolism of the anti-HIV drugs saquinavir and indinavir. Other applications could include reversible inhibitors of UDP-glucuronyltransferases, sulfotransferases, N-acetyltranferases, epoxide hydrolases, and glutathione S-transferases, depending on the drug. Screens would be done in cultured hepatocytes or liver microsomes, and involve antibodies recognizing the specific modification performed in the liver, or cocultured readout cells, if the metabolite had a different bioactivity than the untransformed drug.

In a preferred embodiment, the present methods are useful in immunobiology, inflammation, and allergic response applications. Selective regulation of T lymphocyte responses is a desired goal in order to modulate immune-mediated diseases in a specific manner. Libraries can be introduced into specific T cell subsets (TH1, TH2, CD4+, CD8+, and others) and the responses which characterize those subsets (cytokine generation, cytotoxicity, proliferation in response to antigen being presented by a mononuclear leukocyte, and others) are modified by members of the library. Intracellular immunoglobulin molecules, or fragments thereof can be selected which increase or diminish the known Tcell subset physiologic response. This approach will be useful in any number of conditions, including: 1) autoimmune diseases where one wants to induce a tolerant state (select an intracellular immunoglobulin molecule or fragment thereof that inhibits T cell subset from recognizing a self-antigen bearing cell); 2) allergic diseases where one wants to decrease the stimulation of IgE producing cells (select intracellular immunoglobulin molecules, or fragments thereof which block release from T cell subsets of specific B-cell stimulating cytokines which induce switch to IgE production); 3) in transplant patients where one wants to induce selective immunosuppression (select peptide that diminishes proliferative responses of host T cells to foreign antigens); 4) in lymphoproliferative states where one wants to inhibit the growth or sensitize a specific T cell tumor to chemotherapy and/or radiation; 5) in tumor surveillance, where one wants to inhibit the killing of cytotoxic T cells by Fas ligand bearing tumor cells; and 5) in T cell mediated inflammatory diseases such as Rheumatoid arthritis, connective tissue diseases (SLE), multiple sclerosis, and inflammatory bowel disease, where one wants to inhibit the proliferation of disease-causing T cells (promote their selective apoptosis) and the resulting selective destruction of target tissues (cartilage, connective tissue, oligodendrocytes, gut endothelial cells, respectively).

Regulation of B cell responses will permit a more selective modulation of the type and amount of immunoglobulin made and secreted by specific B cell subsets. Libraries can be introduced into B cells and intracellular immunoglobulin molecules, or fragments thereof selected which inhibit the release and synthesis of a specific immunoglobulin. This may be useful in autoimmune diseases characterized by the overproduction of auto antibodies and the production of allergy causing antibodies, such as IgE. Intracellular immunoglobulin molecules, or fragments thereof can also be identified which inhibit or enhance the binding of a specific immunoglobulin subclass to specific antigen either foreign or self. Finally, intracellular immunoglobulin molecules, or fragments thereof can be selected which inhibit the binding of a specific immunoglobulin subclass to its receptor on specific cell types.

Similarly, intracellular immunoglobulin molecules, or fragments thereof which affect cytokine production may be selected, generally using two cell systems. For example, cytokine production from macrophages, monocytes, etc. may be evaluated. Similarly, intracellular immunoglobulin molecules, or fragments thereof which enhance cytosine responses, may be selected.

Antigen processing by mononuclear leukocytes (ML) is an important early step in the immune system's ability to recognize and eliminate foreign proteins. Candidate intracellular immunoglobulin molecules, or fragments thereof can be introduced into ML cell lines and intracellular immunoglobulin molecules, or fragments thereof selected which alter the intracellular processing of foreign peptides and sequence of the foreign peptide that is presented to T cells by MLs on their cell surface in the context of class II MHC. One can look for members of the library that enhance immune responses of a particular T cell subset (for example, the intracellular immunoglobulin molecules, or fragments thereof would in fact work as a vaccine), or look for a library member that binds more tightly to MHC, thus displacing, naturally occurring processed peptides, thus the intracellular immunoglobulin molecule or fragment thereof would be less immunogenic (less stimulatory to a specific T cell clone). These intracellular immunoglobulin molecules, or fragments thereof would in fact induce immune tolerance and/or diminish immune responses to foreign proteins. This approach could be used in transplantation, autoimmune diseases, and allergic diseases.

The release of inflammatory mediators (cytokines, leukotrienes, prostaglandins, platelet activating factor, histamine, neuropeptides, and other peptide and lipid mediators) is a key element in maintaining and amplifying aberrant immune responses. Libraries can be introduced into MLs, mast cells, eosinophils, and other cells participating in a specific inflammatory response, and intracellular immunoglobulin molecules, or fragments thereof selected which inhibit the synthesis or release of each of these types of mediators.

In a preferred embodiment, the present methods are useful in biotechnology applications. Candidate library expression in mammalian cells can also be considered for other phamaceutical-related applications, such as modification of protein expression, protein folding, or protein secretion. One such example would be in commercial production of protein pharmaceuticals in CHO or other cells. Libraries resulting in intracellular immunoglobulin molecules, or fragments thereof which select for an increased cell growth rate (perhaps intracellular immunoglobulin molecules, or fragments thereof mimicking growth factors or acting as agonists of growth factor signal transduction pathways), for pathogen resistance (see previous section), for lack of sialylation or glycosylation (by blocking glycotransferases or rerouting trafficking of the protein in the cell), for allowing growth on autoclaved media, or for growth in serum free media, would all increase productivity and decrease costs in the production of protein pharmaceuticals.

Igs and Ig fragments can be used as tools to identify organ, tissue, and cell specific cell surface antigens by screening for the loss of expression of a receptor or epitope. The Igs or Ig fragment identified can then be coupled to an enzyme, drug, imaging agent or substance for which organ targeting is desired.

Other intracellular immunoglobulin molecules, or fragments thereof which may be selected using the present invention include: 1) intracellular immunoglobulin molecules, or fragments thereof which block the activity of transcription factors, using cell lines with reporter genes; 2) intracellular immunoglobulin molecules, or fragments thereof which block the interaction of two known proteins in cells, using the absence of normal cellular functions, the mammalian two hybrid system or fluorescence resonance energy transfer mechanisms for detection; and 3) intracellular immunoglobulin molecules, or fragments thereof may be identified by tethering a Igs or Ig fragment to a protein binding region to allow interactions with molecules sterically close, e.g., within a signalling pathway to localize the effects in a functional area of interest.

Additionally, intracellular immunoglobulin molecules, or fragments thereof that modulate infectivity of infectious agents may be screened for or selected. Examples of such infectious agents include, but are not limited to, bacteria, viral, parasite, and fungal. Examples of viral agents include, but are not limited to, adenovirus, alphavirus, calicivirus, coronavirus, distemper virus, Ebola virus, enterovirus, flavivirus, hepatitis virus (A-E), herpesvirus, immunodeficiency virus, infectious peritonitis virus, influenza virus, leukemia virus, Marburg virus, oncogenic virus, orthomyxovirus, papilloma virus, parainfluenza virus, paramyxovirus, parvovirus, pestivirus, picoma virus, rabies virus, reovirus, retrovirus, rotavirus, as well as other cancer-causing or cancer-related viruses.

Examples of bacterial agents include, but are not limited to, Actinomyces, Bacillus, Bacteroides, Bordetella, Bartonella, Borrelia, Brucella, Campylobacter, Capnocytophaga, Chlamydia, Clostridium, Corynebacterium, Coxiella, Dermatophilus, Enterococcus, Ehrlichia, Escherichia, Francisella, Fusobacterium, Haemobartonella, Haemophilus, Helicobacter, Klebsiella, L-form bacteria, Leptospira, Listeria, Mycobacteria, Mycoplasma, Neisseria, Neorickettsia, Nocardia, Pasteurella, Peptococcus, Peptostreptococcus, Pneumococcus, Proteus, Pseudomonas, Rickettsia, Rochalimaea, Salmonella, Shigella, Staphylococcus, Streptococcus, Treponema, and Yersinia.

Examples of fungal agents include, but are not limited to, Absidia, Acremonium, Alternaria, Aspergillus, Basidiobolus, Bipolaris, Blastomyces, Candida, Coccidioides, Conidiobolus, Cryptococcus, Curvalaria, Epidermophyton, Exophiala, Geotrichum, Histoplasma, Madurella, Malassezia, Microsporum, Moniliella, Mortierella, Mucor, Paecilomyces, Penicillium, Phialemonium, Phialophora, Prototheca, Pseudallescheria, Pseudomicrodochium, Pythium, Rhinosporidium, Rhizopus, Scolecobasidium, Sporothrix, Stemphylium, Trichophyton, Trichosporon, and Xylohypha.

Examples of protozoan parasites include, but are not limited to, Babesia, Balantidium, Besnoitia, Cryptosporidium, Eimeri a, Encephalitozoon, Entamoeba, Giardia, Hammondia, Hepatozoon, Isospora, Leishmania, Microsporidia, Neospora, Nosema, Pentatrichomonas, Plasmodium, Pneumocystis, Sarcocystis, Schistosoma, Theileria, Toxoplasma, and Trypanosoma.

Examples of helminth parasites include, but are not limited to, Acanthocheilonema, Aelurostrongylus, Ancylostoma, Angiostrongylus, Ascaris, Brugia, Bunostomum, Capillaria, Chabertia, Cooperia, Crenosoma, Dictyocaulus, Dioctophyme, Dipetalonema, Diphyllobothrium, Diplydium, Dirofilaria, Dracunculus, Enterobius, Filaroides, Haemonchus, Lagochilascaris, Loa, Mansonella, Muellerius, Nanophyetus, Necator, Nematodirus, Oesophagostomum, Onchocerca, Opisthorchis, Ostertagia, Parafilaria, Paragonimus, Parascaris, Physaloptera, Protostrongylus, Setaria, Spirocerca,antigens Spirometra, Stephanofilaria, Strongyloides, Strongylus, Thelazia, Toxascaris, Toxocara, Trichinella, Trichostrongylus, Trichuris. Uncinaria, and Wuchereria.

Kits. The present invention further provides a kit for the selection of intracellular immunoglobulin molecules, or fragments thereof expressed in a eukaryotic host cell. The kit comprises one or more containers filled with one or more of the ingredients required to carry out the methods described herein.

In one embodiment, the kit comprises: (a) a first library of polynucleotides encoding, through operable association with a transcriptional control region, a plurality of first intracellular immunoglobulin subunit polypeptides, where each first intracellular immunoglobulin subunit polypeptide comprises a first immunoglobulin variable region selected from the group consisting of a heavy chain variable region and a light chain variable region, wherein said first library is constructed in a eukaryotic virus vector; (b) a second library of polynucleotides encoding, through operable association with a transcriptional control region, a plurality of second intracellular immunoglobulin subunit polypeptides, where each comprises: a second immunoglobulin variable region selected from the group consisting of a heavy chain variable region and a light chain variable region, wherein said second immunoglobulin variable region is not the same as the first immunoglobulin variable region, where the second intracellular immunoglobulin subunit polypeptide is capable of combining with the first intracellular immunoglobulin subunit polypeptide to form a immunoglobulin molecule, or fragment thereof, and where the second library is constructed in a eukaryotic virus vector; and (c) a population of host cells capable of expressing said immunoglobulin molecules. In this kit, the first and second libraries are provided both as infectious virus particles and as inactivated virus particles, where the inactivated virus particles are capable of infecting the host cells and allowing expression of the polynucleotides contained therein, but the inactivated viruses do not undergo virus replication.

Alternatively, the kit comprises a single immunoglobulin library encoding a single-chain intracellular immunoglobulins, as described herein.

In addition, the host cells provided with the kit are capable of expressing an immunoglobulin molecule. Use of the kit is in accordance to the methods described herein. In certain embodiments the kit will include control antigens and reagents to standardize the validate the selection of particular antigens of interest.

Isolated immunoglobulins. The present invention further provides an isolated intracellular immunoglobulin or fragment thereof produced by any of the methods disclosed herein. Such isolated immunoglobulins may be useful as diagnostic or therapeutic reagents. Further provided is a composition comprising an isolated intracellular immunoglobulin of the present invention, and a pharmaceutically acceptable carrier.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., Sambrook et al., ed., Cold Spring Harbor Laboratory Press: (1989); Molecular Cloning: A Laboratory Manual, Sambrook et al., ed., Cold Springs Harbor Laboratory, New York (1992), DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); and in Ausubel et al., Current Protocols in Molecular Biology, John Wiley and Sons, Baltimore, Md. (1989).

General principles of antibody engineering are set forth in Antibody Engineering, 2nd edition, C. A. K. Borrebaeck, Ed., Oxford Univ. Press (1995). General principles of protein engineering are set forth in Protein Engineering, A Practical Approach, Rickwood, D., et al., Eds., IRL Press at Oxford Univ. Press, Oxford, Eng. (1995). General principles of antibodies and antibody-hapten binding are set forth in: Nisonoff, A., Molecular Immunology, 2nd ed., Sinauer Associates, Sunderland, Mass. (1984); and Steward, M. W., Antibodies, Their Structure and Function, Chapman and Hall, New York, N.Y. (1984). Additionally, standard methods in immunology known in the art and not specifically described are generally followed as in Current Protocols in Immunology, John Wiley & Sons, New York; Stites et al. (eds), Basic and Clinical-Immunology (8th ed.), Appleton & Lange, Norwalk, Conn. (1994) and Mishell and Shiigi (eds), Selected Methods in Cellular Immunology, W. H. Freeman and Co., New York (1980).

Standard reference works setting forth general principles of immunology include Current Protocols in Immunology, John Wiley & Sons, New York; Klein, J., Immunology: The Science of Self-Nonself Discrimination, John Wiley & Sons, New York (1982); Kennett, R., et al., eds., Monoclonal Antibodies, Hybridoma: A New Dimension in Biological Analyses, Plenum Press, New York (1980); Campbell, A., “Monoclonal Antibody Technology” in Burden, R., et al., eds., Laboratory Techniques in Biochemistry and Molecular Biology, Vol. 13, Elsevere, Amsterdam (1984).

### EXAMPLES

### EXAMPLE 1

### Construction of Human Intrabody Libraries of Diverse Specificity

Libraries of polynucleotides encoding fully human, diverse intrabodies are produced as follows. These are Fab fragments which comprise a heavy chain variable region linked to a first constant region domain (VH-CH1) paired with an immunoglobulin light chain. Genes for human VH (variable region of heavy chain), VK (variable region of kappa light chain) and VL (variable region of lambda light chains) are amplified by PCR. For each of the three variable gene families, both a recombinant plasmid library and a vaccinia virus library is constructed. The variable region genes are inserted into a p7.5/tk-based transfer/expression plasmid immediately upstream of a constant region sequence corresponding to the CH1 domain of heavy chains or the kappa light chain constant region, CK. These plasmids are employed to generate the corresponding vaccinia virus recombinants by trimolecular recombination and can also be used directly for high level expression of Fab fragments following transfection of one immnunoglobulin chain orfragment thereof into cells infected with vaccinia virus recombinants of a second immunoglobulin chain or fragment thereof. The two chains are synthesized and assembled to form an Fab fragment. These Fab fragments may be localized to different cellular compartments and organelles by attachin coding sequences for subcellular localization signals.

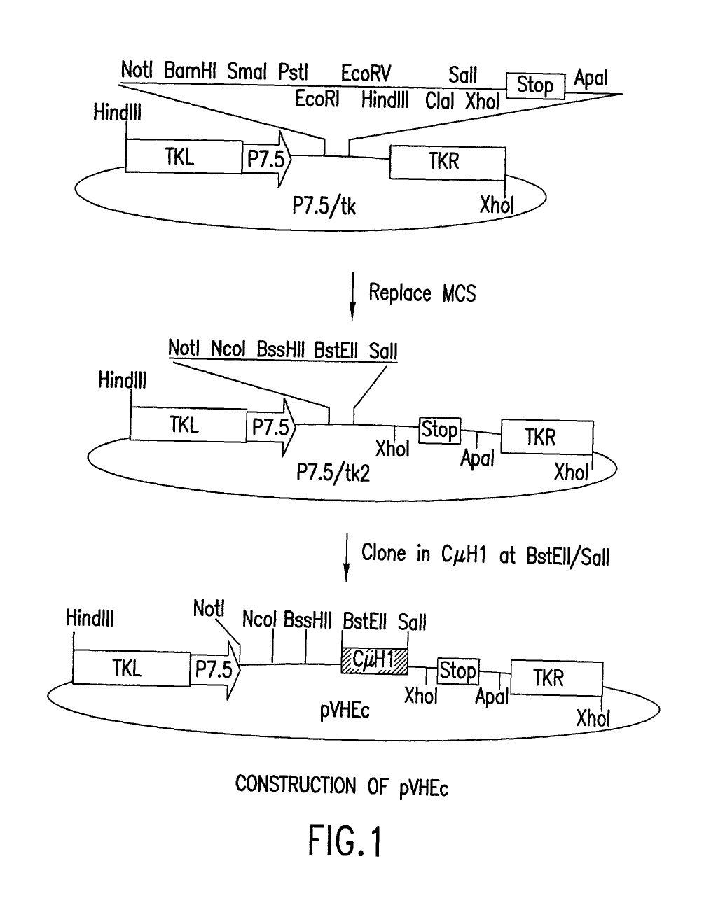

1.1 pVHEc. An expression vector which encodes a human heavy chain fragment comprising VH and the CH1 domain of Cμ, designated pVHEc, is constructed as follows, as illustrated in FIG. 1. Plasmid p7.5/tk, produced as described in Zauderer, PCT Publication No. WO 00/028016 is converted into p7.5/tk2 by the following method. The multiple cloning site (MCS) of p7.5/tk is replaced with a cassette containing the following restriction sites: NotI-NcoI-BssHII-PstI-BstEII-SalI to generate p7.5/tk2. This cassette, having the sequence 5′-GCGGCCGCAA ACCATGGAAA GCGCGCATAT GGTCACCAAA AGTCGAC-3′,is referred to herein as SEQ ID NO:78. A cDNA coding for the human IgM heavy chain is isolated from bone marrow RNA using SMART™ RACE cDNA Amplification Kit available from Clontech, Palo Alto, Calif., using standard methods. A DNA construct encoding amino acids 109-113 of VH and the CH1 domain, i.e., amino acids 109-223B of Cμ, is amplified from the isolated IgM heavy chain cDNA, using primers that include a BstEII site at the 5′ end of the region encoding amino acids 109-113+ the Cμ CH1 domain, and a stop codon and a SalI site at its 3′ end. These primers have the following sequences: huCμ5: 5′-ATTAGGTCAC CGTCTCCTCA GGG-3′ (SEQ ID NO:79); and huCμ3:5′-ATTAGTCGACTCATGGAAGA GGCACGTTCTT-3′ (SEQ ID NO:80). The digested PCR product is inserted into p7.5/tk2 between the BstEII and SalI sites to generate pVHEc. Heavy chain variable region (VH) PCR products (amino acids (−4) to (110)), produced as described in Example 1.3(a), herein, using the primers listed in Table 3, are cloned into the BssHII and BstEII sites of pVHEc. Because of the overlap between the CH1 domain sequence and the restriction enzyme sites selected, this results in construction of a contiguous heavy chain fragment which lacks a functional signal peptide but remains in the correct translational reading frame.

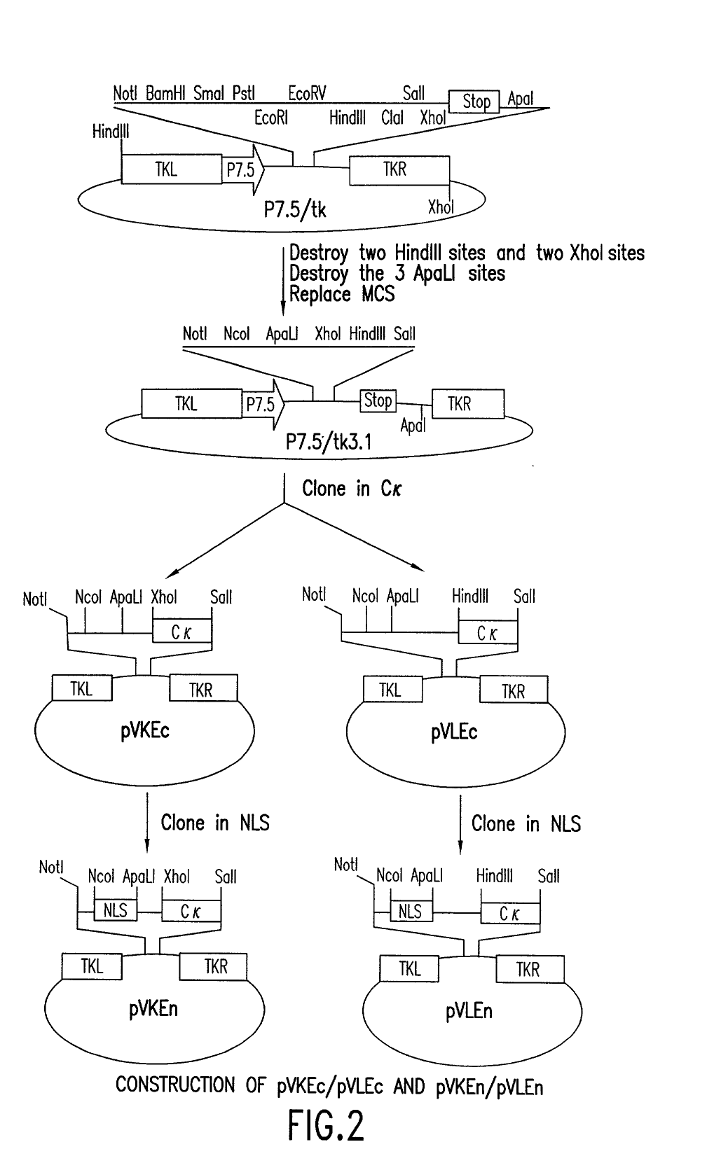

1.2 pVKEc and pVLEc. Expression vectors encoding the human κ and λ immunoglobulin light chain constant regions, designated herein as pVKEc and pVLEc, are constructed as follows, as illustrated in FIG. 2.

(a) Plasmid p7.5/tk is converted into p7.5/tk3.1 by the following method. The two XhoI sites and two HindIII sites of p7.5/tk are removed by fill-in ligation, the 3 ApaLI sites (one at the backbone, one at ColE1 ori, and the other at Amp) are removed by standard methods, and the multiple cloning site (MCS) of p7.5/tk is replaced with a cassette containing the following restriction sites: NotI-NcoI-ApaLI-XhoI-HindIII-SalI to generate p7.5/tk3. 1. This cassette, having the sequence 5′-GCGGCCGCCC ATGGATAGCG TGCACTTGAC TCGAGAAGCT TAGTAGTCGA C-3′, is referred to herein as SEQ ID NO:81.

(b) Plasmid p7.5/tk3.1 is converted into pVKEc by the following method. A cDNA coding for the Cκ region is isolated from bone marrow RNA using SMART™ RACE cDNA Amplification Kit as described above, with primers to include an XhoI site at the 5′ end of the region encoding amino acids 104-107+Cκ, a stop codon, and a SalI site at its 3′ end. These primers have the following sequences: huCκ5:5′-CACGACTCGA GATCAAACGA ACTGTGGCTG-3′ (SEQ ID NO:82); and huCκ3:5′-AATATGTCGA CCTAACACTC TCCCCTGTTG AAGCTCTT-3′ (SEQ ID NO:83). The digested cDNA fragment is then cloned into p7.5/tk3.1 at XhoI and SalI sites to generate pVKEc. Kappa light chain variable region (VK) PCR products (amino acids (−3) to (105)), produced as described in Example 1.3(b), herein, using the primers listed in Table 3, are then cloned into pVKEc at the ApaLI and XhoI sites. Because of the overlap between the κ light chain sequence and the restriction enzyme sites selected, this results in construction of contiguous κ light chains which lacks a functional signal peptide but remains in the correct translational reading frame.

(c) Plasmid p7.5/tk3.1 is converted into pVLEc by the following method. A cDNA coding for the Cκ region is isolated from bone marrow RNA using SMART™ RACE cDNA Amplification Kit as described above, with primers to include a HindIII site and the region encoding amino acids 105 to 107 of V_{λ} at its 5′ end and a stop codon and a SalI site at its 3′ end. These primers have the following sequences: huCλ5:5′-ATTTAAGCTT ACCGTCCTAC GAACTGTGGC TGCACCATCT-3′ (SEQ ID NO:84); and huCλ3 (SEQ ID NO:83). The digested PCR product is then cloned into p7.5/tk3.1 at HindIII and SalI sites to generate pVLEc. Lambda light chain variable region (VL) PCR products (amino acids (−3) to(104)), produced as described in Example 1.3(c), herein, using the primers listed in Table 3, are then cloned into pVLEc at ApaLI and HindIII sites. Because of the overlap between the λ light chain sequence and the restriction enzyme sites selected, this results in construction of contiguous λ light chains which lacks a functional signal peptide but remains in the correct translational reading frame.

1.3 Variable Regions. Heavy chain, kappa light chain, and lambda light chain variable regions are isolated by PCR for cloning in the expression vectors produced as described above, by the following method. RNA isolated from normal human bone marrow pooled from multiple donors (available from Clontech) is used for cDNA synthesis. Aliquots of the cDNA preparations are used in PCR amplifications with primer pairs selected from the following sets of primers: VH/JH, VK/JK or VL/JL. The primers used to amplify variable regions are listed in Tables 1 and 2.

(a) Heavy chain variable regions. Due to the way the plasmid expression vectors were designed, VH primers, i.e., the forward primer in the pairs used to amplify heavy chain V regions, have the following generic configuration, with the BssHII restriction site in bold:

VH primers: GCGCGCACTCC-start of VH FR1 primer (SEQ ID NO:154).

The primers are designed to include codons encoding the last 4 amino acids in the leader, with the BssHII site coding for amino acids −4 and −3, followed by the VH family-specific FR1 sequence. Tables 1 and 2 lists the sequences of the different family-specific VH primers. Since the last 5 amino acids of the heavy chain variable region, i.e., amino acids 109-113, which are identical among the six human heavy chain J regions, are embedded in plasmid pVHE, JH primers, i.e., the reverse primers used to amplify the heavy chain variable regions, exhibit the following configuration to include a BstEII site, which codes for amino acids 109 and 110 (shown in bold):

JH primers:

-nucleotide sequence for amino acids 103-108 of VH (ending with a G)-GTCACC

Using these sets of primers, the VH PCR products start with the codons coding for amino acids −4 to 110 with BssHII being amino acids −4 and −3, and end at the BstEII site at the codons for amino acids 109 and 110 . Upon digestion with the appropriate restriction enzymes, these PCR products are cloned into pVHE digested with BssHII and BstEII.

In order to achieve amplification of most of the possible rearranged heavy chain variable regions, families of VH and JH primers, as shown in Tables 1 and 2, are used. The VH1, 3, and 4 families account for 44 out of the 51 V regions present in the human genome. The embedding of codons coding for amino acids 109-113 in the expression vector precludes the use of a single common JH primer. However, the 5 JH primers shown in Tables 1 and 2 can be pooled for each VH primer used to reduce the number of PCR reactions required.

(b) Kappa light chain variable regions. The VK primers, i.e., the forward primer in the pairs used to amplify kappa light chain variable regions, have the following generic configuration, with the ApaLI restriction site in bold:

VK primer: GTGCACTCC-start of VK FR1 primer

The VK primers contain codons coding for the last 3 amino acids of the kappa light chain leader with the ApaLI site coding for amino acids −3 and −2, followed by the VK family-specific FRI sequences. Since the codons encoding the last 4 amino acids of the kappa chain variable region (amino acids 104-107) are embedded in the expression vector pVKE, the JK primers, i.e., the reverse primer in the pairs used to amplify kappa light chain variable regions, exhibit the following configuration:

JK primer:

-nucleotide sequence coding for amino acids 98-103 of VK-CTCGAG

The XhoI site (shown in bold) comprises the codons coding for amino acids 104-105 of the kappa light chain variable region. The PCR products encoding kappa light chain variable regions start at the codon for amino acid −3 and end at the codon for amino acid 105, with the ApaLI site comprising the codons for amino acids −3 and −2 and the XhoI site comprising the codons for amino acids 104 and 105. VK1/4 and VK3/6 primers each have two degenerate nucleotide positions. Employing these JK primers (see Tables 1 and 2), JK1, 3 and 4 will have a Val to Leu mutation at amino acid 104, and JK3 will have an Asp to Glu mutation at amino acid 105.

(c) Lambda light chain variable regions. The VL primers, i.e., the forward primer in the pairs used to amplify lambda light chain variable regions, have the following generic configuration, with the ApaLI restriction site in bold:

VL primer: GTGCACTCC-start of VL

The ApaLI site comprises the codons for amino acids −3 and −2, followed by the VL family-specific FR1 sequences. Since the codons encoding the last 5 amino acids of VL (amino acids 103-107) are embedded in the expression vector pVLE, the JL primers exhibit the following configuration to include a HindIII site (shown in bold) comprising the codons encoding amino acids 103-104:

JL primer: -nucleotide sequence for amino acids 97-102 of VL-AAGCTT

The PCR products encoding lambda light chain variable regions start at the codon for amino acid −3 and end at the codon for amino acid 104 with the ApaLI site comprising the codons for amino acids −3 and −2, and HindIII site comprising the codons for amino acids 103 and 104.

TABLE 3
Oligonucleotide primers for PCR amplification
of human immunoglobulin variable regions.
Recognition sites for restriction enzymes used
in cloning are indicated in bold type.
Primer sequences are from 5′ to 3′.

| | | |
|---|---|---|
| VH1 | (SEQ ID NO:85) | AATA TGC GCG CAC TCC CAG |
| | | GTG CAG CTG GTG CAG TCT GG |
| VH2 | (SEQ ID NO:86) | AATA TGC GCG CAC TCC CAG |
| | | GTC ACC TTG AAG GAG TCT GG |
| VH3 | (SEQ ID NO:87) | AATA TGC GCG CAC TCC GAG |
| | | GTG CAG CTG GTG GAG TCT GG |
| V114 | (SEQ ID NO:88) | AATA TGC GCG CAC TCC CAG |
| | | GTG CAG CTG GAG GAG TCG GG |
| VH5 | (SEQ ID NO:89) | AATA TGC GCG CAC TCC GAG |
| | | GTG CAG CTG GTG GAG TGT G |
| JH1 | (SEQ ID NO:90) | GA GAG GGT GAC CAG GGT GCC |
| | | CTG GCC CCA |
| JH2 | (SEQ ID NO:91) | GA GAG GGT GAC CAG GGT GCC |
| | | ACG GCC CCA |
| JH3 | (SEQ ID NO:92) | GA GAC GGT GAC CAT TGT CCC |
| | | TTG GCC CCA |
| JH4/5 | (SEQ ID NO:93) | GA GAG GGT GAC CAG GGT TCC |
| | | CTG GCC CCA |
| JH6 | (SEQ ID NO:94) | GA GAC GGT GAC CGT GGT CCC |
| | | TTG GCC CCA |
| VK1 | (SEQ ID NO:95) | CAGGA GTG CAC TCC GAG ATC |
| | | CAG ATG ACC GAG TCT CC |
| VK2 | (SEQ ID NO:96) | CAGGA GTG CAC TCC GAT GTT |
| | | GTG ATG ACT CAG TCT CC |
| VK3 | (SEQ ID NO:97) | CAGGA GTG CAC TCC GAA ATT |
| | | GTG TTG ACG CAG TCT CC |
| VK4 | (SEQ ID NO:98) | CAGGA GTG CAC TCC GAC ATC |
| | | GTG ATG ACC CAG TCT CC |
| VK5 | (SEQ ID NO:99) | CAGGA GTG CAC TCC GAA ACG |
| | | ACA CTC ACG CAG TCT CC |
| VK6 | (SEQ ID NO:100) | CAGGA GTG CAC TCC GAA ATT |
| | | GTG CTG ACT CAG TCT CC |
| JK1 | (SEQ ID NO:101) | TT GAT CTC GAG CTT GGT CCC |
| | | TTG GCC GAA |
| JK2 | (SEQ ID NO:102) | TT GAT CTC GAG CTT GGT CCC |
| | | CTG GCC AAA |
| JK3 | (SEQ ID NO:103) | TT GAT CTC GAG TTT GGT CCC |
| | | AGG GCC GAA |
| JK4 | (SEQ ID NO:104) | TT GAT CTC GAG CTT GGT CCC |
| | | TCC GCC GAA |
| JK5 | (SEQ ID NO:105) | TT AAT CTC GAG TCG TGT CCC |
| | | TTG GCC GAA |
| VL1 | (SEQ ID NO:106) | CAGAT GTG CAC TCC CAG TCT |
| | | GTG TTG ACG CAG CCG CC |
| VL2 | (SEQ ID NO:107) | CAGAT GTG CAC TCC CAG TCT |
| | | GCC CTG ACT CAG CCT GC |
| VL3A | (SEQ ID NO:108) | CAGAT GTG CAC TCC TCC TAT |
| | | GTG CTG ACT CAG CCA CC |
| VL3B | (SEQ ID NO:109) | CAGAT GTG CAC TCC TCT TCT |
| | | GAG CTG ACT GAG GAC CC |
| VL4 | (SEQ ID NO:110) | CAGAT GTG CAC TCC CAC GTT |
| | | ATA CTG ACT CAA CCG CC |
| VL5 | (SEQ ID NO:111) | CAGAT GTG CAC TCC CAG GCT |
| | | GTG CTC ACT CAG CCG TC |
| VL6 | (SEQ ID NO:112) | CAGAT GTG CAC TCC AAT TTT |
| | | ATG CTG ACT GAG CCC CA |
| VL7 | (SEQ ID NO:113) | CAGAT GTG CAC TCC CAG GCT |
| | | GTG GTG ACT CAG GAG CC |
| JL1 | (SEQ ID NO:114) | AC GGT AAG CTT GGT CCC AGT |
| | | TCC GAA GAC |
| JL2/3 | (SEQ ID NO:115) | AC GGT AAG CTT GGT CCC TCC |
| | | GCC GAA TAC |

1.4 Expression of Fab in other organelles. The cytoplasmic expression vectors (pVHEc, pVKEc and pVLEc) serve as the prototype vectors into which other organelle-specific localization signals or combinations thereof can be cloned to target Fab to specific subcellular compartments. Examples of localization signals are shown in Table 4. To target Fab to the endoplasmic reticulum (ER), both a signal peptide at the amino terminus and an ER retention signal (KDEL) at the C-terminus are required. To target Fab to the nucleus, a nuclear localization signal (PKKKRKV) is appended to the N-terminus. Fab molecules can also be anchored to the inner leaflet of the plasma membrane through the addition of myristylation signal at the N-terminus or palmitoylation or prenylation signal at the C-terminus. To target Fab to lysosomes or mitochondria, a lysosomal or mitochondrial targeting sequence is added to the amino-terminus. These localization signals may be inserted either in the N-terminus of Fab between NcoI and BssHII of pVHEc, between NcoI and ApaLI of pVKEc and pVLEc, and/or in the C-terminus at SalI site.

TABLE 4
Localization Signals

| | | | | | SEQ ID |
|---|---|---|---|---|---|
| Localization sequence | Terminus | Location | Protein | Ref | NO: |
| MGWSCIILFLVATATGAHS | N | ES | IgG1 | 1 | 116 |
| NLWTTASTFIVLFLLSLFYS | C/N | PM | IgM | 2 | 117 |
| TTVTLF | | | | | |
| KDEL | C | ER | calreticulin | 3 | 118 |
| PKKKRKV | N | N | LargeT | 4 | 119 |
| MGSSKSKPKDPSQR | N | PMi | c-src | 5 | 120 |
| LNPPDESGPGCMSCKCVLS | C | PMi | H-ras1 | 6 | 121 |
| KFERQ | N | L | Lamp-2 | 7 | 122 |
| MSVLTPLLLRGLTGSARRL | N | M | CoxVIII | 8 | 123 |
| PVPRAKIHSL | | | | | |

Abbreviations for items under Location: ES, extracellular space; PM, plasma membrane; ER, endoplasmic reticulum; N, nucleus; PMi, inner leaflet of plasma membrane; L, lysosome; M, mitochondrion.
References:
1. Persic et al., Gene 187: 1 (1997)
2. Friedlander et al., Nucleic Acids Res. 18: 4278 (1990)
3. Munro et al., Cell 48: 899 (1987)
4. Lanford et al., Cell 46: 575 (1986)
5. Cross et al., Mol. Cell. Biol. 4: 1834 (1984)
6. Capon et al., Nature 302: 33 (1983)
7. Dice, Ann. N. Y. Acad. Sci. 674: 58 (1992)
8. Rizzuto et al., Nature 358: 325 (1992)

### EXAMPLE 2

### Vectors For Expression of Fab in The Mammalian Two-hybrid System

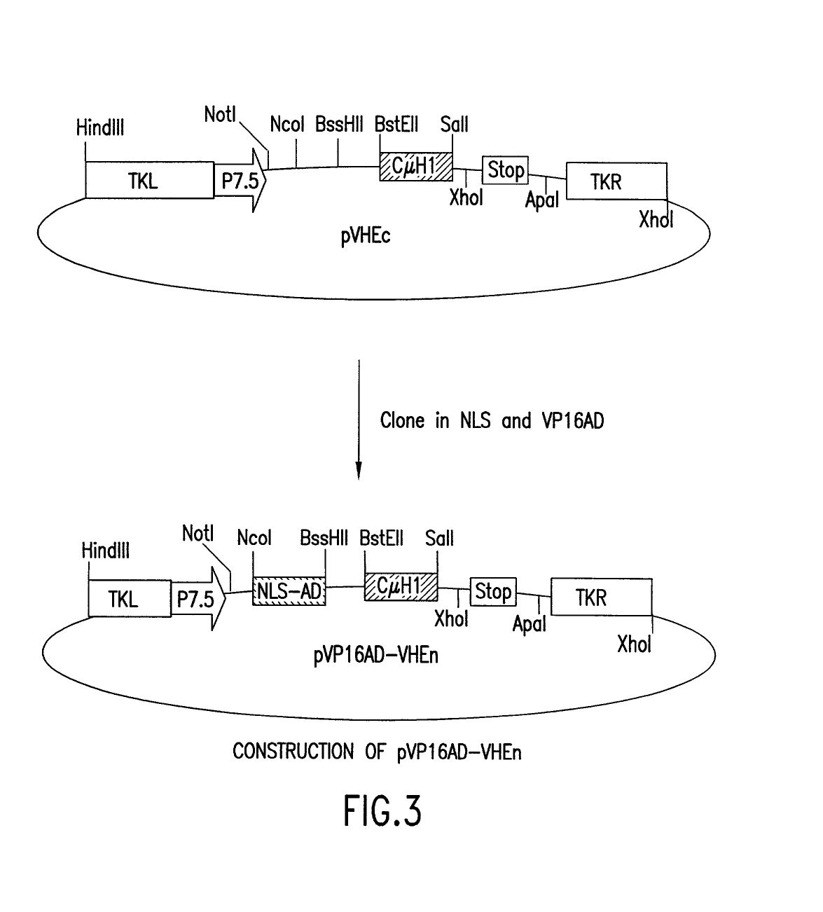

2.1 Construction of Fab expression vectors. (a) Plasmid pVP16AD-VHEn, a VH-CH1 expression vector comprising a nuclear localization signal and the activation domain of VP16, is produced by the following method, as illustrated in FIG. 3. Plasmid pVHEc is prepared as described in Example 1.1. Cassettes encoding the SV40 large T-antigen nuclear localization signal (Table 4) and the activation domain of VP16 are amplified by PCR from vector pVP16 (Clontech). The primers are designed to add 5′ NcoI and 3′ BssHII sites for subcloning into pVHEc at NcoI/BssHII sites. These primers have the following sequences: forward primer:5′-GCCACCATGG GCCCTAAAAA GAAG-3′ (SEQ ID NO:124); and reverse primer: 5′-ATTAGCGCGC TCCCACCGTA CTCGTCAAT-3′ (SEQ ID NO:125). VH genes are PCR amplified as described in Example 1.3, using the primers listed in Table 3, and PCR products are subcloned into pVP16AD-VHEn at BssHII/BstEII sites as described above.

(b) Plasmids pVKEn and pVLEn, kappa and lambda light chain expression vectors comprising a nuclear localization signal, are produced by the following method, as illustrated in FIG. 2. Plasmids pVKEc and pVLEc are produced as described in Example 1.2. A nucleotide cassette encoding a nuclear localization signal, for example, the SV40 large T NLS listed in Table 4, flanked by NcoI and ApaLI sites is inserted at NcoI/ApaLI sites of pVKEc to generate pVKEn and at NcoI/ApaLI sites of pVLEc to generate pVLEn. VK and VL genes are PCR amplified by the methods described in Example 1.3, using the primers listed in Table 3. The variable region PCR products are subcloned into pVKEn at ApaLI/XhoI sites and into pVLEn at ApaLI/HindIII sites as described above.

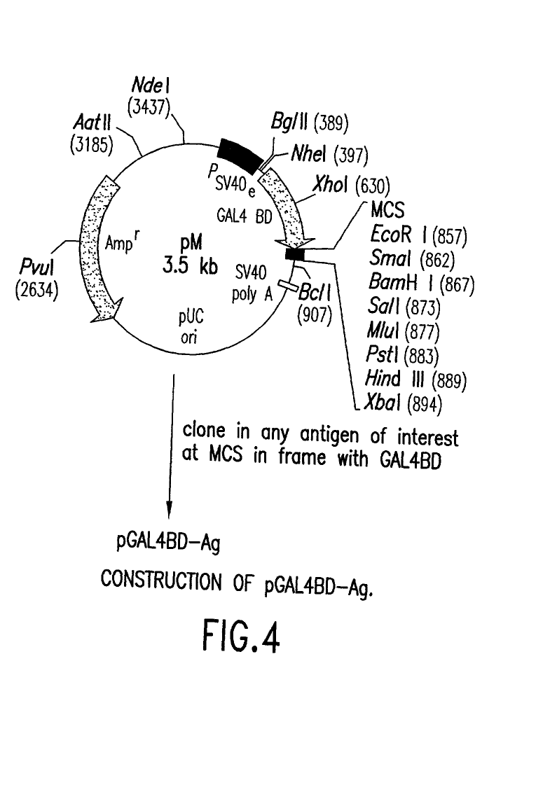

2.2 Construction of pGAL4BD-Ag vectors. Vectors for expression GAL4 binding domain-Ag fusions proteins are prepared by the following method, as illustrated in FIG. 4. The pM vector (Clontech) is employed as the parental vector. This vector contains the gene coding for the DNA binding domain of GAL4 under the control of the SV40 early promoter. The introduction of a gene encoding an antigen of interest in frame in the MCS in this vector results in the production of a GAL4BD-Ag fusion protein.

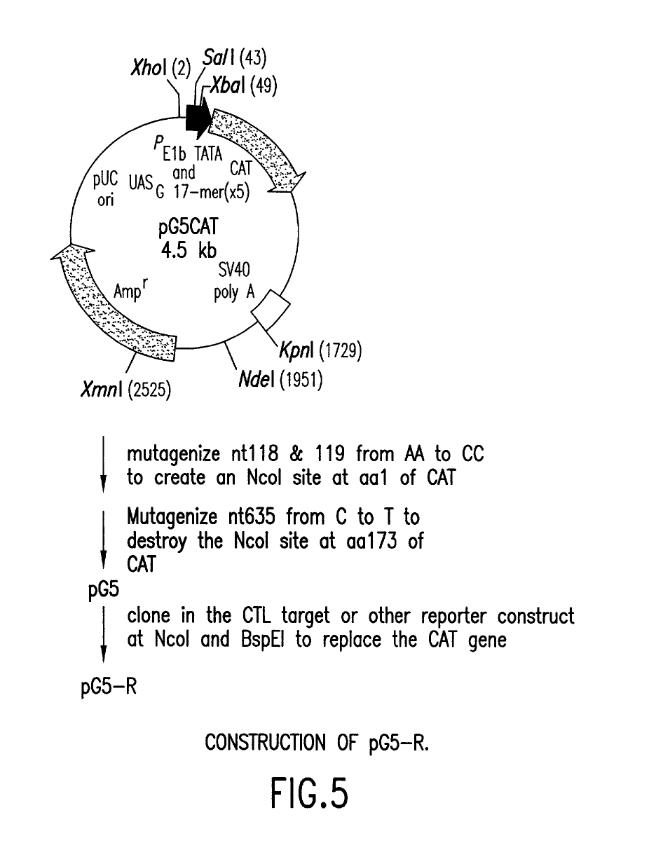

2.3 Construction of pG5-R reporter vectors (pG5-R). Reporter constructs to be expressed under the control of GAL4 are produced by the following method, as illustrated in FIG. 5. Plasmid pG5 CAT (Clontech) contains five consensus GAL4 binding sites and an adenovirus E1b minimal promoter upstream of a reporter gene encoding chloramphenicol acetyl transferase (CAT). It is used as the parental vector to construct reporter constructs encoding other reporter genes, for example, a CTL target epitope. Nucleotides 118 and 119 in pG5CAT are changed from AA to CC by site-directed mutagenesis to create an NcoI site at amino acid 1 (aa1) of the CAT gene. Nucleotide 635 is mutagenized from C to T to destroy the NcoI site at aa173 of the CAT gene. A gene encoding a CTL epitope or other reporter protein is then be cloned into the modified pG5 vector between the newly-engineered NcoI site, and the B spEl site starting at the codon for amino acid 71 of CAT. The transcribed mRNA is a fusion product of the reporter gene upstream of the 3′ coding sequence that encodes the last 150 amino acid residues of CAT. However, a translational stop signal at the 3′ terminus of the reporter sequence prevents undesired translation of the CAT fragment.

### EXAMPLE 3

### Selection of Specific Human Intrabodies from a cDNA Library Constructed in Adenovirus, Herpesvirus, or Retrovirus Vectors

3.1 Herpesvirus. A method has been described for the generation of helper virus free stocks of recombinant, infectious Herpes Simplex Virus Amplicons (T. A. Stavropoulos, C. A. Strathdee. 1998 J. Virology 72:7137-7143). According to this method, a cDNA library of human immunoglobulin heavy and/or light chain genes (or fragments thereof) or single-chain fragments are constructed in the plasmid Amplicon vector, and packaged into a library of infectious amplicon particles. An Amplicon library constructed using immunoglobulin heavy chain genes or fragments, and another Amplicon library constructed using immunoglobulin light chain genes or fragments are used to coinfect a non-producing myeloma cell line. Alternatively, the Amplicon library is constructed using polynucleotides encoding single-chain fragments, and only one library and screening/selection step is necessary). The Herpes Amplicons are capable of stable transgene expression in infected cells. The myeloma cells expressing an immunoglobulin gene combination which modifies a phenotype are enriched by screening or selection, using methods described herein, including selection strategies that result in cell death.

Cells screened for or selected in a first cycle retain their immunoglobulin gene combination, and stably express the antibody. This allows for the reiteration of selection cycles until desired immunoglobulin genes are isolated. The amplicon vector recovered from dead selected cells cannot be used to infect fresh target cells, because in the absence of helper virus the amplicons are replication defective and will not be packaged into infectious form. The amplicon vectors contain a plasmid origin of replication and an antibiotic resistance gene. This makes it possible to recover the selected/screened amplicon vector by transforming DNA purified from the selected/screened cells into bacteria. Selection with the appropriate antibiotic allows for the isolation of bacterial cells that are transformed by the amplicon vector. The use of different antibiotic resistance genes on the heavy and light chain Amplicon vectors, for example ampicillin and kanamycin, allows for the separate selection of heavy and light chain genes from the same population of selected cells.

Amplicon plasmid DNA is extracted from the bacteria and packaged into infectious viral particles by cotransfection of the amplicon DNA and packaging defective HSV genomic DNA into packaging cells. Infectious amplicon particles are then harvested and used to infect a fresh population of target cells for another round of selection.

3.2 Adenovirus. Methods have been described for the production of recombinant Adenovirus (S. Miyake, M. Makimura, Y. Kanegae, S. Harada, Y. Sato, K. Takamori, C. Tokuda, I. Saito. 1996 Proc. Natl. Acad. Sci. USA 93: 1320-1324; T. C. He, S. Zhou, L. T. Da Costa, J. Yu, K. W. Kinzler, B. Volgelstein. 1998 Proc. Natl. Acad. Sci. USA 95: 2509-2514) According to either of these methods, a cDNA library is constructed in an Adenovirus vector. Insertion of cDNA into the E3 or E4 region of Adenovirus results in a replication competent recombinant virus. This library is used for similar applications as the vaccinia cDNA libraries constructed by trimolecular recombination. For example a heavy chain cDNA library is inserted into the E3 or E4 region of Adenovirus. This results in a replication competent heavy chain library. A light chain cDNA library is inserted into the E1 gene of Adenovirus, generating a replication defective library. This replication defective light chain library is amplified by infection of cells that provide Adenovirus E1 in trans, such as 293 cells. These two libraries (or alternatively, a single-chain fragment library) are used in similar selection strategies as those described using replication competent vaccinia heavy chain library and Psoralen inactivated vaccinia light chain library.

3.3 Advantages of vaccinia virus. Vaccinia virus possesses several advantages over Herpes or Adenovirus for construction of cDNA Libraries. First, vaccinia virus replicates in the cytoplasm of the host cell, while HSV and Adenovirus replicate in the nucleus. A higher frequency of cDNA recombinant transfer plasmid may be available for recombination in the cytoplasm with vaccinia than is able to translocate into the nucleus for packaging/recombination in HSV or Adenovirus. Second, vaccinia virus, but not Adenovirus or Herpes virus, is able to replicate plasmids in a sequence independent manner (M. Merchlinsky, B. Moss. 1988 Cancer Cells 6: 87-93). Vaccinia replication of cDNA recombinant transfer plasmids may result in a higher frequency of recombinant virus being produced.

3.4 Retrovirus. Construction of cDNA Libraries in replication defective retroviral vectors have been described (T. Kitamura, M. Onishi, S. Kinoshita, A. Shibuya, A. Miyajima, and G. P. Nolan. 1995 PNAS 92:9146-9150; I. Whitehead, H. Kirk, and R. Kay. 1995 Molecular and Cellular Biology 15: 704-710.). Retroviral vectors integrate upon infection of target cells, and have gained widespread use for their ability to efficiently transduce target cells, and for their ability to induce stable transgene expression. A Retroviral cDNA library is constructed using immunoglobulin heavy chain genes, and another Retroviral library is constructed using immunoglobulin light chain genes. These are then used to coinfect anon-producing myeloma cell line. Alternatively, a single-chain fragment library is constructed and used herein. The myeloma cells expressing an immunoglobulin, or fragment thereof, with the desired specificity is enriched for by selection or screening for a modified phenotype. Cells selected or screened for in a first cycle retain their immunoglobulin gene combination, and stably express the desired immunoglobulins. This allows for the reiteration of selection cycles until desired immunoglobulin genes can be isolated.

### EXAMPLE 4

### Trimolecular Recombination

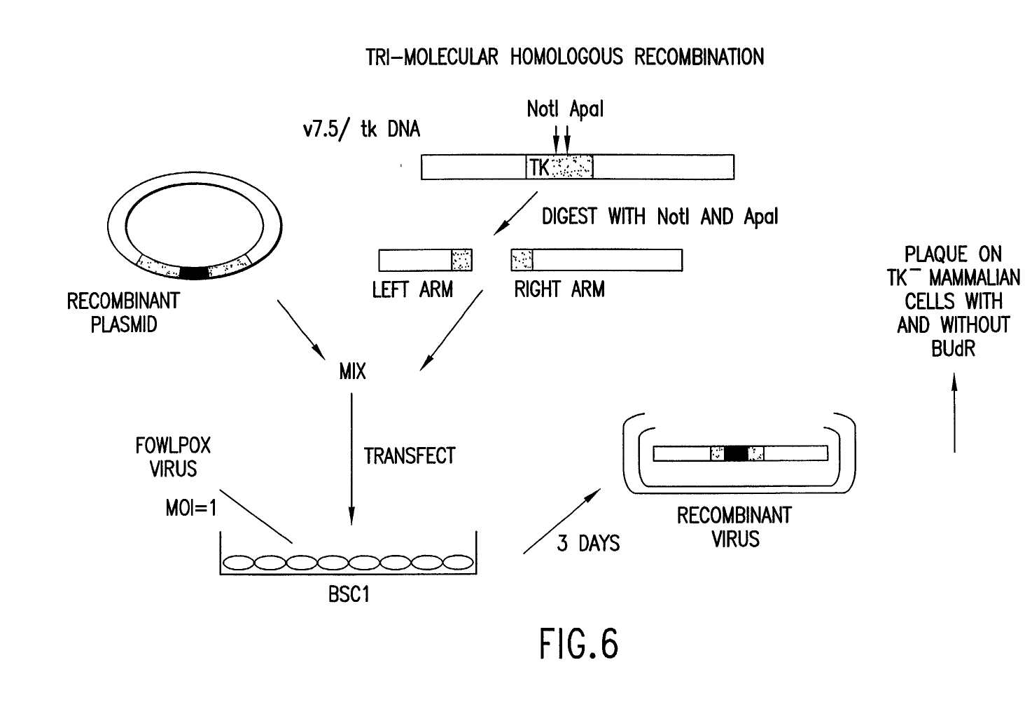

4.1 Production of an Expression Library. This example describes a tri-molecular recombination method employing modified vaccinia virus vectors and related transfer plasmids that generates close to 100% recombinant vaccinia virus and, for the first time, allows efficient construction of a representative DNA library in vaccinia virus. The trimolecular recombination method is illustrated in FIG. 6.

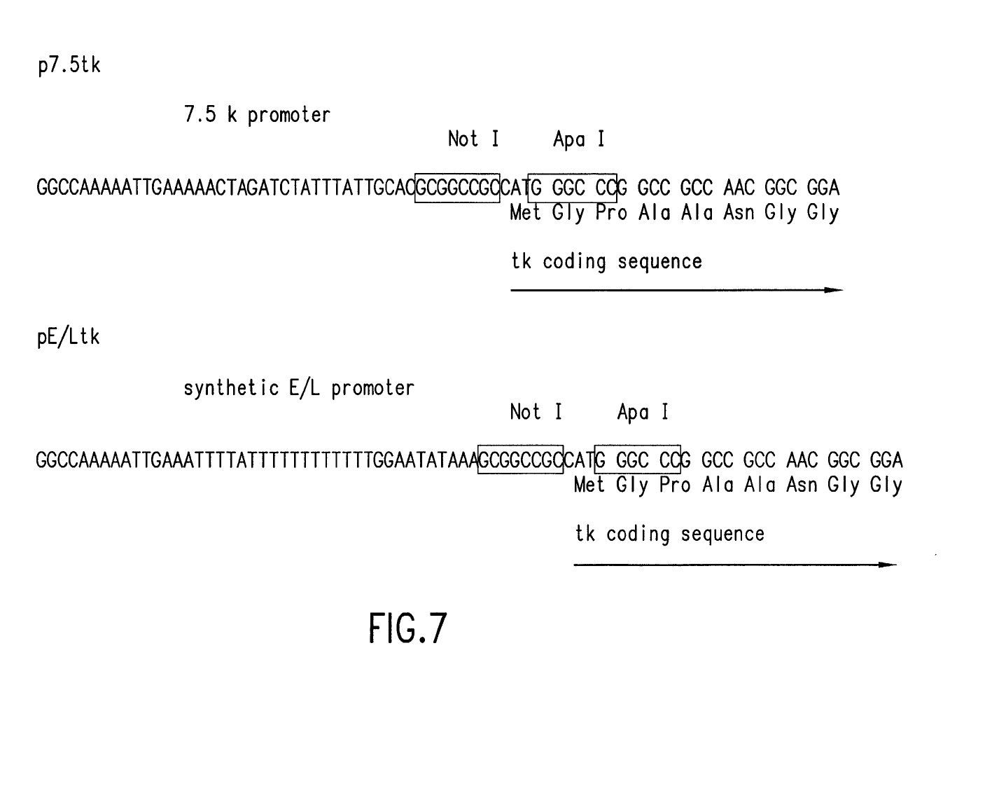

4.2 Construction of the Vectors. The previously described vaccinia virus transfer plasmid pJ/K, a pUC 13 derived plasmid with a vaccinia virus thymidine kinase gene containing an in-frame Not I site (Merchlinsky, M. et al., Virology 190:522-526), was further modified to incorporate a strong vaccinia virus promoter followed by Not I and Apa I restriction sites. Two different vectors, p7.5/tk and pEL/tk, included, respectively, either the 7.5K vaccinia virus promoter or a strong synthetic early/late (E/L) promoter (FIG. 7). The Apa I site was preceded by a strong translational initiation sequence including the ATG codon. This modification was introduced within the vaccinia virus thymidine kinase (tk) gene so that it was flanked by regulatory and coding sequences of the viral tk gene. The modifications within the tk gene of these two new plasmid vectors were transferred by homologous recombination in the flanking tk sequences into the genome of the Vaccinia Virus WR strain derived vNotI⁻ vector to generate new viral vectors v7.5/tk and vEL/tk. Importantly, following Not I and Apa I restriction endonuclease digestion of these viral vectors, two large viral DNA fragments were isolated each including a separate non-homologous segment of the vaccinia tk gene and together comprising all the genes required for assembly of infectious viral particles. Further details regarding the construction and characterization of these vectors and their alternative use for direct ligation of DNA fragments in vaccinia virus are described in Zauderer, WO 00/028016, published May 18, 2000.

4.3 Generation of an Increased Frequency of Vaccinia Virus Recombinants. Standard methods for generation of recombinants in vaccinia virus exploit homologous recombination between a recombinant vaccinia transfer plasmid and the viral genome. Table 5 shows the results of a model experiment in which the frequency of homologous recombination following transfection of a recombinant transfer plasmid into vaccinia virus infected cells was assayed under standard conditions. To facilitate functional assays, a minigene encoding the immunodominant 257-264 peptide epitope of ovalbumin in association with H-2K^{b} was inserted at the Not 1 site in the transfer plasmid tk gene. As a result of homologous recombination, the disrupted tk gene is substituted for the wild type viral tk+ gene in any recombinant virus. This serves as a marker for recombination since tk− human 143B cells infected with tk− virus are, in contrast to cells infected with wild type tk+ virus, resistant to the toxic effect of BrdU. Recombinant virus can be scored by the viral pfu on 143B cells cultured in the presence of 125 mM BrdU.

The frequency of recombinants derived in this fashion is of the order of 0.1% (Table 5).

TABLE 5
Generation of Recombinant Vaccinia Virus
by Standard Homologous Recombination

| | | Titer w/o | Titer w/ | % |
|---|---|---|---|---|
| Virus* | DNA | BrdU | BrdU | Recombinant** |
| vaccinia | — | 4.6 × 10⁷ | 3.0 × 10³ | 0.006 |
| vaccinia | 30 ng pE/Lova | 3.7 × 10⁷ | 3.2 × 10⁴ | 0.086 |
| vaccinia | 300 ng pE/Lova | 2.7 × 10⁷ | 1.5 × 10⁴ | 0.056 |

*vaccinia virus strain vNotI
**% Recombinant = (Titer with BrdU/Titer without BrdU) × 100

This recombination frequency is too low to permit efficient construction of a cDNA library in a vaccinia vector. The following two procedures were used to generate an increased frequency of vaccinia virus recombinants.

(1) One factor limiting the frequency of viral recombinants generated by homologous recombination following transfection of a plasmid transfer vector into vaccinia virus infected cells is that viral infection is highly efficient whereas plasmid DNA transfection is relatively inefficient. As a result many infected cells do not take up recombinant plasmids and are, therefore, capable of producing only wild type virus. In order to reduce this dilution of recombinant efficiency, a mixture of naked viral DNA and recombinant plasmid DNA was transfected into Fowl Pox Virus (FPV) infected mammalian cells. As previously described by others (Scheiflinger, F., et al., 1992, Proc. Natl. Acad. Sci. USA 89:9977-9981), FPV does not replicate in mammalian cells but provides necessary helper functions required for packaging mature vaccinia virus particles in cells transfected with non-infectious naked vaccinia DNA. This modification of the homologous recombination technique alone increased the frequency of viral recombinants approximately 35 fold to 3.5% (Table 6).

TABLE 6
Generation of Recombinant Vaccinia Virus
by Modified Homologous Recombinantion

| | | Titer w/o | Titer w/ | % |
|---|---|---|---|---|
| Virus | DNA | BrdU | BrdU | Recombinant* |
| PFV | None | 0 | 0 | 0 |
| None | vaccinia WR | 0 | 0 | 0 |
| PFV | vaccinia WR | 8.9 × 10⁶ | 2.0 × 10² | 0.002 |
| PFV | vaccinia WR + | 5.3 × 10⁶ | 1.2 × 10⁵ | 2.264 |
| | pE/Lova (1:1) | | | |
| PFV | vaccinia WR + | 8.4 × 10⁵ | 3.0 × 10⁴ | 3.571 |
| | pE/Lova (1:10) | | | |

*% Recombinant = (Titer with BrdU/Titer without BrdU) × 100

Table 6. Confluent monolayers of BSC1 cells (5×10⁵ cells/well) were infected with moi=1.0 of fowlpox virus strain HP1. Two hours later supernatant was removed, cells were washed 2× with Opti-Mem I media, and transfected using lipofectamine with 600 ng vaccinia strain WR genomic DNA either alone, or with 1:1 or 1:10 (vaccinia:plasmid) molar ratios of plasmid pE/Lova. This plasmid contains a fragment of the ovalbumin cDNA, which encodes the SIINFEKL epitope, known to bind with high affinity to the mouse class I MHC molecule K^{b}. Expression of this minigene is controlled by a strong, synthetic Early/Late vaccinia promoter. This insert is flanked by vaccinia tk DNA. Three days later cells were harvested, and virus extracted by three cycles of freeze/thaw in dry ice isopropanol/37° C. water bath. Crude virus stocks were titered by plaque assay on human TK-143B cells with and without BrdU.

(2) A further significant increase in the frequency of viral recombinants was obtained by transfection of FPV infected cells with a mixture of recombinant plasmids and the two large approximately 80 kilobases and 100 kilobases fragments of vaccinia virus v7.5/tk DNA produced by digestion with Not I and Apa I restriction endonucleases. Because the Not I and Apa I sites have been introduced into the tk gene, each of these large vaccinia DNA arms includes a fragment of the tk gene. Since there is no homology between the two tk gene fragments, the only way the two vaccinia arms can be linked is by bridging through the homologous tk sequences that flank the inserts in the recombinant transfer plasmid. The results in Table 7 show that >99% of infectious vaccinia virus produced in triply transfected cells is recombinant for a DNA insert as determined by BrdU resistance of infected tk− cells.

TABLE 7
Generation of 100% Recombinant Vaccinia Virus
Using Tri-Molecular Recombinantion

| | | Titer w/o | Titer w/ | % |
|---|---|---|---|---|
| Virus | DNA | BrdU | BrdU | Recombinant* |
| PFV | Uncut v7.5/tk | 2.5 × 10⁶ | 6.0 × 10³ | 0.24 |
| PFV | NotI/ApaI v7.5/tk arms | 2.0 × 10² | 0 | 0 |
| PFV | NotI/Apal v7.5/tk arms + | 6.8 × 10⁴ | 7.4 × 10⁴ | 100 |
| | pE/Lova (1:1) | | | |

*% Recombinant = (Titer with BrdU/Titer without BrdU) × 100

Table 7. Genomic DNA from vaccinia strain V7.5/tk (1.2 micrograms) was digested with ApaI and NotI restriction endonucleases. The digested DNA was divided in half. One of the pools was mixed with a 1:1 (vaccinia:plasmid) molar ratio of pE/Lova. This plasmid contains a fragment of the ovalbumin cDNA, which encodes the SIINFEKL epitope, known to bind with high affinity to the mouse class I MHC molecule K^{b}. Expression of this minigene is controlled by a strong, synthetic Early/Late vaccinia promoter. This insert is flanked by vaccinia tk DNA. DNA was transfected using lipofectamine into confluent monolayers (5×10⁵ cells/well) of BSC1 cells, which had been infected 2 hours previously with moi=1.0 FPV. One sample was transfected with 600 ng untreated genomic V7.5/tk DNA. Three days later cells were harvested, and the virus was extracted by three cycles of freeze/thaw in dry ice isopropanol/37° C. water bath. Crude viral stocks were plaqued on TK− 143 B cells with and without BrdU selection.

4.4 Construction of a Representative cDNA Library in Vaccinia Virus. A cDNA library is constructed in the vaccinia vector to demonstrate representative expression of known cellular mRNA sequences. Additional modifications have been introduced into the p7.5/tk transfer plasmid and v7.5/tk viral vector to enhance the efficiency of recombinant expression in infected cells. These include introduction of translation initiation sites in three different reading frames and of both translational and transcriptional stop signals as well as additional restriction sites for DNA insertion.

First, the HindIII J fragment (vaccinia tk gene) of p7.5/tk was subcloned from this plasmid into the HindIII site of pBS phagemid (Stratagene) creating pBS.Vtk.

Second, a portion of the original multiple cloning site of pBS.Vtk was removed by digesting the plasmid with SmaI and PstI, treating with Mung Bean Nuclease, and ligating back to itself, generating pBS.Vtk.MCS−. This treatment removed the unique SmaI, BamHI, SalI, and PstI sites from pBS.Vtk.

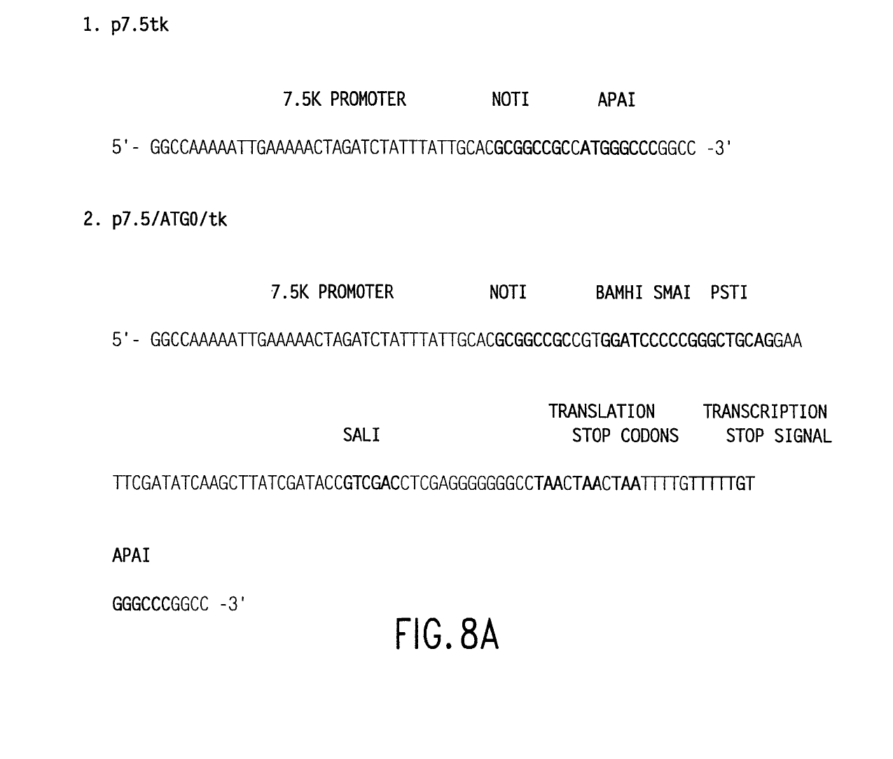

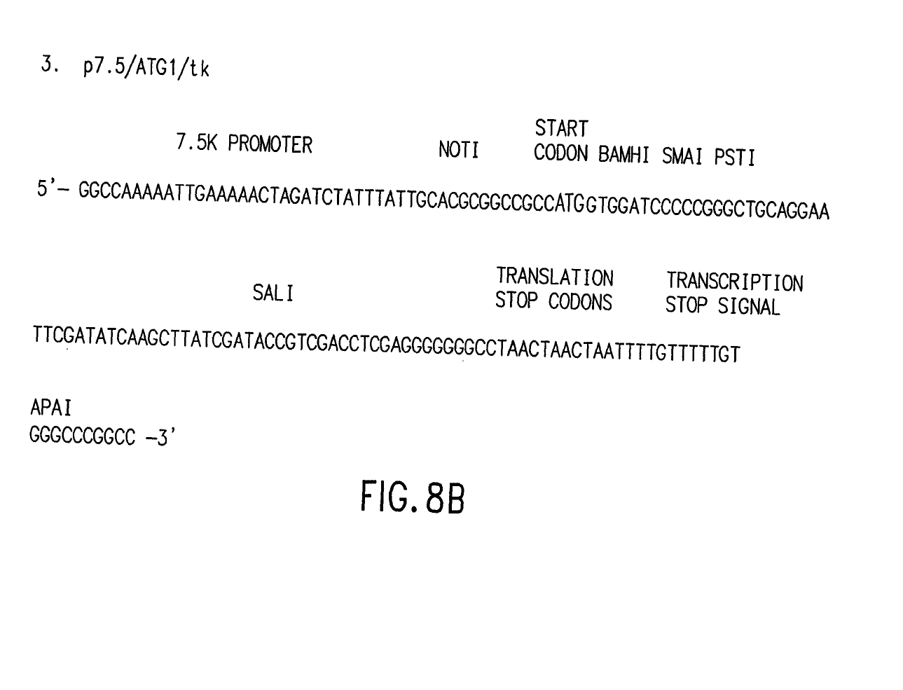

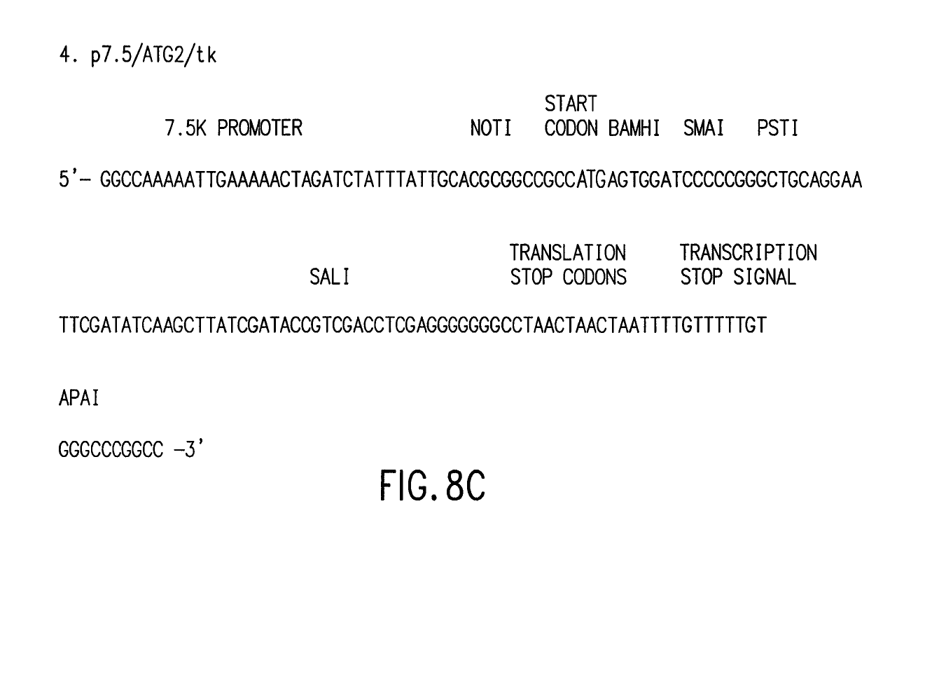

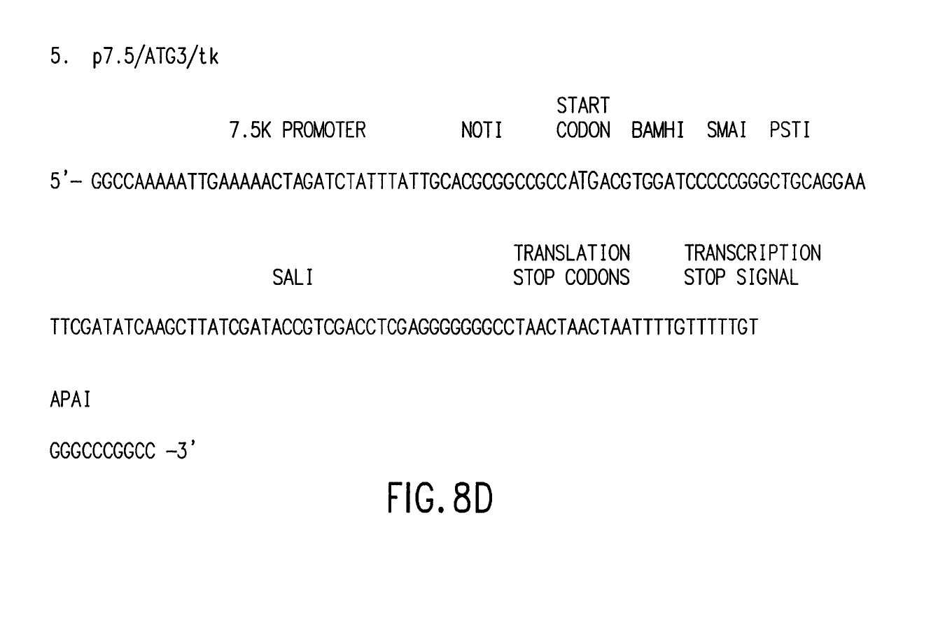

Third, the object at this point was to introduce a new multiple cloning site downstream of the 7.5k promoter in pBS.Vtk.MCS−. The new multiple cloning site was generated by PCR using 4 different upstream primers, and a common downstream primer. Together, these 4 PCR products would contain either no ATG start codon, or an ATG start codon in each of the three possible reading frames. In addition, each PCR product contains at its 3 prime end, translation stop codons in all three reading frames, and a vaccinia virus transcription double stop signal. These 4 PCR products were ligated separately into the NotI/ApaI sites of pBS.Vtk.MCS−, generating the 4 vectors, p7.5/ATG0/tk, p7.5/ATG1/tk, p7.5/ATG3/tk, and p7.5/ATG4/tk whose sequence modifications relative to the p7.5/tk vector are shown in FIG. 8. Each vector includes unique BamHI, SmaI, PstI, and SalI sites for cloning DNA inserts that employ either their own endogenous translation initiation site (in vector p7.5/ATG0/tk) or make use of a vector translation initiation site in any one of the three possible reading frames (p7.5/ATG1/tk, p7.5/ATG3/tk, and p7.5/ATG4/tk).

In a model experiment cDNA was synthesized from poly-A+ mRNA of a murine tumor cell line (BCA39) and ligated into each of the four modified p7.5/tk transfer plasmids. The transfer plasmid is amplified by passage through procaryotic host cells such as E. coli as described herein or as otherwise known in the art. Twenty micrograms of Not I and Apa I digested v/tk vaccinia virus DNA arms and an equimolar mixture of the four recombinant plasmid cDNA libraries was transfected into FPV helper virus infected BSC-1 cells for tri-molecular recombination. The virus harvested had a total titer of 6×10⁶ pfu of which greater than 90% were BrdU resistant.

In order to characterize the size distribution of cDNA inserts in the recombinant vaccinia library, individual isolated plaques were picked using a sterile pasteur pipette and transferred to 1.5 ml tubes containing 100 μl Phosphate Buffered Saline (PBS). Virus was released from the cells by three cycles of freeze/thaw in dry ice/isopropanol and in a 37° C. water bath. Approximately one third of each virus plaque was used to infect one well of a 12 well plate containing tk− human 143B cells in 250 μl final volume. At the end of the two hour infection period each well was overlayed with 1 ml DMEM with 2.5% fetal bovine serum (DMEM-2.5) and with BUdR sufficient to bring the final concentration to 125 μg/ml. Cells were incubated in a CO₂ incubator at 37° C. for three days. On the third day the cells were harvested, pelleted by centrifugation, and resuspended in 500 μl PBS. Virus was released from the cells by three cycles of freeze/thaw as described above. Twenty percent of each virus stock was used to infect a confluent monolayer of BSC-1 cells in a 50 mm tissue culture dish in a final volume of 3 ml DMEM-2.5. At the end of the two hour infection period the cells were overlayed with 3 ml of DMEM-2.5. Cells were incubated in a CO₂ incubator at 37° C. for three days. On the third day the cells were harvested, pelleted by centrifugation, and resuspended in 300 μl PBS. Virus was released from the cells by three cycles of freeze/thaw as described above. One hundred microliters of crude virus stock was transferred to a 1.5 ml tube, an equal volume of melted 2% low melting point agarose was added, and the virus/agarose mixture was transferred into a pulsed field gel sample block. When the agar worms were solidified they were removed from the sample block and cut into three equal sections. All three sections were transferred to the same 1.5 ml tube, and 250 μl of 0.5 M EDTA, 1% Sarkosyl, 0.5 mg/ml Proteinase K was added. The worms were incubated in this solution at 37° C. for 24 hours. The worms were washed several times in 500 μl 0.5× TBE buffer, and one section of each worm was transferred to a well of a 1% low melting point agarose gel. After the worms were added the wells were sealed by adding additional melted 1% low melting point agarose. This gel was then electorphoresed in a Bio-Rad pulsed field gel electrophoresis apparatus at 200volts, 8 second pulse times, in 0.5× TBE for 16 hours. The gel was stained in ethidium bromide, and portions of agarose containing vaccinia genomic DNA were excised from the gel and transferred to a 1.5 ml tube. Vaccinia DNA was purified from the agarose using β-Agarase (Gibco) following the recommendations of the manufacturer. Purified vaccinia DNA was resuspended in 50 μl ddH₂O. One microliter of each DNA stock was used as the template for a Polymerase Chain Reaction (PCR) using vaccinia TK specific primers MM428 and MM430 (which flank the site of insertion) and Klentaq Polymerase (Clontech) following the recommendations of the manufacturer in a 20 μl final volume. Reaction conditions included an initial denaturation step at 95° C. for 5 minutes, followed by 30 cycles of: 94° C. 30 seconds, 55° C. 30 seconds, 68° C. 3 minutes. Two and a half microliters of each PCR reaction was resolved on a 1% agarose gel, and stained with ethidium bromide. Amplified fragments of diverse sizes were observed. When corrected for flanking vector sequences amplified in PCR the inserts range in size between 300 and 2500 bp.

Representative expression of gene products in this library was established by demonstrating that the frequency of specific cDNA recombinants in the vaccinia library was indistinguishable from the frequency with which recombinants of the same cDNA occur in a standard plasmid library. This is illustrated in Table 8 for an IAP sequence that was previously shown to be upregulated in murine tumors.

Twenty separate pools with an average of either 800 or 200 viral pfu from the vaccinia library were amplified by infecting microcultures of 143B tk− cells in the presence of BDUR. DNA was extracted from each infected culture after three days and assayed by PCR with sequence specific primers for the presence of a previously characterized endogenous retrovirus (IAP, intracisternal A particle) sequence. Poisson analysis of the frequency of positive pools indicates a frequency of one IAP recombinant for approximately every 500 viral pfu (Table 8). Similarly, twenty separate pools with an average of either 1,400 or 275 bacterial cfu from the plasmid library were amplified by transformation of DH5 a bacteria. Plasmid DNA from each pool was assayed for the presence of the same IAP sequence. Poisson analysis of the frequency of positive pools indicates a frequency of one IAP recombinant for every 450 plasmids (Table 8).

TABLE 8
Limiting dilution analysis of IAP sequences in a recombinant
Vaccinia library and a conventional plasmid cDNA library

| | #Wells Positive | | | |
|---|---|---|---|---|
| | by PCR | F₀ | μ | Frequency |
| #PFU/well | Vaccinia Library | | | |
| 800 | 18/20 | 0.05 | 2.3 | 1/350 |
| 200 | 6/20 | 0.7 | 0.36 | 1/560 |
| #CFU/well | Plasmid Library | | | |
| 1400 | 20/20 | 0 | — | — |
| 275 | 9/20 | 0.55 | 0.6 | 1/450 |

F₀ = fraction negative wells; μ = DNA precursors/well '2 −lnF₀

Similar analysis was carried out with similar results for representation of an alpha tubulin sequence in the vaccinia library. The comparable frequency of arbitrarily chosen sequences in the two libraries constructed from the same tumor cDNA suggests that although construction of the Vaccinia library is somewhat more complex and is certainly less conventional than construction of a plasmid library, it is equally representative of tumor cDNA sequences.

Discussion

The above-described tri-molecular recombination strategy yields close to 100% viral recombinants. This is a highly significant improvement over current methods for generating viral recombinants by transfection of a plasmid transfer vector into vaccinia virus infected cells. This latter procedure yields viral recombinants at a frequency of the order of only 0.1%. The high yield of viral recombinants in tri-molecular recombination makes it possible, for the first time, to efficiently construct genomic or cDNA libraries in a vaccinia virus derived vector. In the first series of experiments a titer of 6×10⁶ recombinant virus was obtained following transfection with a mix of 20 micrograms of Not I and Apa I digested vaccinia vector arms together with an equimolar concentration of tumor cell cDNA. This technological advance creates the possibility of new and efficient screening and selection strategies for isolation of specific genomic and cDNA clones.

The tri-molecular recombination method as herein disclosed may be used with other viruses such as mammalian viruses including vaccinia and herpes viruses. Typically, two viral arms which have no homology are produced. The only way that the viral arms can be linked is by bridging through homologous sequences that flank the insert in a transfer vector such as a plasmid. When the two viral arms and the transfer vector are present in the same cell the only infectious virus produced is recombinant for a DNA insert in the transfer vector.

Libraries constructed in vaccinia and other mammalian viruses by the tri-molecular recombination method of the present invention may have similar advantages to those described here for vaccinia virus and its use in identifying target antigens in the CTL screening system of the invention. Similar advantages are expected forDNA libraries constructed in vaccinia or other mammalian viruses when carrying out more complex assays in eukaryotic cells. Such assays include but are not limited to screening for DNA encoding receptors and ligands of eukaryotic cells.

### EXAMPLE 5

### Preparation of Transfer Plasmids

The transfer vectors may be prepared for cloning by known means. A preferred method involves cutting 1-5 micrograms of vector with the appropriate restriction endonucleases (for example SmaI and SalI or BamHI and SalI) in the appropriate buffers, at the appropriate temperatures for at least 2 hours. Linear digested vector is isolated by electrophoresis of the digested vector through a 0.8% agarose gel. The linear plasmid is excised from the gel and purified from agarose using methods that are well known.

Ligation. The cDNA and digested transfer vector are ligated together using well known methods. In a preferred method 50-100 ng of transfer vector is ligated with varying concentrations of cDNA using T4 DNA Ligase, using the appropriate buffer, at 14° C. for 18 to 24 hours.

Transformation. Aliquots of the ligation reactions are transformed by electroporation into E. coli bacteria such as DH10 B or DH5 alpha using methods that are well known. The transformation reactions are plated onto LB agar plates containing a selective antibiotic (ampicillin) and grown for 14-18hours at 37° C. All of the transformed bacteria are pooled together, and plasmid DNA is isolated using well known methods.

Preparation of buffers mentioned in the above description of preferred methods according to the present invention will be evident to those of skill.

### EXAMPLE 6

### Introduction of Vaccinia Virus DNA Fragments and Transfer Plasmids into Tissue Culture Cells for Trimolecular Recombination

Libraries of cDNA encoding intracellular immunoglobulin subunit polypeptides, or fragments thereof, are constructed using the various transfer plasmids described in such as those described in Example 1, or by other art-known techniques. Trimolecularrecombination is employed to transferthis cDNA library into vaccinia virus. Confluent monolayers ofBSC1 cells are infected with fowlpox virus HP1 at a moi of 1-1.5. Infection is done in serum free media supplemented with 0.1% Bovine Serum Albumin. The BSC1 cells may be in 12 well or 6 well plates, 60 mm or 100 mm tissue culture plates, or 25 cm², 75 cm², or 150 cm² flasks. Plasmids carrying the coding regions for intracellular immunoglobulin subunit polypeptides are digested with restriction endonucleases ApaI and NotI. Following these digestions the enzymes are heat inactivated, and the digested vaccinia arms are purified using a centricon 100 column. Transfection complexes are then formed between the digested vaccinia DNA and the transfer plasmid cDNA library. A preferred method uses Lipofectamine or Lipofectamine Plus (Life Technologies, Inc.) to form these transfection complexes. Transfections in 12 well plates usually require 0.5 micrograms of digested vaccinia DNA and 10 ng to 200 ng of plasmid DNA from the library. Transfection into cells in larger culture vessels requires a proportional increase in the amounts of vaccinia DNA and transfer plasmid. Following a two hour infection at 37° C. the fowlpox is removed, and the vaccinia DNA, transfer plasmid transfection complexes are added. The cells are incubated with the transfection complexes for 3 to 5 hours, after which the transfection complexes are removed and replaced with 1 ml DMEM supplemented with 2.5% Fetal Bovine Serum. Cells are incubated in a CO₂ incubated at 37° C. for 3 days. After 3 days the cells are harvested, and virus is released by three cycles of freeze/thaw in dry ice/isopropanol/37° C. water bath.

### EXAMPLE 7

### Transfection of Mammalian Cells

This example describes alternative methods to transfect cells with vaccinia DNA and transfer plasmid. Trimolecular recombination can be performed by transfection of digested vaccinia DNA and transfer plasmid into host cells using for example, calcium-phosphate precipitation [Graham, F. L., et al., Virology 52: 456-467 (1973); Chen, C., et al., Mol. Cell. Biol. 7:2745-2752 (1987)], DEAE-Dextran [Sussman, D. J., et al., Mol. Cell. Biol. 4:1641-1643 (1984)], or electroporation [Wong, T. K., Biochem. Biophys. Res. Commun. 107:584-587 (1982); Neumann, E., et al., EMBO J. 1: 841-845 (1982)].

### EXAMPLE 8

### Construction of MVA Trimolecular Recombination Vectors

In order to construct a Modified Vaccinia Ankara (MVA) vector suitable for trimolecular recombination, two unique restriction endonuclease sites are inserted into the MVA tk gene. The complete MVA genome sequence is known (GenBank U94848). A search of this sequence revealed that restriction endonucleases AscI, RsrII, SfiI, and XmaI do not cut the MVA genome. Restriction endonucleases AscI and XmaI have been selected due to the commercial availability of the enzymes, and the size of the recognition sequences, 8 bp and 6 bp for AscI and XmaI respectively. In order to introduce these sites into the MVA tk gene a construct is made that contains a reporter gene (E. coli gusA) flanked by XmaI and AscI sites. The Gus gene is available in pCRII.Gus (M. Merchlinsky, D. Eckert, E. Smith, M. Zauderer. 1997 Virology 238:444-451). This reporter gene construct is cloned into a transfer plasmid containing vaccinia tk DNA flanks and the early/late 7.5k promoter to control expression of the reporter gene. The Gus gene is PCR amplified from this construct using Gus specific primers. Gus sense 5′ ATGTTACGTCCTGTAGAAACC 3′ (SEQ ID NO:126), and Gus Antisense 5′TCATTGTTTGCCTCCCTGCTG 3′(SEQ ID NO:127). The Gus PCR product is then PCR amplified with Gus specific primers that have been modified to include NotI and XmaI sites on the sense primer, and AscI and Apal sites on the antisense primer. The sequence of these primers is:

| | |
|---|---|
| NX-Gus Sense | |
| 5′ AAAGCGGCCGCCCCGGGATGTTACGTC | (SEQ ID NO:128) |
| C 3′; and | |
| AA-Gus antisense | |
| 5′ AAAGGGCCCGGCGCGCCTCATTGTTTG | (SEQ ID NO:129) |
| CC 3′. | |

This PCR product is digested with NotI and ApaI and cloned into the NotI and ApaI sites of p7.5/tk (M. Merchlinsky, D. Eckert, E. Smith, M. Zauderer. 1997 Virology 238: 444-451). The 7.5k-XmaI-gusA-AscI construct is introduced into MVA by conventional homologous recombination in permissive QT35 or BHK cells. Recombinant plaques are selected by staining with the Gus substrate X-Glu (5-bromo-3 indoyl-β-D-glucuronic acid; Clontech) (M. W. Carroll, B. Moss. 1995 Biotechniques 19:352-355). MVA-Gus clones, which also contain the unique XmaI and AscI sites, are plaque purified to homogeneity. Large scale cultures of MVA-Gus are amplified on BHK cells, and naked DNA is isolated from purified virus. After digestion with XmaI and AscI the MVA-Gus DNA is used for trimolecular recombination so that cDNA expression libraries are constructed in MVA.

MVA is unable to complete its life cycle in most mammalian cells. This attenuation can result in a prolonged period of high levels of expression of recombinant cDNAs, but viable MVA cannot be recovered from infected cells. The inability to recover viable MVA from selected cells prevents the repeated cycles of selection required to isolate functional cDNA recombinants of interest. Infection of MVA infected cells with a helper virus that complements the host range defects of MVA overcomes this problem. This helper virus provides the gene product(s) which MVA lacks that are essential for completion of its life cycle. It is unlikely that another host range restricted helper virus, such as fowlpox, will complement the MVA defect(s), as these viruses are also restricted in mammalian cells. Wild type strains of vaccinia virus are able to complement MVA. In this case however, production of replication competent vaccinia virus complicates additional cycles of selection and isolation of recombinant MVA clones. A conditionally defective vaccinia virus is used to provide the helper function needed to recover viable MVA from mammalian cells under nonpermissive conditions, without the generation of replication competent virus.

The vaccinia D4R open reading frame (orf) encodes a uracil DNA glycosylase enzyme. This enzyme is essential for vaccinia virus replication, is expressed early after infection (before DNA replication), and disruption of this gene is lethal to vaccinia. It has been demonstrated that a stably transfected mammalian cell line expressing the vaccinia D4R gene was able to complement a D4R deficient vaccinia virus (G. W. Holzer, F. G. Falkner. 1997 J. Virology 71: 4997-5002). A D4R deficient vaccinia virus is an excellent candidate as a helper virus to complement MVA in mammalian cells.

In order to construct a D4R complementing cell line the D4R orf is cloned from vaccinia strain v7.5/tk by PCR amplification using primers D4R-Sense 5′ AAAGGATCCA TAATGAATTC AGTGACTGTA TCACACG 3′ (SEQ ID NO:130), and D4R Antisense 5′ CTTGCGGCCG CTTAATAAAT AAACCCTTGA GCCC 3′(SEQ ID NO:131). The sense primer has been modified to include a BamHI site, and the anti-sense primer has been modified to include a NotI site. Following PCR amplification and digestion with BamHI and NotI the D4R orf is cloned into the BamHI and NotI sites of pIRESHyg (Clontech). This mammalian expression vector contains the strong CMV Immediate Early promoter/Enhancer and the ECMV internal ribosome entry site (IRES). The D4RIRESHyg construct is transfected into BSC1 cells and transfected clones are selected with hygromycin. The IRES allows for efficient translation of a polycistronic mRNA that contains the D4Rorf at the 5′ end, and the Hygromycin phosphotransferase gene at the 3′ end. This results in a high frequency of Hygromycin resistant clones being functional (the clones express D4R). BSC1 cells that express D4R (BSC1.D4R) complement D4R deficient vaccinia, allowing for generation and propagation of this defective strain.

To construct D4R deficient vaccinia, the D4R orf (position 100732 to 101388 in vaccinia genome) and 983 bp (5′ end) and 610 bp (3′end) of flanking sequence is PCR amplified from the vaccinia genome. Primers D4R Flank sense 5′ ATTGAGCTCT TAATACTTTT GTCGGGTAAC AGAG 3′ (SEQ ID NO:132), and D4R Flank antisense 5′ TTACTCGAGA GTGTCGCAAT TTGGATTTT 3′ (SEQ ID NO:133) contain a SacI (Sense) and XhoI (Antisense) site for cloning and amplify position 99749 to 101998 of the vaccinia genome. This PCR product is cloned into the SacI and XhoI sites of pBluescript II KS (Stratagene), generating pBS.D4R.Flank. The D4R gene contains a unique EcoRI site beginning at nucleotide position 3 of the 657bp orf, and a unique PstI site beginning at nucleotide position 433 of the orf. Insertion of a Gus expression cassette into the EcoRI and PstI sites of D4R removes most of the D4R coding sequence. A 7.5k promoter-Gus expression vector has been constructed (M. Merchlinsky, D. Eckert, E. Smith, M. Zauderer. 1997 Virology 238: 444-451). The 7.5-Gus expression cassette is isolated from this vector by PCR using primers 7.5 Gus Sense 5′ AAAGAATTCC TTTATTGTCA TCGGCCAAA 3′ (SEQ ID NO:134) and 7.5Gus antisense 5′ AATCTGCAGT CATTGTTTGC CTCCCTGCTG 3′ (SEQ ID NO:135). The 7.5Gus sense primer contains an EcoRI site and the 7.5Gus antisense primer contains a PstI site. Following PCR amplification the 7.5Gus molecule is digested with EcoRI and PstI and is inserted into the EcoRI and PstI sites in pBS.D4R.Flank, which generates pBS.D4R-/7.5Gus⁺. D4R⁻/Gus⁺ vaccinia is generated by conventional homologous recombination by transfecting the pBS.D4R⁻/7.5Gus⁺ construct into v7.5/tk infected BSC1.D4R cells. D4R⁻/Gus⁺ virus is isolated by plaque purification on BSC1.D4R cells and staining with X-Glu. The D4R− virus is used to complement and rescue the MVA genome in mammalian cells. In a related embodiment, the MVA genome is rescued in mammalian cells with other defective poxviruses, and also by a psoralen/UV-inactivated wild-type poxviruses. Psoralen/UV inactivation is discussed herein.

### EXAMPLE 9

### Construction and Use of D4R Trimolecular Recombination Vectors

Poxvirus infection can have a dramatic inhibitory effect on host cell protein and RNA synthesis. These effects on host gene expression could, under some conditions, interfere with the selection of specific poxvirus recombinants that have a defined physiological effect on the host cell. Some strains of vaccinia virus that are deficient in an essential early gene have been shown to have greatly reduced inhibitory effects on host cell protein synthesis. Therefore, production of recombinant cDNA libraries in a poxvirus vector that is deficient in an early gene function may be advantageous for selection of certain recombinants that depend on continued active expression of some host genes. Disruption of essential viral genes prevents viral replication. Replication defective strains of vaccinia are rescued by providing the missing function through transcomplementation, such as by an host cell-encoded or helper virus-encoded gene under the control of an inducible promoter.

Infection of a cell population with a poxvirus library constructed in a replication deficient strain should greatly attenuate the effects of infection on host cell signal transduction mechanisms, differentiation pathways, and transcriptional regulation. An additional and important benefit of this strategy is that expression of the essential gene under the control of a inducible promoter can itself be the means of selecting recombinant virus that directly or indirectly lead to activation of that transcriptional regulatory region. Examples include the promoter of a gene activated as a result of crosslinking surface immunoglobulin receptors on early B cell precursors or the promoter of a gene that encodes a marker induced following stem cell differentiation. Additional examples of inducible promoters include cell type-restricted promoters, tissue-restricted promoters, temporally-regulated promoters, spatially-regulated promoters, proliferation-induced promoters, cell-cycle specific promoters, etc., such as those described herein or well-known in the art. If such a promoter drives expression of an essential viral gene, then only those viral recombinants that directly or indirectly activate expression of that transcriptional regulator will replicate and be packaged as infectious particles. This method has the potential to give rise to much lower background then selection methods based on expression of dipA or a CTL target epitope because uninduced cells will contain no replication competent vaccinia virus that might be released through non-specific bystander effects. The selected recombinants can be further expanded in a complementing cell line or in the presence of a complementing helper virus or transfected plasmid.

A number of essential early vaccinia genes have been described. Preferably, a vaccinia strain deficient for the D4R gene could be employed. The vaccinia D4R open reading frame (orf) encodes a uracil DNA glycosylase enzyme. This enzyme is reqired for viral DNA replication and disruption of this gene is lethal to vaccinia (A. K. Millns, M. S. Carpenter, and A. M. Delange. 1994 Virology 198:504-513). It has been demonstrated that a stably transfected mammalian cell line expressing the vaccinia D4R gene is able to complement a D4R deficient vaccinia virus (G. W. Holzer, F. G. Falkner. 1997 J. Virology 71: 4997-5002). In the absence of D4R complementation, infection with the D4R deficient vaccinia results in greatly reduced inhibition of host cell protein synthesis (Holzer and Falkner). It has also been shown that a foreign gene inserted into the tk gene of D4R deficient vaccinia continues to be expressed at high levels, even in the absence of D4R complementation (M. Himly, M. Pfleiderer, G. Holzer, U. Fischer, E. Hannak, F. G. Falkner, and F. Dorner. 1998 Protein Expression and Purification 14: 317-326). The replication deficient D4R strain is, therefore, well-suited for selection of viral recombinants that depend on continued active expression of some host genes for their physiological effect.

To implement this strategy for selection of specific recombinants from representative cDNA libraries constructed in a D4R deficient vaccinia strain the following cell lines and vectors are required:

1. D4R expressing complementing cell line for expansion of D4R deficient viral stocks.

2. The D4R defective viral strain suitable for trimolecular recombination.

3. Plasmid or viral constructs that express D4R under the control of different inducible promoters. Stable transfectants of these constructs in relevant cell line are used to rescue specific recombinants. Alternatively, a helper virus expressing the relevant construct can be employed for induction in either cell lines or primary cultures.

9.1. Construction of a D4R Complementing Cell Line. A D4R complementing cell line is constructed as follows. First, the D4R orf (position 100732 to 101388 in vaccinia genome) is cloned from vaccinia strain v7.5/tk by PCR amplification using the following primers:

D4R-sense,

| | |
|---|---|
| designated herein as, | SEQ ID NO:136 |

5′ AAAGAATTCA TAATGAATTC AGTGACTGTA TCACACG 3′;
and D4R-antisense:

| | |
|---|---|
| designated herein as, | SEQ ID NO:137 |

5′ CTTGGATCCT TAATAAATAA ACCCTTGAGC CC 3′.

The sense primer is modified to include an EcoRI site, and the anti-sense primer is modified to include a BamHI site (both underlined). Following standard PCR amplification and digestion with EcoRI and BamHI, the resulting D4R orf is cloned into the EcoRI and BamHI sites of pIRESneo (available from Clontech, Palo Alto, Calif.). This mammalian expression vector contains the strong CMV immediate early promoter/enhancer and the ECMV internal ribosome entry site (IRES). The D4R/IRESneo construct is transfected into BSC1 cells and transfected clones are selected with G418. The IRES allows for efficient translation of a polycistronic mRNA that contains the D4Rorf at the 5′ end, and the neomycin phosphotransferase gene at the 3′ end. This results in a high frequency of G418 resistant clones being functional (the clones express D4R). Transfected clones are tested by northern blot analysis using the D4R gene as probe in order to identify clones that express high levels of D4R mRNA. BSC1 cells that express D4R (BSC1.D4R) are able to complement D4R deficient vaccinia, allowing for generation and propagation of D4R defective viruses.

9.2 Construction of a D4R Deficient vaccinia vector. A D4R-deficient vaccinia virus, suitable for trimolecular recombination as described in Example 4, herein, is constructed by disruption of the D4R orf (position 100732 to 101388 in vaccinia genome) through the insertion of an E. coli GusA expression cassette into a 300-bp deletion, by the following method.

In order to insert the GusA gene, regions flanking the insertion site are amplified from vaccinia virus as follows. The left flanking region is amplified with the following primers:

D4R left flank sense:

| | |
|---|---|
| designated herein as, | SEQ ID NO:138 |

5′ AATAAGCTTT GACTCCAGAT ACATATGGA 3′; and
D4R left flank antisense:

| | |
|---|---|
| designated herein as, | SEQ ID NO:139 |

5′ AATCTGCAGC ACCAGTTCCA TCTTT 3′.

These primers amplify a region extending from position 100167 to position 100960 of the vaccinia genome, and have been modified to include a HindIII (Sense) and PstI (Antisense) site for cloning (both underlined). The resulting PCR product is digested with HindIII and PstI, and cloned into the HindIII and PstI sites of pBS (available from Stratagene), generating pBS.D4R.LF. The right flanking region is amplified with the following primers:

D4R right flank sense:

| | |
|---|---|
| designated herein as, | SEQ ID NO:140 |

5′ AATGGATCCT CATCCAGCGG CTA 3′; and
D4R right flank antisense:

| | |
|---|---|
| designated herein as, | SEQ ID NO:141 |

5′ AATGAGCTCT AGTACCTACA ACCCGAA3′.

These primers amplify a region extending from position 101271 to position 101975 of the vaccinia genome, and have been modified to include a BamHI (Sense) and SacI (Antisense) site for cloning (both underlined). The resulting PCR product is digested with BamHI and SacI, and cloned into the BamHI and SacI sites of pBS.D4R.LF, creating pBS.D4R.LF/RF.

An expression cassette comprising the GusA coding region operably associated with a poxvirus synthetic early/late (E/L) promoter, is inserted into pBS.D4R.LF/RF by the following method. The E/L promoter-Gus cassette is derived from the pEL/tk-Gus construct described in Merchlinsky, M., et al., Virology 238: 444-451 (1997). The NotI site immediately upstream of the Gus ATG start codon is removed by digestion of pEL/tk-Gus with NotI, followed by a fill in reaction with Klenow fragment and religation to itself, creating pEL/tk-Gus(NotI−). The E/L-Gus expression cassette is isolated from pEL/tk-Gus(NotI−) by standard PCR using the following primers:

EL-Gus sense:

| | |
|---|---|
| designated herein as, | SEQ ID NO:142 |

5′ AAAGTCGACG GCCAAAAATT GAAATTTT 3′; and
EL-Gus antisense:

| | |
|---|---|
| designated herein as, | SEQ ID NO:143 |

5′ AATGGATCCT CATTGTTTGC CTCCC 3′.

The EL-Gus sense primer contains a SalI site and the EL-Gus antisense primer contains a BamHI site (both underlined). Following PCR amplification the EL-Gus cassette is digested with SalI and BamHI and inserted into the SalI and BamHI sites in pBS.D4R.LF/RF generating pBS.D4R⁻/ELGus. This transfer plasmid contains an EL-Gus expression cassette flanked on both sides by D4R sequence. There is also a 300 bp deletion engineered into the D4R orf.

D4R⁻/Gus⁺ vaccinia viruses suitable for trimolecular recombination are generated by conventional homologous recombination following transfection of the pBS.D4R⁻/ELGus construct into v7.5/tk-infected BSC1.D4R cells. D4R⁻/Gus⁺ virus are isolated by plaque purification on BSC1.D4R cells and staining with X-Glu (M. W. Carroll, B. Moss. 1995. Biotechniques 19: 352-355). This new strain is designated v7.5/tk/Gus/D4R.

DNA purified from v7.5/tk/Gus/D4R is used to construct representative vaccinia cDNA libraries by the trimolecular recombination method using the BSC1.D4R complementing cell line.

9.3 Preparation of host cells expressing D4R under the control of inducible promoters. Host cells which express the D4R gene upon induction of an inducible promoter are prepared as follows. Plasmid constructs are generated that express the vaccinia D4R gene under the control of an inducible promoter.

Examples of inducible promoters include, but are not limited to the promoter for a marker of differentiation, such as type X collagen. The vaccinia D4R orf is amplified by PCR using primers D4R sense and D4R antisense described above.

These PCR primers are modified as needed to include desirable restriction endonuclease sites. The D4R orf is then cloned in a suitable eukaryotic expression vector (which allows for the selection of stably transformed cells) in operable association of any desired promoter employing methods known to those skilled in the art.

The D4R gene, in operable association with the inducible promoter such as the type X collagen promoter is stably transfected into a suitable cell line, for example, C3H110T1/2 progenitor cells. The resulting host cells are used in the selection, screening, or production of intracellular immunoglobulin molecules, or fragments thereof using libraries prepared in v7.5/tk/Gus/D4R. Differentiation results in the induction of expression of the D4R gene product. Expression of D4R complements the defect in the v7.5tk/Gus/D4R genomes in which the libraries are produced, allowing the production of infectious virus particles.

### EXAMPLE 10

### Intrabodies that Modify Differentiation

Intrabodies identified by methods described herein are useful to identify intracellular regulatory factors. For example, intrabodies are used to identify negative regulators that inhibit differentiation of musculoskeletal stem cells into type X collagen producing chondrocytes. Stem cells are modified to express a genetic construct in which the promoter for type X collagen regulates expression of a gene that directly or indirectly results in cell suicide or binding to a specific substrate. Viral recombinants expressing an intrabody that promotes expression of type X collagen are then recovered from those host cells in which cell death or binding to a specific substrate has been induced. For example, intrabodies are selected that induce stem cell differentiation by blocking a previously unidentified inhibitory factor. The intrabody itself may then be employed to isolate and characterize that inhibitory factor.

A further modification of the method allows intrabodies expressing an appropriate localization signal to be targeted to specific cellular compartments other than the cytoplasm such as the nucleus, plasma membrane, endoplasmic reticulum, mitochondria, lysosomes, or peroxisomes in order to promote interaction with a protein with a selectable phenotype that is expressed in that particular organelle. In combination with Examples 2 and 11, describing the two-hybrid selection strategy, this method enables selection in mammalian cells of intrabodies of predefined antigenic specificity. In this strategy, a known antigen is expressed as a fusion protein with a DNA binding domain, and a library of intrabodies is expressed as fusion proteins with a transcriptional activator, e.g., VP16 as described in Example 2(a). Any intrabody with affinity for the known antigen will associate with the DNA binding domain and together they activate transcription of a reporter gene. For example, the known antigen coding sequence may be fused to the 3′ end of the coding sequence for the DNA binding domain of the yeast Gal4 protein in a mammalian expression vector, and the antibody library coding sequences may be fused to the 5′ or 3′ end of the activation domain of the herpes virus 1 VP16 transcription factor in a vaccinia expression vector. A third construct directs the expression of a reporter gene under the control of a GAL4-responsive element and the minimal promoter of the adenovirus E1b. The cognate interaction between antigen and antibody forms a functional transactivator, which induces expression of the reporter gene. The reporter gene may, for example, encode a CTL target antigen, and the cells are then selected by contacting them with CTLs specific for that target antigen, causing them the become nonadherent, e.g. die, lyse. Polynucleotides encoding the antibody are then recovered from the nonadherent cells.

### EXAMPLE 11

### Direct Selection for Binding Partners Using Two Hybrid System and Suicide Gene Construct or Reporter Gene Construct

The two hybrid system is based on the fact that many eukaryoticotic transcriptional activators are comprised of two physically and functionally separable domains, a DNA-binding domain (DNA-BP) and an activation domain (AD). The two domains are normally part of the same protein. However, the two domains can be separated and expressed as distinct proteins. Two additional proteins (X and Y) are expressed as fusions to the DNA-BP and AD peptides. If X and Y interact, the AD is co-localized to the DNA-BP bound to the promoter, resulting in the transcription of the suicide gene.

The following is an example of the two hybrid transcriptional activation direct selection system. This system is composed of two fusion polynucleotides, one of which may be expressed by a tissue- or cell- or differentiation-specific promoter or a constitutive promoter and the second is found in a poxvirus vector:

1) a fusion of known protein X with the GAL4 DNA-BP;

2) a fusion of a test protein Y with the VP16 activation domain;

where protein X and Y interact (for example, the SV40 large T antigen which associates with the p53 protein). A third construct provides the GAL4 DNA binding site, the minimal promoter of the adenovirus Elb, and the suicide gene.

ES, or any readily transected cells such as Cos 7 or 293 cells, are “seeded” with the first and third constructs either before or after infection with a library cloned in a poxvirus or other vector. The constructs preferably also contain a selectable marker such as PGK neo. The poxvirus vector contains insert polynucleotides fused to the VP16 activation domain preceded by a strong constitutive poxvirus promoter. The inserts may be in each reading frame. The ES cells are cultured and nonviable cells are removed from viable/adherent cells.

Examples of protein binding partners that would be identified using this method are as follows:

1) the GAL4 DNA binding domain fused to the Fos leucine zipper domain (DFosLZ), and

2) the VP16 activation domain fused to the Jun leucine zipper (AJunLZ); or

1) the GAL4 DNA binding domain fused to the Jun leucine zipper domain (DJunLZ), and

2) the VP16 activation domain fused to the Fos leucine zipper (AFosLZ).

The construction of these fusions have been previously described in Dang et al., (1991) Molecular and Cellular Biology 11:954-962, and components to create the vectors of this system (except leucine zipper components) may be obtained from Clontech-Mammalian Matchmaker™ two hybrid assay kit.

An example of a gene system whose expression is dependent on the presence of two interacting fusion proteins is the G5E1b promoter, which contains 5 copies of the 17 mer GAL4 DNA binding site 5′ of the minimal promoter of the Adenovirus Elb, driving the expression of a CAT reporter gene.

This system can be modified as described in Example 2 to select for intrabodies of a defined specificity. Other reporter genes may also be used, such as luciferase, B-galactosidase, green fluorescent protein, and others well-known in the art and disclosed herein. For the selection method by lysis/nonadherence, the CAT gene is replaced by a suicide gene. Additionally, a polynucleotide encoding a CTL antigen may be used in place of the CAT gene, and CTL-mediated lysis/nonadherence may be used to select/screen for intrabodies which bind the known antigen.

### EXAMPLE 12

### Host Cells

Cells and cells lines for use as host or recipient or library cells according to the present invention include those disclosed in scientific literature such as American Type Culture Collection publications including American Type Culture Collection Catalogue of Cell Lines and Hybridomas, 7th Ed., ATCC, Rockville, Md. (1992) and the ATCC internet address <<http://phage.atcc.org/searchengine/all.html>>, which list deposited cell lines as well as culture conditions and additional references.

For example, host cells according to the present invention include the monkey kidney cell line, designated “COS,” including COS cell clone M6. COS cells are those that have been transformed by SV40 DNA containing a functional early gene region but a defective origin of viral DNA replication. Also preferred are murine “WOP” cells, which are NIH 3T3 cells transfected with polyoma origin deletion DNA.

Other examples of host cells for use in the disclosed methods are monkey kidney CVI line transformed by SV40 (COS-7, ATCC CRL 165 1); human embryonic kidney line (293, Graham et al. J. Gen Virol. 36:59 (1977)); baby hamster kidney cells (BHK, ATCC CCL 10); chinese hamster ovary-cells-DHFR (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CVI ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TRI cells (Mather et al., Annals N. Y. Acad. Sci 383:44-68 (1982)); human B cells (Daudi, ATCC CCL 213); human T cells (MOLT-4, ATCC CRL 1582); and human macrophage cells (U-937, ATCC CRL 1593).

Preferred cell types for use in the invention will vary with the desired cellular phenotype to be modified. Suitable cells include, but are not limited to, mammalian cells, including animal (rodents, including mice, rats, hamsters and gerbils), primates, and human cells, particularly including tumor cells of all types, including breast, skin, lung, cervix, colorectal, leukemia, brain, etc.

The murine stem cell line RAW (Hsu, H. et al., Proc Natl Acad Sci USA 96(7):3540-45 (1999); Owens, J. M. et al., J Cell Physiol 179:170 (1999)) and pluripotent stem cell line C3H10T1/2 (Denker, A. et al., Differentiation 64,67-76 (1999)) are especially preferred for studies of osteoclast and chondrocyte or osteoblast differentiation.

However, the choice of cells or cell lines is not limited to those described herein, and may be any cell or cell line. As indicated below, the choice depends on the system under study, or the particular polynucleotide which is desired to be isolated. For example, to isolate an epitope recognized by a human CD8⁺ CTL, it is preferable to use a host cell which expresses human class I MHC molecules, and to isolate an epitope recognized by a human CD4⁺ CTL, it is preferable to use a host cell which expresses human class II MHC molecules, to allow the CTL to recognize the encodedepitope in association with the appropriate MHC molecules. As another example, to isolate a polynucleotide which is growth suppressive or toxic in breast cancer, it is preferable to use as host cells breast cancer cell lines such as 21NT, 21PT, 21MT-1, AND 21MT-2. Band et al., Cancer Res. 50:7351-7 (1990). Once a growth suppressive polynucleotide is isolated, it may be tested in non transformed controls, such as normal breast epithelial cell line H16N2, to determine whether its growth suppressive activity is specific for tumor cells.

Many cell types can be used in the selection method of the invention. Cells include dividing cells, non dividing cells, terminally differentiated cells, pluripotent stem cells, committed progenitor cells and uncommitted stem cells.

Cells and cell types also include muscle cells such as cardiac muscle cells, skeletal muscle cells and smooth muscle cells; epithelial cells such as squamous epithelial cells, including endothelial cells, cuboid epithelial cells and columnar epithelial cells; nervous tissue cells such as neurons and neuroglia.

Cells that can be used in the selection method of the present invention also include nervous system cells such as neurons, including cortical neurons, inter neurons, central effector neurons, peripheral effector neurons and bipolar neurons; and neuroglia, including Schwann cells, oligodendrocytes, astrocytes, microglia and ependyma.

Additionally, endocrine and endocrine-associated cells may also be used such cells as pituitary gland cells including epithelial cells, pituicytes, neuroglia, agranular chromophobes, granular chromophils (acidophils and basophils); adrenal gland cells including epinephrine-secreting cells, non-epinephrine-secreting cells, medullary cells, cortical cells (cells of the glomerulosa, fasciculata and reticularis); thyroid gland cells including epithelial cells (principal and parafollicular); parathyroid gland cells including epithelial cells (chief cells and oxyphils); pancreas cells including cells of the islets of Langerhans (alpha, beta and delta cells); pineal gland cells including parenchymal cells and neuroglial cells; thymus cells including parafollulicular cells; cells of the testes including seminiferous tubule cells, interstitial cells (“Leydig cells”), spermatogonia, spermatocytes (primary and secondary), spermatids, spermatozoa, Sertoli cells and myoid cells; cells of the ovary including ova, oogonia, oocytes, granulosa cells, theca cells (internal and external), germinal epithelial cells and follicle cells (primordial, vesicular, mature and atretic).

Also included are muscle cells such as myofibrils, intrafusal fibers and extrafusal fibers; skeletal system cells such as osteoblasts, osteocytes, osteoclasts and their progenitor cells.

Circulatory system cells are also included such cells as heart cells (myocardial cells); cells of the blood and lymph including erythropoietin-sensitive stem cells, erythrocytes, leukocytes (such as eosinophils, basophils and neutrophils (granular cells) and lymphocytes and monocytes (agranular cells)), thrombocytes, tissue macrophages (histiocytes), organ-specific phagocytes (such as Kupffer cells, alveolar macrophages and microglia), B-lymphocytes, T-lymphocytes (such as cytotoxic T cells, helperT cells and suppressorT cells), megaloblasts, monoblasts, myeloblasts, lymphoblasts, proerythroblasts, megakaryoblasts, promonocytes, promyelocytes, prolymphocytes, early normoblasts, megakaryocytes, intermediate normoblasts, metamyelocytes (such as juvenile metamyelocytes, segmented metamyelocytes and polymorphonuclear granulocytes), late normoblasts, reticulocytes and bone marrow cells.

Respiratory system cells are also included such as capillary endothelial cells and alveolar cells; as are urinary system cells such as nephrons, capillary endothelial cells, granular cells, tubule endothelial cells and podocytes; digestive system such as simple columnar epithelial cells, mucosal cells, acinar cells, parietal cells, chief cells, zymogen cells, peptic cells, enterochromaffin cells, goblet cells, Argentaffen cells and G cells; and sensory cells such as auditory system cells (hair cells); olfactory system cells such as olfactory receptor cells and columnar epithelial cells; equilibrium/vestibular apparatus cells including hair cells and supporting cells; visual system cells including pigment cells, epithelial cells, photoreceptor neurons (rods and cones), ganglion cells, amacrine cells, bipolar cells and horizontal cells are also included.

Additionally, mesenchymal cells, stromal cells, hair cells/follicles, adipose (fat) cells, cells of simple epithelial tissues (squamous epithelium, cuboidal epithelium, columnar epithelium, ciliated columnar epithelium and pseudostratified ciliated columnar epithelium), cells of stratified epithelial tissues (stratified squamous epithelium (keratinized and non-keratinized), stratified cuboidal epithelium and transitional epithelium), goblet cells, endothelial cells of the mesentery, endothelial cells of the small intestine, endothelial cells of the large intestine, endothelial cells of the vasculature capillaries, endothelial cells of the microvasculature, endothelial cells of the arteries, endothelial cells of the arterioles, endothelial cells of the veins, endothelial cells of the venules, etc.;cells of the connective tissue include chondrocytes, adipose cells, periosteal cells, endosteal cells, odontoblasts, osteoblasts, osteoclasts and osteocytes; endothelial cells, hepatocytes, keratinocytes and basal keratinocytes, muscle cells, cells of the central and peripheral nervous systems, prostate cells, and lung cells, cells in the lung, breast, pancreas, stomach, small intestine, and large intestine; epithelial cells such as sebocytes, hair follicles, hepatocytes, type II pneumocytes, mucin-producing goblet cells, and other epithelial cells and their progenitors of the skin, lung, liver, and gastrointestinal tract may be used in the methods of the present invention, preferably the selection and screening methods.

The cells may be in any cell phase, either synchronous or not, including M, G1, S, and G2. In a preferred embodiment, cells that are replicating or proliferating are used. Alternatively, non-replicating cells may be used.

Finally, any of the above cell types may be engineered to exhibit a non-naturally-occurring phenotype to be modified, for example, b, non-constitutive expression of a reporter gene operably associated with a regulatory pathway of interest.

### EXAMPLE 13

### Uses of Intracellular Immunoglobulins and Fragments

13.1 Intracellular Immunoglobulins. The hallmark of a malignant cell is uncontrolled proliferation. This phenotype is acquired through the accumulation of gene mutations, the majority of which promote passage through the cell cycle. Cancer cells ignore growth regulatory signals and remain committed to cell division. Classic oncogenes, such as ras, lead to inappropriate transition from G1 to S phase of the cell cycle, mimicking proliferative extracellular signals. Cell cycle checkpoint controls ensure faithful replication and segregation of the genome. The loss of cell cycle checkpoint control results in genomic instability, greatly accelerating the accumulation of mutations which drive malignant transformation. Hence, checkpoint regulators, such as p53 and ATM (ataxia telangiectasia mutated), also function as tumor suppressors. Thus, modulating cell cycle checkpoint pathways with therapeutic agents such as intracellular immunoglobulin molecules, or fragments thereof could exploit the differences between normal and tumor cells, both improving the selectivity of radio- and chemotherapy, and leading to novel cancer treatments.

For therapeutic use, the intracellular immunoglobulin or fragment thereof cassette is delivered to the cell by any of the known means. One preferred delivery system is described in U.S. patent application Ser. No. 08/199,070 by Marasco filed Feb. 22, 1994, which is incorporated herein by reference. This discloses the use of a fusion protein comprising a target moiety and a binding moiety. The target moiety brings the vector to the cell, while the binding moiety carries the antibody cassette. Other methods include, for example, Miller, A. D., Nature 357:455-460 (1992); Anderson, W. F., Science 256:808-813 (1992); Wu, et al., J. of Biol. Chem. 263:14621-14624 (1988). For example, a cassette containing an Ig polynucleotide can be targeted to a particular cell by a number of techniques.

Using intracellular immunoglobulin molecules, or fragments thereof (intrabodies) identified by the methods of the invention, one can treat mammals, preferably humans, suffering from an ailment or condition caused by the expression or overexpression of specific antigens, such as proteins. One can use intrabodies to treat viral infection, metabolic diseases, immunological diseases, etc. Individuals infected by viral diseases such as HIV, HTLV-1, HTLV-2, and herpes can be treated. Similarly, individuals having malignant tumors or susceptible to malignant cellular transformation caused by a high level of a protein or proteins, an altered protein or proteins or a combination thereof can be treated. For example, one can target at least one of the antigens with an antibody that will specifically bind to such antigen. One delivers an effective amount of a polynucleotide capable of expressing the antibody under conditions which will permit its intracellular expression to cells susceptible to expression of the undesired target antigen. This method can be used as a prophylactic treatment to prevent or make it more difficult for such cells to be adversely effected by the undesired antigen, for example, by preventing processing of the protein, interaction by the undesired protein with other proteins, integration by the virus into the host cell, etc. Where a number of targets exist, one preferred target is proteins that are processed by the endoplasmic reticulum. Intracellular delivery of any of the antibody polynucleotides can be accomplished by using gene therapy techniques.

Additional gene therapy applications for intrabodies are disclosed in Marasco, Gene Therapy 4:11-15 (1997).

13.2 Secreted or Membrane-Bound. Intracellular immunoglobulin molecules, or fragments thereof, isolated according to the methods of the invention may be cloned into other antibody frameworks, either full-length or fragments, and synthesized for further characterization or use, such a therapeutic use or use in research. Alternatively, they may be synthesized intracellularly, without having cloned them into another framework, for further characterization or use. The new antibody framework may allow secretion, as described herein. The immunoglobulins or immunoglobulin fragments may be “contacted” with antigen by a method which will allow an antigen which specifically recognizes a CDR of an immunoglobulin molecule to bind to the CDR, and which further allows detection of the antigen-antibody interaction. Such methods include, but are not limited to, immunoblots, ELISA assays, RIA assays, RAST assays, and immunofluorescence assays. Alternatively, the conditioned medium is subjected to a functional assay for specific antibodies. Examples of such assays include, but are not limited to, virus neutralization assays (for antibodies directed to specific viruses), bacterial opsonization/phagocytosis assays (for antibodies directed to specific bacteria), antibody-dependent cellular cytotoxicity (ADCC) assays, assays to detect inhibition or facilitation of certain cellular functions, assays to detect IgE-mediated histamine release from mast cells, hemagglutination assays, and hemagglutination inhibition assays. Such assays will allow detection of antigen-specific antibodies with desired functional characteristics.

Once a polynucleotide encoding an intracellular immunoglobulin or immunoglobulin fragment has been isolated, the CDR may be cloned into an antibody framework for synthesis. For example, the CDR may be cloned into an antibody framework which includes a signal peptide, thus resulting in a secreted antibody containing the CDR from the intracellular antibody. By “signal peptide” is meant a polypeptide sequence which, for example, directs transport of nascent immunoglobulin polypeptide subunit to the surface of the host cells. Signal peptides are also referred to in the art as “signal sequences,” “leader sequences,” “secretory signal peptides,” or “secretory signal sequences.” Signal peptides are normally expressed as part of a complete or “immature” polypeptide, and are normally situated at the N-terminus. The common structure of signal peptides from various proteins is commonly described as a positively charged n-region, followed by a hydrophobic h-region and a neutral but polar c-region. In many instances the amino acids comprising the signal peptide are cleaved off the protein once its final destination has been reached, to produce a “mature” form of the polypeptide. The cleavage is catalyzed by enzymes known as signal peptidases. The (−3, −1)-rule states that the residues at positions −3 and −1 (relative to the cleavage site) must be small and neutral for cleavage to occur correctly. See, e.g., McGeoch, Virus Res. 3:271-286 (1985), and von Heinje, Nucleic Acids Res. 14:4683-4690 (1986).

All cells, including host cells of the present invention, possess a constitutive secretory pathway, where proteins, including secreted immunoglobulin subunit polypeptides destined for export, are secreted from the cell. These proteins pass through the ER-Golgi processing pathway where modifications may occur. If no further signals are detected on the protein it is directed to the cells surface for secretion. Alternatively, immunoglobulin subunit polypeptides can end up as integral membrane components expressed on the surface of the host cells. Membrane-bound forms of immunoglobulin subunit polypeptides initially follow the same pathway as the secreted forms, passing through to the ER lumen, except that they are retained in the ER membrane by the presence of stop-transfer signals, or “transmembrane domains.” Transmembrane domains are hydrophobic stretches of about 20 amino acid residues that adopt an alpha-helical conformation as they transverse the membrane. Membrane embedded proteins are anchored in the phospholipid bilayer of the plasma membrane. As with secreted proteins, the N-terminal region of transmembrane proteins have a signal peptide that passes through the membrane and is cleaved upon exiting into the lumen of the ER. Transmembrane forms of immunoglobulin heavy chain polypeptides utilize the same signal peptide as the secreted forms.

A signal peptide of the present invention may be either a naturally-occurring immunoglobulin signal peptide, i.e., encoded by a sequence which is part of a naturally occurring heavy or light chain transcript, or a functional derivative of that sequence that retains the ability to direct the secretion of the immunoglobulin subunit polypeptide that is operably associated with it. Alternatively, a heterologous signal peptide, or a functional derivative thereof, may be used. For example, a naturally-occurring immunoglobulin subunit polypeptide signal peptide may be substituted with the signal peptide of human tissue plasminogen activator or mouse β-glucuronidase.

According to this embodiment, the polynucleotides isolated from the library(ies), for example, are cloned into antibody frameworks containing a signal peptide sequence and introduced into suitable host cells. Suitable host cells are characterized by being capable of expressing immunoglobulin molecules attached to their surface.

Membrane bound forms of immunoglobulins are typically anchored to the surface of cells by a transmembrane domain which is made part of the heavy chain polypeptide through alternative transcription termination and splicing of the heavy chain messengerRNA. See, e.g., Roitt at page 9.10. By “transmembrane domain” “membrane spanning region,” or related terms, which are used interchangeably herein, is meant the portion of heavy chain polypeptide which is anchored into a cell membrane. Typical transmembrane domains comprise hydrophobic amino acids as discussed in more detail below. By “intracellular domain,” “cytoplasmic domain,” “cytosolic region,” or related terms, which are used interchangeably herein, is meant the portion of the polypeptide which is inside the cell, as opposed to those portions which are either anchored into the cell membrane or exposed on the surface of the cell. Membrane-bound forms of immunoglobulin heavy chain polypeptides typically comprise very short cytoplasmic domains of about three amino acids. A membrane-bound form of an immunoglobulin heavy chain polypeptide of the present invention preferably comprises the transmembrane and intracellular domains normally associated with that immunoglobulin heavy chain, e.g., the transmembrane and intracellular domains associated with μ and δ heavy chains in pre-B cells, or the transmembrane and intracellular domains associated with any of the immunoglobulin heavy chains in B-memory cells. However, it is also contemplated that heterologous transmembrane and intracellular domains could be associated with a given immunoglobulin heavy chain polypeptide, for example, the transmembrane and intracellular domains of a μ heavy chain could be associated with the extracellular portion of a γ heavy chain. Alternatively, transmembrane and/or cytoplasmic domains of an entirely heterologous polypeptide could be used, for example, the transmembrane and cytoplasmic domains of a major histocompatibility molecule, a cell surface receptor, a virus surface protein, chimeric domains, or synthetic domains.

### EXAMPLE 14

### Attenuation of Poxvirus Mediated Host Shut-off by Reversible Inhibitor of DNA Synthesis

As discussed herein, it si sometimes desired to use attenuated or defective virus to reduce cytopathic effects. Cytopathic effects during poxvirus infection might interfere with selection and identification of certain intrabodies. Such effects can be attenuated with a reversible inhibitor of DNA synthesis such as hydroxyurea (HU) (Pogo, B. G. and S. Dales, Biogenesis of vaccinia: separation of early stages from maturation by means of hydroxyurea. Virology, 1971. 43(1):144-51). HU inhibits both cell and viral DNA synthesis by depriving replication complexes of deoxyribonucleotide precursors (Hendricks, S. P. and C. K. Mathews, Differential effects of hydroxyurea upon deoxyribonucleoside triphosphate pools, analyzed with vaccinia virus ribonucleotide reductase. J Biol Chem, 1998. 273(45):29519-23). Inhibition of viral DNA replication blocks late viral RNA transcription while allowing transcription and translation of genes under the control of early vaccinia promoters (Nagaya, A., B. G. Pogo, and S. Dales, Biogenesis of vaccinia: separation of early stages from maturation by means of rifampicin. Virology, 1970. 40(4):1039-51). Thus, treatment with reversible inhibitor of DNA synthesis such as HU allows the detection of effects of intrabodies. Following appropriate incubation, HU inhibition can be reversed by washing the host cells so that the viral replication cycle continues and infectious recombinants can be recovered (Pogo, B. G. and S. Dales, Biogenesis of vaccinia: separation of early stages from maturation by means of hydroxyurea. Virology, 1971. 43(1):144-51).

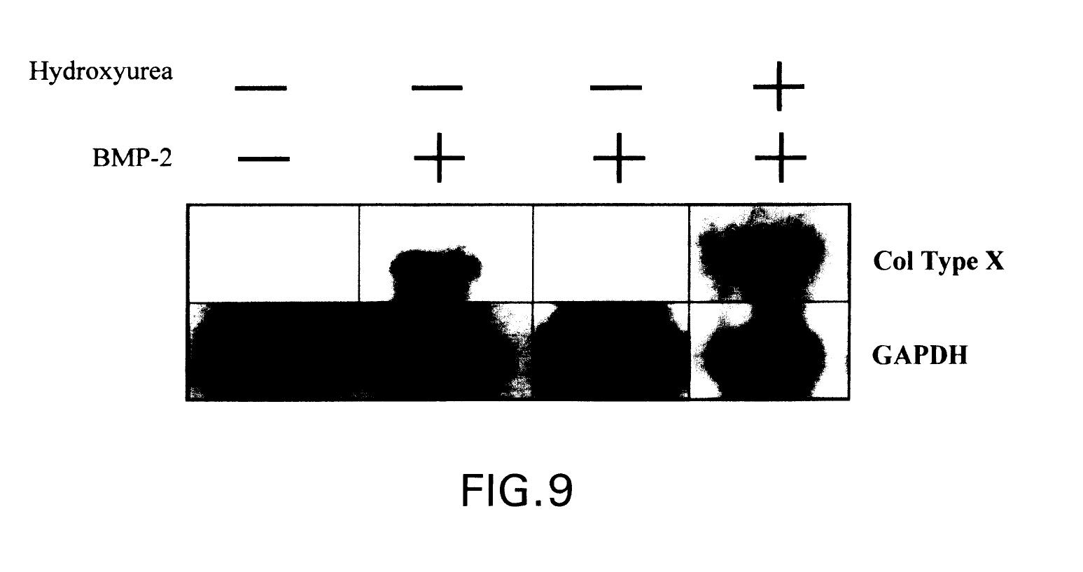

The results in FIG. 9 demonstrate that induction of type X collagen synthesis, a marker of chondrocyte differentiation, in C3H10T 1/2 progenitor cells treated with BMP-2 (Bone Morphogenetic Protein-2) is blocked by vaccinia infection but that its synthesis can be rescued by HU mediated inhibition of viral DNA synthesis. When HU is removed from cultures by washing with fresh medium, viral DNA synthesis and assembly of infectious particles proceeds rapidly so that infectious viral particles can be isolated as soon as 2 hrs post-wash.

C3H10T 1/2 cells were infected with WR vaccinia virus at MOI=1 and 1 hour later either medium or 400 ng/ml of BMP-2 in the presence or absence of 2 mM HU was added. After a further 21 hour incubation at 37° C., HU was removed by washing with fresh medium. The infectious cycle was allowed to continue for another 2 hours to allow for initiation of viral DNA replication and assembly of infectious particles. At 24 hours RNA was extracted from cells maintained under the 4 different culture conditions. Northern analysis was carried out using a type X collagen specific probe. The uninduced C3H10T1/2 cells have a mesenchymal progenitor cell phenotype and as such do not express type X collagen (first lane from left). Addition of BMP-2 to normal, uninfected C3H10T 1/2 cells induces differentiation into mature chondrocytes and expression of type X collagen (compare first and second lanes from left), whereas addition of BMP-2 to vaccinia infected C3H10T 1/2 cells fails to induce synthesis of type X collagen (third lane from left). In the presence of 2mM HU, BMP-2 induces type X collagen synthesis even in vaccinia virus infected C3H10T 1/2 cells (fourth lane from left).

This strategy for attenuating viral cytopathic effects is applicable to other cell types and to selection of intrabodies that regulate expression of other host genes.

### EXAMPLE 15

### Construction of Human Single-Chain-Fv (ScFv) Intrabody Libraries.

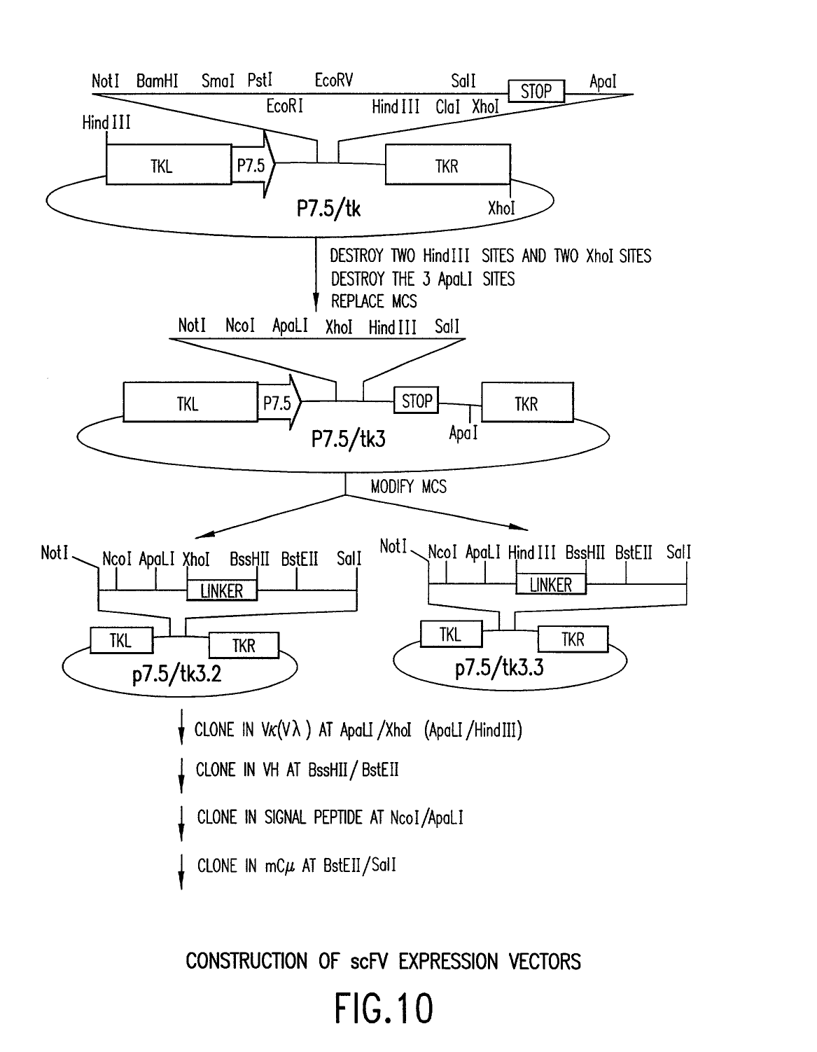

15.1 Human scFv expression vectors p7.5/tk3.2 and p7.5/tk3.3 are constructed by the following method, as illustrated in FIG. 10. Plasmid p7.5/tk3.1 is produced as described in Example 1 herein.

Plasmid p7.5/tk3.1 is converted to p7.5/tk3.2 by substituting the region between XhoI and SalI (i.e., nucleotides 30 to 51 of SEQ ID NO:81 [0299]) with the following cassette: XhoI-(nucleotides encoding amino acids 106-107 of Vκ)-(nucleotides encoding a 10 amino acid linker)-G-BssHII-ATGC-BstEII-(nucleotides encoding amino acids 111-113 of VH)-stop codon-SalI. This is accomplished by digesting p7.5/tk3.1 with XhoI and SalI, and inserting a cassette having the sequence 5′CTCGAGAT CAAAGAGGGT AAATCTTCCG GATCTGGTTC CGAAGGCGCG CATGCGGTCA CCGTCTCCTC ATGAGTCGAC 3′, referred to herein as SEQ ID NO:144. The linker between Vκ and VH will have a final size of 14 amino acids, with the last 4 amino acids contributed by the VH PCR products, inserted as described below. The sequence of the linker is 5′GAG GGT AAA TCT TCC GGA TCT GGT TCC GAA GGC GCG CAC TCC 3′ (SEQ ID NO:145), which encodes amino acids EGKSSGSGSEGAHS (SEQ ID NO 146).

Plasmid p7.5/tk3.1 is converted to p7.5/tk3.3 by substituting the region between HindIII and SalI (i.e., nucleotides 36 to 51 of SEQ ID NO:81 [0299]) with the following cassette: HindIII-(nucleotides encoding amino acid residues 105-107 of Vλ)-(nucleotides encoding a 10 amino acid linker)-G-BssHII-ATGC-BstEII-(nucleotides encoding amino acids 111-113 of VH)-stop codon-SalI. This is accomplished by digesting p7.5/tk3.1 with HindIII and SalI, and inserting a cassette having the sequence 5′AAGCTTACCG TCCTAGAGGG TAAATCTTCC GGATCTGGTTC CGAAGGCGCG CATGCGGTCA CCGTCTCCTC ATGAGTCGAC 3′ (SEQ ID NO:147). The linker between Vλ and VH will have a final size of 14 amino acids, with the last 4 amino acids contributed by the VH PCR products, inserted as described below. The sequence of the linker is 5′GAG GGT AAA TCT TCC GGA TCT GGT TCC GAA GGC GCG CAC TCC 3′ (SEQ ID NO:148), which encodes amino acids EGKSSGSGSEGAHS (SEQ ID NO:149).

15.2 Cytosolic Forms of scFv. Expression vectors encoding scFv polypeptides comprising human κ or λ immunoglobulin light chain variable regions, fused in frame with human heavy chain variable regions, are constructed as follows.

(a) Cytosolic VKVH scFv expression products are prepared as follows. Kappa light chain variable region (Vκ) PCR products (amino acids(−3) to(105)), produced as described in Example 1.3(b), using the primers listed in Table 3, are cloned into p7.5/tk3.2 between the ApaLI and XhoI sites. Because of the overlap between the κ light chain sequence and the restriction enzyme sites selected, this results in construction of a contiguous κ light chain in the same translational reading frame as the downstream linker. Heavy chain variable region (VH) PCR products (amino acids (−4) to(110)), produced as described in Example 1.3(a), using the primers listed in Table 3, are cloned between the BssHII and BstEII sites of p7.5/tk3.2 to form complete scFv open reading frames. The resulting products are cytosolic forms of Vκ-VH fusion proteins connected by a linker of 14 amino acids. The scFv is also preceded by 6 extra amino acids at the amino terminus encoded by the restriction sites and part of the Vκ signal peptide.

(b) Cytosolic VλVH scFv expression products are prepared as follows. Lambda light chain variable region (VL) PCR products (amino acids(−3) to(104)), produced as described in Example 1.3(c), using the primers listed in Table 3, are cloned into p7.5/tk3.3 between the ApaLI and HindIII sites. Because of the overlap between the λ light chain sequence and the restriction enzyme sites selected, this results in construction of a contiguous λ light chain in the same translational reading frame as the downstream linker. Heavy chain variable region (VH) PCR products (amino acids (−4) to(110)), produced as described in Example 1.3(a), using the primers listed in Table 3, are cloned between BssHII and BstEII sites of p7.5/tk3.3 to form complete scFv open reading frames. The resulting products are cytosolic forms of Vλ-VH fusion proteins connected by a linker of 14 amino acids. The scFv is also preceded by 6 extra amino acids at the amino terminus encoded by the restriction sites and part of the Vλ signal peptide.

15.3 Expression of scFv in other intracellular organelles

The cytosolic scFv expression vectors described in section 14.2 serve as the prototype vectors for cloning in other organelle-specific localization signals to target scFv to specific subcellular compartments (scFv intrabodies). These localization signals are inserted either in the N-terninus of scFv between NcoI and ApaLI or in the C-terminus between BstEII and SalI. Localization signals include but are not limited to those listed in Table 4, herein.

### EXAMPLE 16

### Construction of Camelized Human Single-Domain Intrabody Libraries

Camelid species use only heavy chains to generate antibodies, which are termed heavy chain antibodies. The poxvirus expression system is amendable to generate intracellular human single-domain libraries, wherein the human V_{H} domain is “camelized,” i.e., is altered to resemble the V_{H}H domain of a camelid antibody, which can then be selected based on either functional assays or Ig-crosslinking/binding. Human V_{H} genes are camelized by standard mutagenesis methods to more closely resemble camelid V_{H}H genes. For example, human V_{H}3 genes, produced using the methods described in Example 1 using appropriate primer pairs selected from Table 3, is camelized by substituting G44 with E, L45 with R, and W47 with G or I. See, e.g., Riechmann, L., and Muyldermans, S. J. Immunol. Meth. 231:25-38. To generate an intracellular single-domain intrabody library, cassettes encoding camelized human V_{H} genes are cloned into pVHEc, produced as described in Example 1, to be expressed in-frame between the BssHII and BstEII sites. Amino acid residues in the three CDR regions of the camelized human V_{H} genes are subjected to extensive randomization, and the resulting libraries are selected in poxviruses as described herein. Finally, to generate a single-domain antibody targeted to other intracellular organelles (a camelized intrabody), localization signals, including but not limited to those listed in Table 4, are inserted either in the N-terminus between NcoI and ApaLI or in the C-terminus between BstEII and SalI.

The present invention is not to be limited in scope by the specific embodiments described which are intended as single illustrations of individual aspects of the invention, and any constructs, viruses or enzymes which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims.

All publications and patent applications mentioned in this specification are herein incorporated by reference to the same extent as if each individual publication or patent application was specifically and individually indicated to be incorporated by reference. The disclosure and claims of U.S. application Ser. No. 08/935,377, filed Sep. 22,1997, and U.S. application Ser. No. 60/192,586, filed Mar. 28, 2000, are herein incorporated by reference.

What is claimed is:

1. A method of selecting polynucleotides which encode an intracellular immunoglobulin molecule, or fragment thereof, whose expression induces a modified phenotype in a eukaryotic host cell, comprising:
(a) providing a population of eukaryotic host cells capable of expressing said intracellular immunoglobulin molecule, or fragment thereof, wherein individual host cells of said population can be induced to exhibit a predetermined modified phenotype;
(b) introducing into said population of host cells a first library of polynucleotides encoding, through operable association with a transcriptional control region, a plurality of first intracellular immunoglobulin subunit polypeptides, each comprising a first immunoglobulin variable region selected from the group consisting of a heavy chain variable region and a light chain variable region;
(c) introducing into said population of host cells a second library of polynucleotides encoding, through operable association with a transcriptional control region, a plurality of second intracellular immunoglobulin subunit polypeptides, each comprising a second immunoglobulin variable region selected from the group consisting of a heavy chain variable region and a light chain variable region, wherein said second immunoglobulin variable region is not the same as said first immunoglobulin variable region, and wherein said second intracellular immunoglobulin subunit polypeptides combine with said first intracellular immunoglobulin subunit polypeptides to form a plurality of intracellular immunoglobulin molecules, or fragments thereof;
(d) permitting expression of said plurality of intracellular immunoglobulin molecules, or fragments thereof in said population of host cells under conditions wherein said modified phenotype can be detected; and
(e) recovering polynucleotides of said first library from those individual host cells which exhibit said modified phenotype.

2. The method of claim 1, further comprising:
(f) providing a population of eukaryotic host cells capable of expressing said intracellular immunoglobulin molecule, or fragment thereof, wherein individual host cells of said population can be induced to exhibit a predetermined modified phenotype;
(g) introducing said polynucleotides recovered from said first library into said population of host cells;
(h) introducing into said population of host cells said second library of polynucleotides;
(i) permitting expression of said plurality of intracellular immunoglobulin molecules, or fragments thereof in said population of host cells under conditions wherein said modified phenotype can be detected; and
(j) recovering polynucleotides of said first library from those individual host cells which exhibit said modified phenotype.

3. The method of claim 2, further comprising repeating steps (f)-(j) one or more times, thereby enriching for polynucleotides of said first library which encode a first intracellular immunoglobulin subunit polypeptide whose expression, as part of an intracellular immunoglobulin molecule, or fragment thereof, induces said modified phenotype.

4. The method claim 1, further comprising isolating those polynucleotides recovered from said first library.

5. The method of claim 4, further comprising:
(k) providing a population of eukaryotic host cells capable of expressing said intracellular immunoglobulin molecule, or fragment thereof, wherein individual host cells of said population can be induced to exhibit a predetermined modified phenotype;
(l) introducing into said population of host cells said second library of polynucleotides;
(m) introducing into said population host cells said first polynucleotides isolated from said first library, wherein the intracellular immunoglobulin subunit polypeptides encoded by said isolated first polynucleotides combine with said second intracellular immunoglobulin subunit polypeptides encoded by said second library of polynucleotides to form a plurality of intracellular immunoglobulin molecules, or fragments thereof;
(n) permitting expression of said plurality of intracellular immunoglobulin molecules, or fragments thereof in said population of host cells under conditions wherein said modified phenotype can be detected; and
(o) recovering polynucleotides of said second library from those individual host cells which exhibit said modified phenotype.

6. The method of claim 5, further comprising:
(p) providing a population of eukaryotic host cells capable of expressing said intracellular immunoglobulin molecule, or fragment thereof, wherein individual host cells of said population can be induced to exhibit a predetermined modified phenotype;
(q) introducing said polynucleotides recovered from said second library into said population of host cells;
(r) introducing into said population of host cells said first polynucleotides isolated from said first library, wherein the intracellular immunoglobulin subunit polypeptides encoded by said isolated first polynucleotides combine with the second intracellular immunoglobulin subunit polypeptides encoded by said polynucleotides recovered from said second library, to form a plurality of intracellular immunoglobulin molecules, or fragments thereof;
(s) permitting expression of said plurality of intracellular immunoglobulin molecules, or fragments thereof in said population of host cells under conditions wherein said modified phenotype can be detected; and
(t) recovering polynucleotides of said second library from those individual host cells which exhibit said modified phenotype.

7. The method of claim 6, further comprising repeating steps (p)-(t) one or more times, thereby enriching for polynucleotides of said second library which encode a second intracellular immunoglobulin subunit polypeptide whose expression, as part of an intracellular immunoglobulin molecule, or fragment thereof, induces said modified phenotype.

8. The method of claim 5, further comprising isolating those polynucleotides recovered from said second library.

9. The method of claim 1, wherein said intracellular immunoglobulin molecule, or fragment thereof is derived from a human immunoglobulin molecule.

10. The method of claim 1, wherein said first intracellular immunoglobulin subunit polypeptide comprises a heavy chain variable region.

11. The method of claim 10, wherein said first intracellular immunoglobulin subunit polypeptide further comprises a heavy chain constant region, or fragment thereof.

12. The method of claim 11, where in said second intracellular immunoglobulin subunit polypeptide further comprises a light chain constant region, or fragment thereof.

13. The method of claim 1, wherein said first intracellular immunoglobulin subunit polypeptide comprises a light chain variable region.

14. The method of claim 13, wherein said light chain variable region is a kappa variable region.

15. The method of claim 13, wherein said light chain variable region is a lambda variable region.

16. The method of claim 13, wherein said first intracellular immunoglobulin subunit polypeptide further comprises a light chain constant region, or fragment thereof.

17. The method of claim 16, wherein said second intracellular immunoglobulin subunit polypeptide further comprises a heavy chain constant region, or fragment thereof.

18. The method of claim 1, wherein said first library of polynucleotides is introduced into said population of eukaryotic host cells by means of a eukaryotic virus vector.

19. The method of claim 1, wherein said second library of polynucleotides is introduced into said population of eukaryotic host cells by means of a eukaryotic virus vector.

20. The method of claim 5, wherein said first polynucleotides isolated from said first library are introduced into said population of eukaryotic host cells by means of a eukaryotic virus vector.

21. The method of claim 1, wherein said second library of polynucleotides is introduced into said population of eukaryotic host cells by means of a plasmid vector.

22. The method of claim 18, wherein said population of eukaryotic host cells are infected with said first library at a multiplicity of infection ranging from about 1 to about 10, and wherein said second library is introduced under conditions which allow up to 20 polynucleotides of said second library to be taken up by each infected host cell.

23. The method of claim 5, wherein said first polynucleotides isolated from said first library are introduced into said population of eukaryotic host cells by means of a plasmid vector.

24. The method of claim 18, wherein said eukaryotic virus vector is an animal virus vector.

25. The method of claim 19, wherein said eukaryotic virus vector is an animal virus vector.

26. The method of claim 24, wherein said vector is capable of producing infectious virus particles in mammalian cells.

27. The method of claim 26, wherein the naturally-occurring genome of said vector is DNA.

28. The method of claim 26, wherein the naturally-occurring genome of said vector is RNA.

29. The method of claim 27, wherein the naturally-occurring genome of said vector is linear, double-stranded DNA.

30. The method of claim 29, wherein said vector is selected from the group consisting of an adenovirus vector, a herpesvirus vector and a poxvirus vector.

31. The method of claim 30, wherein said vector is a poxvirus vector.

32. The method of claim 31, wherein said poxvirus vector is selected from the group consisting of an orthopoxvirus vector, an avipoxvirus vector, a capripoxvirus vector, a leporipoxvirus vector, an entomopoxvirus vector, and a suipoxvirus vector.

33. The method of claim 32, wherein said poxvirus vector is an orthopoxvirus vector selected from the group consisting of a vaccinia virus vector and a raccoon poxvirus vector.

34. The method of claim 33, wherein said poxvirus vector is a vaccinia virus vector.

35. The method of claim 34, wherein said host cells are permissive for the production of infectious virus particles of said vaccinia virus vector.

36. The method of claim 34, wherein said vaccinia virus vector is attenuated.

37. The method of claim 36, wherein said vaccinia virus vector is deficient in D4R synthesis.

38. The method of claim 31, wherein said transcriptional control region of said first library of polynucleotides functions in the cytoplasm of a poxvirus-infected cell.

39. The method of claim 21, wherein said plasmid vector directs synthesis of said second immunoglobulin subunit in the cytoplasm of a poxvirus-infected cell through operable association with a poxvirus-derived transcriptional control region.

40. The method of claim 38, wherein said transcriptional control region comprises a promoter.

41. The method of claim 40, wherein said promoter is constitutive.

42. The method of claim 41, wherein said promoter is a vaccinia virus p7.5 promoter.

43. The method of claim 42, wherein said promoter is a synthetic early/late promoter.

44. The method of claim 40, wherein said promoter is a T7 phage promoter active in cells in which T7 RNA polymerase is expressed.

45. The method of claim 38, wherein said transcriptional control region comprises a transcriptional termination region.

46. The method of claim 18, wherein said first library of polynucleotides is constructed by a method comprising:
(a) Providing a population of host cells permissive for the production of infectious viral particles of said eukaryotic virus vector;
(b) cleaving an isolated linear DNA fragment comprising the genome of said eukaryotic virus vector to produce a first viral fragment and a second viral fragment, wherein said first fragment is nonhomologous with said second fragment;
(c) providing a population of transfer plasmids comprising polynucleotides encoding said plurality of first intracellular immunoglobulin subunit polypeptides through operable association with a transcription control region, wherein each of said polynucleotides is flanked by a 5′ flanking region and a 3′ flanking region, wherein said 5′ flanking region is homologous to said first viral fragment and said 3′ flanking region is homologous to said second viral fragment;
(d) introducing said transfer plasmids and said first and second viral fragments into said population of host cells under conditions wherein each of said transfer plasmids, said first viral fragment, and said second viral fragment undergo in vivo homologous recombination, thereby producing a population of viable modified virus genomes, each comprising a polynucleotide which encodes a first intracellular immunoglobulin subunit polypeptide; and
(e) recovering said population of modified virus genomes.

47. The method of claim 19, wherein said second library of polynucleotides is constructed by a method comprising:
(a) Providing a population of host cells permissive for the production of infectious viral particles of said eukaryotic virus vector;
(b) cleaving an isolated linear DNA fragment comprising the genome of said eukaryotic virus vector to produce a first viral fragment and a second viral fragment, wherein said first fragment is nonhomologous with said second fragment;
(c) providing a population of transfer plasmids comprising polynucleotides encoding said plurality of second intracellular immunoglobulin subunit polypeptides through operable association with a transcription control region, wherein each of said polynucleotides is flanked by a 5 ′ flanking region and a 3′ flanking region, wherein said 5′ flanking region is homologous to said first viral fragment and said 3′ flanking region is homologous to said second viral fragment;
(d) introducing said transfer plasmids and said first and second viral fragments into said population of host cells under conditions wherein each of said transfer plasmids, said first viral fragment, and said second viral fragment undergo in vivo homologous recombination, thereby producing a population of viable modified virus genomes, each comprising a polynucleotide which encodes a second intracellular immunoglobulin subunit polypeptide; and
(e) recovering said population of modified virus genomes.

48. The method of claim 1, wherein said population of eukaryotic host cells is adherent to a solid support and wherein said modified phenotype is nonadherence.

49. The method claim 48, wherein said nonadherence is due to an inhibition of an essential function by said intracellular immunoglobulin molecule, or fragment thereof.

50. The method of claim 48, wherein said population of eukaryotic host cells each comprise a suicide gene in operable association with a non-constitutive promoter, and wherein said nonadherence is due to expression of said suicide gene from said promoter.

51. The method of claim 50, wherein said non-constitutive promoter is selected from the group consisting of: a differentiation-induced promoter, a cell type-restricted promoter, a tissue-restricted promoter, a temporally-regulated promoter, a spatially-regulated promoter, a proliferation-induced promoter, and a cell-cycle specific promoter.

52. The method of claim 48, wherein said population of eukaryotic host cells is not yeast cells, wherein each of said host cells further comprises a suicide gene in operable association with a regulatory region as part of a two-hybrid system, and wherein said nonadherence is due to expression of said suicide gene from said regulatory region.

53. The method of claim 1, wherein said population of eukaryotic host cells each comprise a polynucleotide encoding a cell surface antigen in operable association with a non-constitutive promoter, and wherein said modified phenotype is expression of said cell surface antigen.

54. The method of claim 53, wherein expression of said cell surface antigen is detected by binding of an antibody specific for said cell surface antigen.

55. The method of claim 1, wherein said population of eukaryotic host cells each comprise a polynucleotide encoding a cell surface antigen in operable association with a non-constitutive promoter, and wherein said modified phenotype is reduced expression of said cell surface antigen.

56. The method of claim 55, wherein reduced expression of said cell surface antigen is detected by a reduction in binding of an antibody specific for said cell surface antigen.

57. The method of claim 1, wherein said modified phenotype is altered susceptibility to an infectious agent.

58. The method of claim 1, wherein said modified phenotype is altered drug sensitivity.

59. The method of claim 1, wherein each of said first library of polynucleotides further comprise a heterologous polynucleotide, wherein said heterologous polynucleotide is common to each polynucleotide in said first library.

60. The method of claim 59, wherein said heterologous polynucleotide encodes a heterologous polypeptide fused to each of said first intracellular immunoglobulin subunit polypeptides.

61. The method of claim 60, wherein said heterologous polypeptide is a targeting sequence.

62. The method of claim 61, wherein said targeting sequence is capable of localizing said intracellular immunoglobulin molecule, or fragment thereof, to a subcellular location selected from the group consisting of a golgi, an endoplasmic reticulum, a nucleus, a nucleoli, a nuclear membrane, a mitochondria, a chloroplast, a secretory vesicle, a lysosome, and a cellular membrane.

63. The method of claim 60, wherein said heterologous polypeptide is an epitope tag.

64. The method of claim 63, wherein said epitope tag is selected from the group consisting of a myc epitope, a BSP biotinylation target sequence of the bacterial enzyme BirA, a tag derived from a protein of the influenza virus, β-galactosidase, glutathione-S-transferase (GST), or a detectable fragment of any of said epitope tags.

65. The method of claim 60, wherein said heterologous polypeptide is a 6-His tag.

66. A kit for the identification of an intracellular immunoglobulin molecule, or fragment thereof, whose expression results in a modified phenotype in a eukaryotic host cell, comprising:
(a) a first library of polynucleotides encoding, through operable association with a transcriptional control region, a plurality of first intracellular immunoglobulin subunit polypeptides, each comprising a first immunoglobulin variable region selected from the group consisting of a heavy chain variable region and a light chain variable region, wherein said first library is constructed in a eukaryotic virus vector;
(b) a second library of polynucleotides encoding, through operable association with a transcriptional control region, a plurality of second intracellular immunoglobulin subunit polypeptides, each comprising a second immunoglobulin variable region selected from the group consisting of a heavy chain variable region and a light chain variable region, wherein said second immunoglobulin variable region is not the same as said first immunoglobulin variable region, wherein said second intracellular immunoglobulin subunit polypeptides combine with said first intracellular immunoglobulin subunit polypeptides to form a plurality of intracellular immunoglobulin molecules, or fragments thereof, and wherein said second library is constructed in a eukaryotic virus vector; and
(c) a population of eukaryotic host cells capable of expressing said intracellular immunoglobulin molecule or fragment thereof, wherein individual host cells of said population can be induced to exhibit a predetermined modified phenotype;
wherein said first and second libraries are provided both as infectious virus particles and as inactivated virus particles, and wherein said inactivated virus particles are taken up by said host cells, which said first and second intracellular immunoglobulin subunit polypeptides, but do not undergo virus replication; and
wherein polynucleotides encoding said first and second intracellular immunoglobulin subunit polypeptides are recoverable from individual host cells which exhibit said modified phenotype.

67. An intracellular immunoglobulin, or fragment thereof, produced by the method of claim 1.

68. A composition comprising the intracellular immunoglobulin, or fragment thereof of claim 67, and a pharmaceutically acceptable carrier.

69. A method of selecting polynucleotides which encode a single-chain intracellular immunoglobulin whose expression induces a modified phenotype in a eukaryotic host cell, comprising:
(a) providing a population of eukaryotic host cells capable of expressing said single-chain intracellular immunoglobulin, wherein individual host cells of said population can be induced to exhibit a predetermined modified phenotype;
(b) introducing into said host cells a library of polynucleotides encoding, through operable association with a transcriptional control region, a plurality of single-chain intracellular immunoglobulins, each comprising a heavy chain variable region;
(c) permitting expression of said plurality of single-chain intracellular immunoglobulins in said host cells under conditions wherein said modified phenotype can be detected; and
(d) recovering polynucleotides of said library from those individual host cells which exhibit said modified phenotype.

70. The method of claim 69, wherein said heavy chain variable region is camelized.

71. The method of claim 69, wherein each of said plurality of single-chain intracellular immunoglobulins further comprises a light chain variable region.

72. The method of claim 69, further comprising:
(e) providing a population of eukaryotic host cells capable of expressing said single-chain intracellular immunoglobulin, wherein individual host cells of said population can be induced to exhibit a predetermined modified phenotype;
(f) introducing the polynucleotides recovered in (d) into said host cells;
(g) permitting expression of said single-chain intracellular immunoglobulins encoded by said recovered polynucleotides in said host cells under conditions wherein said modified phenotype can be detected; and
(h) recovering polynucleotides of said library from those individual host cells which exhibit said modified phenotype.

73. The method of claim 72, further comprising repeating steps (e)-(h) one or more times, thereby enriching for polynucleotides of said library which encode a single-chain intracellular immunoglobulin whose expression induces said modified phenotype.

74. The method of claim 69, further comprising isolating those polynucleotides recovered from said library.

75. The method of claim 71, wherein said heavy chain variable region and said light chain variable region are directly linked.

76. The method of claim 71, wherein each of said plurality of single-chain intracellular immunoglobulins further comprises a peptide linker which joins said heavy chain variable region and said light chain variable region.

77. The method of claim 69, wherein each of said plurality of single-chain intracellular immunoglobulins further comprises a heavy chain constant region domain.

78. The method of claim 7 1, wherein each of said plurality of single-chain intracellular immunoglobulins further comprises a heavy chain constant region domain.

79. The method of claim 71, wherein each of said plurality of single-chain intracellular immunoglobulins further comprises a light chain constant region domain.

80. The method of claim 69, wherein each of said plurality of single-chain intracellular immunoglobulins further comprises a leucine zipper.

81. A k it for the identification of a single-chain intracellular immunoglobulin whose expression induces a modified phenotype in a eukaryotic host cell, comprising:
(a) a library of polynucleotides encoding, through operable association with a transcriptional control region, a plurality of single-chain intracellular immunoglobulins, each comprising a heavy chain variable region, wherein said library is constructed in a eukaryotic virus vector; and
(b) a population of eukaryotic host cells capable of expressing said single-chain intracellular immunoglobulin, wherein individual host cells of said population can be induced to exhibit a predetermined modified phenotype;
wherein a polynucleotide encoding said single-chain intracellular immunoglobulin is recoverable from individual host cells which exhibit said modified phenotype.

82. The kit of claim 80, wherein each of said plurality of single-chain intracellular immunoglobulins further comprises a light chain variable region.

83. A single-chain intracellular immunoglobulin produced by the method of claim 69.

84. A composition comprising the single-chain intracellular immunoglobulin of claim 83, and a pharmaceutically acceptable carrier.


SEQUENCE LISTING (154 sequences)

SEQ ID NO: 1 - protein, 15 aa, Artificial Sequence
  Linker
GGGGSGGGGSGGGGS

SEQ ID NO: 2 - protein, 15 aa, Artificial Sequence
  Linker
ESGRSGGGGSGGGGS

SEQ ID NO: 3 - protein, 14 aa, Artificial Sequence
  Linker
EGKSSGSGSESKST

SEQ ID NO: 4 - protein, 15 aa, Artificial Sequence
  Linker
EGKSSGSGSESKSTQ

SEQ ID NO: 5 - protein, 14 aa, Artificial Sequence
  Linker
EGKSSGSGSESKVD

SEQ ID NO: 6 - protein, 14 aa, Artificial Sequence
  Linker
GSTSGSGKSSEGKG

SEQ ID NO: 7 - protein, 18 aa, Artificial Sequence
  Linker
KESGSVSSEQLAQFRSLD

SEQ ID NO: 8 - protein, 16 aa, Artificial Sequence
  Linker
ESGSVSSEELAFRSLD

SEQ ID NO: 9 - DNA, 150 bp, Artificial Sequence
  p7.5/ATG3/tk vector
ggccaaaaattgaaaaactagatctatttattgcacgcggccgccatgacgtggatcccccgggctgcaggaattcgatatcaagcttatcgataccgtcgacctcgagggggggcctaactaactaattttgtttttgtgggcccggcc

SEQ ID NO: 10 - protein, 7 aa, Artificial Sequence
  Signal sequence
PKKKRKV

SEQ ID NO: 11 - protein, 6 aa, Artificial Sequence
  signal sequence
ARRRRP

SEQ ID NO: 12 - protein, 10 aa, Artificial Sequence
  signal sequence
EEVQRKRQKL

SEQ ID NO: 13 - protein, 9 aa, Artificial Sequence
  signal sequence
EEKRKRTYE

SEQ ID NO: 14 - protein, 20 aa, Artificial Sequence
  signal sequence
AVKRPAATKKAGQAKKKKLD

SEQ ID NO: 15 - protein, 31 aa, Artificial Sequence
  signal sequence
MASPLTRFLSLNLLLLGESILGSGEAKPQAP

SEQ ID NO: 16 - protein, 21 aa, Artificial Sequence
  signal sequence
MSSFGYRTLTVALFTLICCPG

SEQ ID NO: 17 - protein, 14 aa, Artificial Sequence
  myristylation sequence
MGSSKSKPKDPSQR

SEQ ID NO: 18 - protein, 51 aa, Artificial Sequence
  transmembrane domain
PQRPEDCRPRGSVKGTGLDFACDIYIWAPLAGICVALLLSLIITLICYHSR

SEQ ID NO: 19 - protein, 33 aa, Artificial Sequence
  transmembrane domain
MVIIVTVVSVLLSLFVTSVLLCFIFGQHLRQQR

SEQ ID NO: 20 - protein, 37 aa, Artificial Sequence
  anchor sequence
PNKGSGTTSGTTRLLSGHTCFTLTGLLGTLVTMGLLT

SEQ ID NO: 21 - protein, 26 aa, Artificial Sequence
  palmitoylation sequence
LLQRLFSRQDCCGNCSDSEEELPTRL

SEQ ID NO: 22 - protein, 20 aa, Artificial Sequence
  palmitoylation sequence
KQFRNCMLTSLCCGKNPLGD

SEQ ID NO: 23 - protein, 19 aa, Artificial Sequence
  palmitoylation sequence
LNPPDESGPGCMSCKCVLS

SEQ ID NO: 24 - protein, 5 aa, Artificial Sequence
  membrane sequence
KFERQ

SEQ ID NO: 25 - protein, 36 aa, Artificial Sequence
  membrane sequence
MLIPIAGFFALAGLVLIVLIAYLIGRKRSHAGYQTI

SEQ ID NO: 26 - protein, 35 aa, Artificial Sequence
  membrane sequence
LVPIAVGAALAGVLILVLLAYFIGLKHHHAGYEQF

SEQ ID NO: 27 - protein, 27 aa, Artificial Sequence
  targeting sequence
MLRTSSLFTRRVQPSLFSRNILRLQST

SEQ ID NO: 28 - protein, 25 aa, Artificial Sequence
  targeting sequence
MLSLRQSIRFFKPATRTLCSSRYLL

SEQ ID NO: 29 - protein, 63 aa, Artificial Sequence
  targeting sequence
MFSMLSKRWAQRTLSKSFYSTATGAASKSGKLTQKLVTAGVMAGITASTLLYADSLTAEAMTA

SEQ ID NO: 30 - protein, 41 aa, Artificial Sequence
  targeting sequence
MKSFITRNKTAILATVAATGTAIGAYYYYNQLQQQQQRGKK

SEQ ID NO: 31 - protein, 4 aa, Artificial Sequence
  targeting sequence
KDEL

SEQ ID NO: 32 - protein, 15 aa, Artificial Sequence
  targeting sequence
LYLSRRSFIDEKKMP

SEQ ID NO: 33 - protein, 19 aa, Artificial Sequence
  targeting sequence
LNPPDESGPGCMSCKCVLS

SEQ ID NO: 34 - protein, 15 aa, Artificial Sequence
  targeting sequence
LTEPTQPTRNQCCSN

SEQ ID NO: 35 - protein, 9 aa, Artificial Sequence
  targeting sequence
RTALGDIGN

SEQ ID NO: 36 - protein, 20 aa, Artificial Sequence
  signal sequence
MYRMQLLSCIALSLALVTNS

SEQ ID NO: 37 - protein, 29 aa, Artificial Sequence
  signal sequence
MATGSRTSLLLAFGLLCLPWLQEGSAFPT

SEQ ID NO: 38 - protein, 27 aa, Artificial Sequence
  signal sequence
MALWMRLLPLLALLALWGPDPAAAFVN

SEQ ID NO: 39 - protein, 18 aa, Artificial Sequence
  signal sequence
MKAKLLVLLYAFVAGDQI

SEQ ID NO: 40 - protein, 24 aa, Artificial Sequence
  signal leader sequence
MGLTSQLLPPLFFLLACAGNFVHG

SEQ ID NO: 41 - protein, 4 aa, Artificial Sequence
  signal sequence
KDEL

SEQ ID NO: 42 - protein, 4 aa, Artificial Sequence
  signal sequence
DDEL

SEQ ID NO: 43 - protein, 4 aa, Artificial Sequence
  signal sequence
DEEL

SEQ ID NO: 44 - protein, 4 aa, Artificial Sequence
  signal sequence
QEDL

SEQ ID NO: 45 - protein, 4 aa, Artificial Sequence
  signal sequence
RDEL

SEQ ID NO: 46 - protein, 7 aa, Artificial Sequence
  signal sequence
PKKKRKV

SEQ ID NO: 47 - protein, 7 aa, Artificial Sequence
  signal sequence
PQKKIKS

SEQ ID NO: 48 - protein, 5 aa, Artificial Sequence
  signal sequence
QPKKP

SEQ ID NO: 49 - protein, 4 aa, Artificial Sequence
  signal sequence
RKKR

SEQ ID NO: 50 - protein, 12 aa, Artificial Sequence
  signal sequence
RKKRRQRRRAHQ

SEQ ID NO: 51 - protein, 16 aa, Artificial Sequence
  signal sequence
RQARRNRRRRWRERQR

SEQ ID NO: 52 - protein, 19 aa, Artificial Sequence
  signal sequence
MPLTRRRPAASQALAPPTP

SEQ ID NO: 53 - protein, 15 aa, Artificial Sequence
  signal sequence
MDDQRDLISNNEQLP

SEQ ID NO: 54 - protein, 32 aa, Artificial Sequence
  signal sequence
MLFNLRXXLNNAAFRHGHNFMVRNFRCGQPLX

SEQ ID NO: 55 - protein, 8 aa, Artificial Sequence
  signal sequence
GCVCSSNP

SEQ ID NO: 56 - protein, 8 aa, Artificial Sequence
  signal sequence
GQTVTTPL

SEQ ID NO: 57 - protein, 8 aa, Artificial Sequence
  signal sequence
GQELSQHE

SEQ ID NO: 58 - protein, 8 aa, Artificial Sequence
  signal sequence
GNSPSYNP

SEQ ID NO: 59 - protein, 8 aa, Artificial Sequence
  signal sequence
GVSGSKGQ

SEQ ID NO: 60 - protein, 8 aa, Artificial Sequence
  signal sequence
GQTITTPL

SEQ ID NO: 61 - protein, 8 aa, Artificial Sequence
  signal sequence
GQTLTTPL

SEQ ID NO: 62 - protein, 8 aa, Artificial Sequence
  signal sequence
GQIFSRSA

SEQ ID NO: 63 - protein, 8 aa, Artificial Sequence
  signal sequence
GQIHGLSP

SEQ ID NO: 64 - protein, 8 aa, Artificial Sequence
  signal sequence
GARASVLS

SEQ ID NO: 65 - protein, 8 aa, Artificial Sequence
  signal sequence
GCTLSAEE

SEQ ID NO: 66 - protein, 8 aa, Artificial Sequence
  signal sequence
GQNLSTSN

SEQ ID NO: 67 - protein, 8 aa, Artificial Sequence
  signal sequence
GAALTILV

SEQ ID NO: 68 - protein, 8 aa, Artificial Sequence
  signal sequence
GAALTLLG

SEQ ID NO: 69 - protein, 8 aa, Artificial Sequence
  signal sequence
GAQVSSQK

SEQ ID NO: 70 - protein, 8 aa, Artificial Sequence
  signal sequence
GAQLSRNT

SEQ ID NO: 71 - protein, 8 aa, Artificial Sequence
  signal sequence
GNAAAAKK

SEQ ID NO: 72 - protein, 8 aa, Artificial Sequence
  signal sequence
GNEASYPL

SEQ ID NO: 73 - protein, 8 aa, Artificial Sequence
  signal sequence
GSSKSKPK

SEQ ID NO: 74 - DNA, 1555 bp, Artificial Sequence
  pVHE transfer plasmid
ggccaaaaattgaaaaactagatctatttattgcacgcggccgcaaaccatgggatggagctgtatcatcctcttcttggtagcaacagctacaggcgcgcatatggtcaccgtctcctcagggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgctgagctgcctcccaaagtgagcgtcttcgtcccaccccgcgacggcttcttcggcaacccccgcagcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgactagcacactgaccatcaaagagagcgactggctcagccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcactgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatgagtcgac

SEQ ID NO: 75 - DNA, 446 bp, Artificial Sequence
  pVKE transfer plasmid
ggccaaaaattgaaaaactagatctatttattgcacgcggccgcccatgggatggagctgtatcatcctcttcttggtagcaacagctacaggcgtgcacttgactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggtcgac

SEQ ID NO: 76 - DNA, 455 bp, Artificial Sequence
  pVLE transfer plasmid
ggccaaaaattgaaaaactagatctatttattgcacgcggccgcccatgggatggagctgtatcatcctcttcttggtagcaacagctacaggcgtgcacttgactcgagaagcttaccgtcctacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaggtcgac

SEQ ID NO: 77 - protein, 9 aa, Artificial Sequence
  epitope
GYKAGMIHI

SEQ ID NO: 78 - DNA, 47 bp, Artificial Sequence
  cassette with multiple restriction sites
gcggccgcaaaccatggaaagcgcgcatatggtcaccaaaagtcgac

SEQ ID NO: 79 - DNA, 23 bp, Artificial Sequence
  primer
attaggtcaccgtctcctcaggg

SEQ ID NO: 80 - DNA, 31 bp, Artificial Sequence
  primer
attagtcgactcatggaagaggcacgttctt

SEQ ID NO: 81 - DNA, 51 bp, Artificial Sequence
  cassette with multiple restriction sites
gcggccgcccatggatagcgtgcacttgactcgagaagcttagtagtcgac

SEQ ID NO: 82 - DNA, 30 bp, Artificial Sequence
  primer
cacgactcgagatcaaacgaactgtggctg

SEQ ID NO: 83 - DNA, 38 bp, Artificial Sequence
  primer
aatatgtcgacctaacactctcccctgttgaagctctt

SEQ ID NO: 84 - DNA, 40 bp, Artificial Sequence
  primer
atttaagcttaccgtcctacgaactgtggctgcaccatct

SEQ ID NO: 85 - DNA, 39 bp, Artificial Sequence
  oligonucleotide primer
aatatgcgcgcactcccaggtgcagctggtgcagtctgg

SEQ ID NO: 86 - DNA, 39 bp, Artificial Sequence
  Oligonucleotide primer
aatatgcgcgcactcccaggtcaccttgaaggagtctgg

SEQ ID NO: 87 - DNA, 39 bp, Artificial Sequence
  oligonucleotide primer
aatatgcgcgcactccgaggtgcagctggtggagtctgg

SEQ ID NO: 88 - DNA, 39 bp, Artificial Sequence
  oligonucleotide primer
aatatgcgcgcactcccaggtgcagctgcaggagtcggg

SEQ ID NO: 89 - DNA, 38 bp, Artificial Sequence
  oligonucleotide primer
aatatgcgcgcactccgaggtgcagctggtgcagtctg

SEQ ID NO: 90 - DNA, 29 bp, Artificial Sequence
  oligonucleotide primer
gagacggtgaccagggtgccctggcccca

SEQ ID NO: 91 - DNA, 29 bp, Artificial Sequence
  oligonucleotide primer
gagacggtgaccagggtgccacggcccca

SEQ ID NO: 92 - DNA, 29 bp, Artificial Sequence
  oligonucleotide primer
gagacggtgaccattgtcccttggcccca

SEQ ID NO: 93 - DNA, 29 bp, Artificial Sequence
  oligonucleotide primer
gagacggtgaccagggttccctggcccca

SEQ ID NO: 94 - DNA, 29 bp, Artificial Sequence
  oligonucleotide primer
gagacggtgaccgtggtcccttggcccca

SEQ ID NO: 95 - DNA, 37 bp, Artificial Sequence
  oligonucleotide primer
caggagtgcactccgacatccagatgacccagtctcc

SEQ ID NO: 96 - DNA, 37 bp, Artificial Sequence
  oligonucleotide primer
caggagtgcactccgatgttgtgatgactcagtctcc

SEQ ID NO: 97 - DNA, 37 bp, Artificial Sequence
  oligonucleotide primer
caggagtgcactccgaaattgtgttgacgcagtctcc

SEQ ID NO: 98 - DNA, 37 bp, Artificial Sequence
  oligonucleotide primer
caggagtgcactccgacatcgtgatgacccagtctcc

SEQ ID NO: 99 - DNA, 37 bp, Artificial Sequence
  oligonucleotide primer
caggagtgcactccgaaacgacactcacgcagtctcc

SEQ ID NO: 100 - DNA, 37 bp, Artificial Sequence
  oligonucleotide primer
caggagtgcactccgaaattgtgctgactcagtctcc

SEQ ID NO: 101 - DNA, 29 bp, Artificial Sequence
  oligonucleotide primer
ttgatctcgagcttggtcccttggccgaa

SEQ ID NO: 102 - DNA, 29 bp, Artificial Sequence
  oligonucleotide primer
ttgatctcgagcttggtcccctggccaaa

SEQ ID NO: 103 - DNA, 29 bp, Artificial Sequence
  oligonucleotide primer
ttgatctcgagtttggtcccagggccgaa

SEQ ID NO: 104 - DNA, 29 bp, Artificial Sequence
  oligonucleotide primer
ttgatctcgagcttggtccctccgccgaa

SEQ ID NO: 105 - DNA, 29 bp, Artificial Sequence
  oligonucleotide primer
ttaatctcgagtcgtgtcccttggccgaa

SEQ ID NO: 106 - DNA, 37 bp, Artificial Sequence
  oligonucleotide primer
cagatgtgcactcccagtctgtgttgacgcagccgcc

SEQ ID NO: 107 - DNA, 37 bp, Artificial Sequence
  oligonucleotide primer
cagatgtgcactcccagtctgccctgactcagcctgc

SEQ ID NO: 108 - DNA, 37 bp, Artificial Sequence
  oligonucleotide primer
cagatgtgcactcctcctatgtgctgactcagccacc

SEQ ID NO: 109 - DNA, 37 bp, Artificial Sequence
  oligonucleotide primer
cagatgtgcactcctcttctgagctgactcaggaccc

SEQ ID NO: 110 - DNA, 37 bp, Artificial Sequence
  oligonucleotide primer
cagatgtgcactcccacgttatactgactcaaccgcc

SEQ ID NO: 111 - DNA, 37 bp, Artificial Sequence
  oligonucleotide primer
cagatgtgcactcccaggctgtgctcactcagccgtc

SEQ ID NO: 112 - DNA, 37 bp, Artificial Sequence
  oligonucleotide primer
cagatgtgcactccaattttatgctgactcagcccca

SEQ ID NO: 113 - DNA, 37 bp, Artificial Sequence
  oligonucleotide primer
cagatgtgcactcccaggctgtggtgactcaggagcc

SEQ ID NO: 114 - DNA, 29 bp, Artificial Sequence
  oligonucleotide primer
acggtaagcttggtcccagttccgaagac

SEQ ID NO: 115 - DNA, 29 bp, Artificial Sequence
  oligonucleotide primer
acggtaagcttggtccctccgccgaatac

SEQ ID NO: 116 - protein, 19 aa, Artificial Sequence
  localization signal
MGWSCIILFLVATATGAHS

SEQ ID NO: 117 - protein, 26 aa, Artificial Sequence
  localization signal
NLWTTASTFIVLFLLSLFYSTTVTLF

SEQ ID NO: 118 - protein, 4 aa, Artificial Sequence
  localization signal
KDEL

SEQ ID NO: 119 - protein, 7 aa, Artificial Sequence
  localization signal
PKKKRKV

SEQ ID NO: 120 - protein, 14 aa, Artificial Sequence
  localization signal
MGSSKSKPKDPSQR

SEQ ID NO: 121 - protein, 19 aa, Artificial Sequence
  localization signal
LNPPDESGPGCMSCKCVLS

SEQ ID NO: 122 - protein, 5 aa, Artificial Sequence
  localization signal
KFERQ

SEQ ID NO: 123 - protein, 29 aa, Artificial Sequence
  localization signal
MSVLTPLLLRGLTGSARRLPVPRAKIHSL

SEQ ID NO: 124 - DNA, 24 bp, Artificial Sequence
  forward primer
gccaccatgggccctaaaaagaag

SEQ ID NO: 125 - DNA, 29 bp, Artificial Sequence
  reverse primer
attagcgcgctcccaccgtactcgtcaat

SEQ ID NO: 126 - DNA, 21 bp, Artificial Sequence
  primer
atgttacgtcctgtagaaacc

SEQ ID NO: 127 - DNA, 21 bp, Artificial Sequence
  primer
tcattgtttgcctccctgctg

SEQ ID NO: 128 - DNA, 28 bp, Artificial Sequence
  primer
aaagcggccgccccgggatgttacgtcc

SEQ ID NO: 129 - DNA, 29 bp, Artificial Sequence
  primer
aaagggcccggcgcgcctcattgtttgcc

SEQ ID NO: 130 - DNA, 37 bp, Artificial Sequence
  primer
aaaggatccataatgaattcagtgactgtatcacacg

SEQ ID NO: 131 - DNA, 34 bp, Artificial Sequence
  primer
cttgcggccgcttaataaataaacccttgagccc

SEQ ID NO: 132 - DNA, 34 bp, Artificial Sequence
  primer
attgagctcttaatacttttgtcgggtaacagag

SEQ ID NO: 133 - DNA, 29 bp, Artificial Sequence
  primer
ttactcgagagtgtcgcaatttggatttt

SEQ ID NO: 134 - DNA, 29 bp, Artificial Sequence
  primer
aaagaattcctttattgtcatcggccaaa

SEQ ID NO: 135 - DNA, 30 bp, Artificial Sequence
  primer
aatctgcagtcattgtttgcctccctgctg

SEQ ID NO: 136 - DNA, 37 bp, Artificial Sequence
  primer
aaagaattcataatgaattcagtgactgtatcacacg

SEQ ID NO: 137 - DNA, 32 bp, Artificial Sequence
  primer
cttggatccttaataaataaacccttgagccc

SEQ ID NO: 138 - DNA, 29 bp, Artificial Sequence
  primer
aataagctttgactccagatacatatgga

SEQ ID NO: 139 - DNA, 25 bp, Artificial Sequence
  primer
aatctgcagcaccagttccatcttt

SEQ ID NO: 140 - DNA, 23 bp, Artificial Sequence
  primer
aatggatcctcatccagcggcta

SEQ ID NO: 141 - DNA, 27 bp, Artificial Sequence
  primer
aatgagctctagtacctacaacccgaa

SEQ ID NO: 142 - DNA, 28 bp, Artificial Sequence
  primer
aaagtcgacggccaaaaattgaaatttt

SEQ ID NO: 143 - DNA, 25 bp, Artificial Sequence
  primer
aatggatcctcattgtttgcctccc

SEQ ID NO: 144 - DNA, 78 bp, Artificial Sequence
  nucleotide cassette
ctcgagatcaaagagggtaaatcttccggatctggttccgaaggcgcgcatgcggtcaccgtctcctcatgagtcgac

SEQ ID NO: 145 - DNA, 42 bp, Artificial Sequence
  linker
gagggtaaatcttccggatctggttccgaaggcgcgcactcc

SEQ ID NO: 146 - protein, 14 aa, Artificial Sequence
  linker
EGKSSGSGSEGAHS

SEQ ID NO: 147 - DNA, 81 bp, Artificial Sequence
  nucleotide cassette
aagcttaccgtcctagagggtaaatcttccggatctggttccgaaggcgcgcatgcggtcaccgtctcctcatgagtcgac

SEQ ID NO: 148 - DNA, 42 bp, Artificial Sequence; identical to SEQ ID NO: 145
  linker

SEQ ID NO: 149 - protein, 14 aa, Artificial Sequence
  linker
EGKSSGSGSEGAHS

SEQ ID NO: 150 - DNA, 57 bp, Artificial Sequence
  p7.5/tk vector
ggccaaaaattgaaaaactagatctatttattgcacgcggccgccatgggcccggcc

SEQ ID NO: 151 - DNA, 145 bp, Artificial Sequence
  p 7.5/ATG0/tk promoter
ggccaaaaattgaaaaactagatctatttattgcacgcggccgccgtggatcccccgggctgcaggaattcgatatcaagcttatcgataccgtcgacctcgagggggggcctaactaactaattttgtttttgtgggcccggcc

SEQ ID NO: 152 - DNA, 148 bp, Artificial Sequence
  p7.5/ATG1/tk
ggccaaaaattgaaaaactagatctatttattgcacgcggccgccatggtggatcccccgggctgcaggaattcgatatcaagcttatcgataccgtcgacctcgagggggggcctaactaactaattttgtttttgtgggcccggcc

SEQ ID NO: 153 - DNA, 149 bp, Artificial Sequence
  p7.5/ATG2/tk
ggccaaaaattgaaaaactagatctatttattgcacgcggccgccatgagtggatcccccgggctgcaggaattcgatatcaagcttatcgataccgtcgacctcgagggggggcctaactaactaattttgtttttgtgggcccggcc

SEQ ID NO: 154 - DNA, 11 bp, Artificial Sequence
  generic VH primer
gcgcgcactcc